(19) United States
(12) Patent Application Publication
(10) Pub. No.: US 2009/0197269 A1
(43) Pub. Date: Aug. 6, 2009

(54) Translocation and mutant CSF1R Kinase in human leukemia

(75) Inventors: Ting-Lei Gu, Woburn, MA (US); Valerie Goss, Seabrook, NH (US); Robert Polakiewicz, Lexington, MA (US); Brian Druker, Portland, OR (US); Denise Walters, Rochester, MN (US)

Correspondence Address: Nancy Chiu Wilker, Ph.D.;Chief Intellectual Property Counsel, CELL SIGNALING TECHNOLOGY, INC., 3 Trask Lane, Danvers, MA, 01923, US

(73) Assignee: CELL SIGNALING TECHNOLOGY, INC.

(21) Appl. No.: 12/214,616

(22) Filed: Jun. 20, 2008

(30) Foreign Application Priority Data
Dec. 21, 2006 (US) PCT/US2006/048867

(51) Int. Cl. C12Q 1/68; C12N 15/11; C12N 15/00; C12N 15/87; C12N 5/06; C12P 21/00; C12N 9/96; C07K 16/18; G01N 33/574; C12Q 1/48

(52) U.S. Cl. 435/6; 536/23.2; 435/320.1; 435/455; 435/325; 435/69.7; 435/188; 530/391.3; 435/7.23; 435/15; 435/375

(57) ABSTRACT

In accordance with the invention, a novel gene translocation, (3p21, 5q33), in human myelogenous leukemia (AML) that results in a fusion protein combining part of RNA Binding Protein-6 (RBM6) with Macrophage Colony Stimulating Factor-1 Receptor (CSF1R) kinase has now been identified. The RBM6-CSF1R fusion protein and truncated CSF1R kinase itself, which both retain CSF1R tyrosine kinase activity, were confirmed to drive the proliferation and survival of acute megakaryoblastic leukemia (AML-M7). The invention therefore provides, in part, isolated polynucleotides and vectors encoding the disclosed mutant CSF1R kinase polypeptides, probes for detecting it, isolated mutant polypeptides, recombinant polypeptides, and reagents for detecting the fusion and truncated polypeptides. The disclosed identification of this new fusion protein and truncated kinase enables new methods for determining the presence of these mutant CSF1R kinase polypeptides in a biological sample, methods for screening for compounds that inhibit the proteins, and methods for inhibiting the progression of a cancer characterized by the mutant polynucleotides or polypeptides, which are also provided by the invention.

### RELATED APPLICATIONS

This application claims priority to and is a Chapter II National Stage Entry in the U.S.P.T.O. of PCT/US2006/048867 filed Dec. 21, 2006 which itself claims priority to and the benefit of, U.S. Ser. No. 60/752,474, filed Dec. 21, 2005, presently abandoned, both disclosures of which are hereby incorporated herein in their entirety.

### JOINT RESEARCH AGREEMENT

This application describes and claims certain subject matter that was developed under a written joint collaborative research agreement between CELL SIGNALING TECHNOLOGY, INC., and THE OREGON HEALTH & SCIENCE UNIVERSITY, having an effective date of May 4, 2004, pertaining to markers of cancer and drug resistance.

### FIELD OF THE INVENTION

The invention relates generally to proteins and genes involved in cancer, and to the detection, diagnosis and treatment of cancer.

### BACKGROUND OF THE INVENTION

Many cancers are characterized by disruptions in cellular signaling pathways that lead to aberrant control of cellular processes, or to uncontrolled growth and proliferation of cells. These disruptions are often caused by changes in the activity of particular signaling proteins, such as kinases. Among these cancers are leukemias, which include four major subtypes, acute lymphocytic leukemia, chronic lymphocytic leukemia, acute myelogenous leukemia (AML), and chronic myelogenous leukemia. There are about 35,000 new cases of AML in the United States annually, and it is estimated that almost 23,000 patients will die each year from the disease in the United States alone. See “Cancer Facts and Figures 2005,” American Cancer Society. Among the four subtypes of leukemia, AML, which itself includes various subtypes such as acute megakaryoblastic leukemia (AML-M7), is the most common and most deadly.

It is known that gene translocations resulting in kinase fusion proteins with aberrant signaling activity can directly lead to certain cancers. For example, it has been directly demonstrated that the BCR-ABL oncoprotein, a tyrosine kinase fusion protein, is the causative agent in human chronic myelogenous leukemia (CML). The BCR-ABL oncoprotein, which is found in at least 90-95% of CML cases, is generated by the translocation of gene sequences from the c-ABL protein tyrosine kinase on chromosome 9 into BCR sequences on chromosome 22, producing the so-called Philadelphia chromosome. See, e.g. Kurzock et al., N. Engl. J. Med. 319: 990-998 (1988). The translocation is also observed in acute lymphocytic leukemia and AML cases.

A limited number of gene translocations leading to mutant or fusion proteins implicated in a variety of other hematological cancers have been described. See, e.g., review by Falini et al., Blood 99(2): 409-426 (2002). Among such translocations are the NPM-ALK fusion (involved in anaplastic large cell lymphoma), the E2A-PBX/HLF fusion (involved in B-cell acute lymphoblastic leukemia), and the NPM-MLF1 fusion (involved in myelodysplastic syndrome/AML). See id. In AML, the translocation of OTT-MAL at t(1;22)(p13;q13) has been described (see Mercher et al., Genes Chromosomes Cancer 33(1): 22-8 (2002)), as has the AML1-MTG8 t(8:21) translocation (see Ohki et al., U.S. Pat. No. 5,580,727 (December 1996)). Defects in RBM-6 expression and/or activity have been found in small cell and non-small cell lung carcinomas. See Drabkin et al., Oncogene 8(16): 2589-97 (1999).

Defects in CSF1R expression and/or activation have been found in acute myeloid leukemia and myelodysplastic syndrome (MDS). See, e.g. Casas et al., Leuk. Lymphoma 44:1935-1941 (2003); Li et al., Leukemia Res. 26: 377-382 (2002). Elevated coexpression CSF1R and its ligand, CSF1, have been correlated with invasiveness and poor prognosis of epithelial tumors including breast, ovarian and endometrial cancer. See Kacinski B M, Ann. Med. 27: 79-85 (1995). Activating point mutations at codons L301 and Y969 of CSF1R have been detected in AML and CMML (see Ridge et al., Proc Natl Acad Sci USA 87(4): 1377-80 (1990); Tobal et al., Leukemia 4(7): 486-89 (1990)).

Identifying translocations and mutations in human cancers is highly desirable because it can lead to the development of new therapeutics that target such mutant or fusion proteins, and to new diagnostics for identifying patients that have such gene translocations. For example, BCR-ABL has become a target for the development of therapeutics to treat leukemia. Recently, Gleevec® (Imatinib mesylate, STI-571), a small molecule inhibitor of the ABL kinase, has been approved for the treatment of CML. This drug is the first of a new class of anti-proliferative agents designed to interfere with the signaling pathways that drive the growth of tumor cells. The development of this drug represents a significant advance over the conventional therapies for CML and ALL, chemotherapy and radiation, which are plagued by well known side-effects and are often of limited effect since they fail to specifically target the underlying causes of the malignancies. Likewise, reagents and methods for specifically detecting BCR-ABL fusion protein in patients, in order to identify patients most likely to respond to targeted inhibitors like Gleevec®, have been described.

Accordingly, there remains a need for the identification of novel gene translocations or mutations resulting in fusion or mutant proteins implicated in the progression of human cancers, including leukemias, and the development of new reagents and methods for the study and detection of such mutant/fusion proteins. Identification of such proteins will, among other things, desirably enable new methods for selecting patients for targeted therapies, as well as for the screening of new drugs that inhibit such mutant/fusion proteins.

### SUMMARY OF THE INVENTION

In accordance with the invention, a novel gene translocation, (3p21, 5q33), in human myelogenous leukemia (AML) that results in a fusion protein combining part of RNA Binding Protein-6 (RBM6) with Macrophage Colony Stimulating Factor-1 Receptor (CSF1R) kinase has now been identified. The mutant CSF1R kinase, which retains tyrosine kinase activity independent of the RBM6 fusion, was confirmed to drive the proliferation and survival of a human acute megakaryoblastic leukemia (AML-M7) cell line, MKPL-1.

The invention therefore provides, in part, isolated polynucleotides and vectors encoding the disclosed mutant CSF1R polypeptides, probes and assays for detecting them, isolated mutant CSF1R polypeptides, recombinant mutant polypeptides, and reagents for detecting the mutant CSF1R polynucleotides and polypeptides. The disclosed identification of this new mutant CSF1R kinase protein and RBM6 translocation enables new methods for determining the presence of mutant CSF1R polynucleotides or polypeptides in a biological sample, methods for screening for compounds that inhibit the mutant kinase protein, and methods for inhibiting the progression of a cancer characterized by the expression of mutant CSF1R polynucleotides or polypeptides, which are also provided by the invention. The aspects and embodiments of the invention are described in more detail below.

### BRIEF DESCRIPTION OF THE DRAWINGS

FIG. 1—shows the location of the RBM-6 gene and CSF1R gene on chromosomes 3 and 5 respectively (panel A), and the domain locations of full length RBM-6 and CSF1R proteins as well as those of RBM6-CSF1R fusion protein (panel B); the fusion junction occurs at residue 574 in the juxtamembrane domain of CSF1R.

FIG. 2—is the amino acid sequence (1 letter code) of human RBM6-CSF1R fusion protein (SEQ ID NO: 1) (top panel) with coding DNA sequence also indicated (SEQ ID NO: 2) (bottom panel); the residues of the RBM-6 moiety are in italics, while the residues of the split kinase domain of CSF1R are in bold.

FIG. 3A-3B—is the amino acid sequence (1 letter code) of human RBM-6 protein (SEQ ID NO: 3) (SwissProt Accession No. P78332) with coding DNA sequence also indicated (SEQ ID NO: 4) (GeneBank Accession No. NM_{—}005777); the residues involved in the translocation are underlined.

FIG. 4A-4B—is the amino acid sequence (1 letter code) of human CSF1R kinase (SEQ ID NO: 5) (SwissProt Accession No. P07333) with coding DNA sequence also indicated (SEQ ID NO: 6) (GeneBank Accession No. NM_{—}005211); the residues involved in the translocation are underlined, while the residues of the split kinase domain are in bold.

FIG. 5—is a Western blot analysis of extracts from a human AML cell line (MKPL-1) showing expression of a truncated/fusion form of CSF1R.

FIG. 6—is a Western blot analysis of extracts from a human AML cell line (MKPL-1) showing inhibition of CSF1R kinase activity, as well as its downstream target STAT5 and ERK by Gleevec® (panel A), and a graph depicting the inhibition of cell growth in various cell types by Imatinib (Gleevec®) in a 48 hour MTT assay demonstrating that growth of MKPL-1 is specifically inhibited by Imatinib (Gleevec®) (panel B). It is accompanied by an increase in apoptosis (panel C).

FIG. 7—is a Western blot analysis of extracts from a human AML cell line (MKPL-1) showing inhibition of CSF1R kinase activity, as well as its downstream target STAT5 and ERK by siRNA silencing (panel A), and a graph depicting the inhibition of cell growth in various cell types by siRNA silencing in a 72 hour MTT assay demonstrating that growth of MKPL-1 is specifically inhibited by siRNA against CSF1R protein (panel B). It is accompanied by an increase in apoptosis (panel C).

FIG. 8—is a gel depicting detection of CSF1R via the 5′ RACE product with CSF1R primers after 2 rounds of PCR; UAP stands for Universal Amplification Primer, GSP for Gene Specific Primer.

FIG. 9—are gels depicting the detection of the fusion gene formed by the RBM-6 and CSF1R translocation by RT-PCR; the DNA (and protein) sequence of the exon 2/exon 12 fusion junction is shown below (SEQ ID NO: 7 and SEQ ID NO: 8).

FIG. 10—presents (top) diagrams showing the location of exon 2 in the RBM-6 gene and exon 12 in the CSF1R gene that are involved in the translocation resulting in the fusion protein; also shown (bottom) are primer locations used for PCR amplification of the fusion protein, and a gel depicting the expected size of PCR product.

FIG. 11—are gels showing the cloning and expression of the RBM6-CSF1R fusion protein in Baf3 cells (panels A and B), and a graph depicting the IL3-independent growth of Baf3 cells expressing the RBM6-CSF1R fusion protein, as compared to parental BaF3 cells or BaF3 cells transfected with an empty vector (panel C).

### DETAILED DESCRIPTION OF THE INVENTION

In accordance with the invention, a previously unknown gene translocation that results in a mutant kinase fusion protein, RBM6-CSF1R, has now been identified in human acute megakaryoblastic leukemia (AML-M7), a subtype of acute myelogenous leukemia (AML). The translocation, which occurs between chromosome (3p21) and chromosome (5q33), produces a fusion protein that combines the N-terminus of RNA Binding Protein 6 (RBM-6), an 1123 amino acid RNA binding protein (also known as DEF-3), with the juxtamembrane and split kinase domains of Macrophage Colony Stimulating Factor-I Receptor (CSF1R), a 972 amino acid receptor tyrosine kinase. The resulting RBM6-CSF1R fusion protein, which is 435 amino acids and retains CSF1R kinase activity independent of the RBM6 moiety, was confirmed to drive the proliferation and survival of a human leukemia cell line, MKPL-1.

Although a few gene translocations that result in aberrant fusion proteins have been described in AML, including the translocation of OTT-MAL at t(1;22)(p13;q13) (see Mercher et al., supra.) and the AML1-MTG8 t(8:21) translocation (see Ohki et al., supra.), the presently disclosed RBM6/CSF1R translocation, fusion protein, and truncated active CSF1R mutant are novel. RBM-6 is an RNA binding protein that is expressed in most human tissues, and which specifically binds poly(G) RNA homopolymers. Defects in RBM-6 expression and/or activity have been found in small cell and non-small cell lung carcinomas. See Drabkin et al., supra. CSF1R, the product of the oncogene c-fms, is a transmembrane receptor tyrosine kinase that is the cell surface receptor for macrophage Colony Stimulating Factor-1 (CSF-1). CSF1R is expressed, in humans, in bone marrow and differentiated blood mononuclear cells, and it plays an important role in regulating the normal proliferation and differentiation of macrophages and trophoblasts. Defects in CSF1R expression and/or activation have been found in acute myeloid leukemia and myelodysplastic syndrome (MDS). See Casas et al., supra.; Li et al., supra. In addition, elevated coexpression CSF1R and its ligand, CSF1, has been correlated with invasiveness and poor prognosis of epithelial tumors including breast, ovarian and endometrial cancer. See Kacinski supra. Activating point mutations at codons L301 and Y969 of CSF1R have been detected in AML and CMML. See Ridge et al., supra.; Tobal et al., supra.

As further described below, the RBM6-CSF1R translocation and the expressed fusion protein have presently been isolated and sequenced, and cDNAs for expressing the mutant kinase protein (both as a fusion and as a truncated active kinase) produced. Accordingly, the invention provides, in part, isolated polynucleotides that encode RBM6-CSF1R fusion polypeptides or truncated active CSF1R polypeptides, nucleic acid probes that hybridize to such polynucleotides, and methods, vectors, and host cells for utilizing such polynucleotides to produce recombinant mutant CSF1R polypeptides. The invention also provides, in part, isolated polypeptides comprising amino acid sequences encoding RBM6-CSF1R fusion polypeptides or truncated active CSF1R polypeptides, recombinant mutant polypeptides, and isolated reagents that specifically bind to and/or detect RBM6-CSF1R fusion polypeptides, or truncated active CSF1R polypeptides, but do not bind to or detect either wild type RBM-6 or wild type CSF1R. These aspects of the invention, which are described in further detail below, will be useful, inter alia, in further studying the mechanisms of cancers driven by mutant CSF1R kinase expression/activity, for identifying leukemias and other cancers characterized by the RBM6-CSF1R translocation and/or fusion protein, or expression/activity of truncated active CSF1R kinase, and in practicing methods of the invention as further described below.

The identification of the novel CSF1R kinase mutant and translocation has important implications for the potential diagnosis and treatment of diseases, such as leukemia, that are characterized by this fusion protein. Leukemias, for example, are often difficult to diagnose until after they have advanced, increasing the difficulty of effectively treating or curing this disease. AML itself, the most common type of leukemia, is an aggressive disease, with a 5% year survival rate of less than 20%. See American Cancer Society, supra. The acute megakaryoblastic (AML-7) subtype of AML is a rare form of pediatric AML that is most prevalent in young children with Down's Syndrome. See A. Verschurr, Ophanet Encyclopedia (May 2004) (orpha.net/data/patho/GB/uk-AMLM7.pdf). Treatment of AML remains difficult, and the first-line therapy of aggressive multi-drug chemotherapy regimes is hard on patients and associated with significant mortality. Alternatively, total body irradiation coupled with chemotherapy and bone marrow transplant may be employed, which are similarly traumatic for patients.

Therefore, the discovery of the RBM6-CSF1R fusion protein resulting from gene translocation, which is presently shown to drive proliferation and survival of a subtype of AML (with truncated kinase activity independent of the RBM moiety), enables important new methods for accurately identifying mammalian leukemias (such as AML), as well as other cancers, in which the RBM6-CSF1R fusion protein or truncated active CSF1R kinase is expressed. These tumors are most likely to respond to inhibitors of the kinase activity of the CSF1R mutant protein, such as Imatinib (STI-571; Gleevec®). The ability to identify, as early as possible, cancers that are driven by a mutant CSF1R kinase will greatly assist in clinically determining which therapeutic, or combination of therapeutics, will be most appropriate for a particular patient, thus helping to avoid prescription of inhibitors targeting other kinases that are not, in fact, the primary signaling molecule driving the cancer.

Accordingly, the invention provides, in part, methods for detecting the presence of a RBM6-CSF1R translocation (t(3; 5)(p21, q33)) and/or fusion polypeptide, or a truncated CSF1R polynucleotide or truncated active CSF1R polypeptide, in a cancer using fusion-specific and mutant-specific reagents of the invention. Such methods may be practiced, for example, to identify a cancer, such as leukemia, that is likely to respond to an inhibitor of the CSF1R kinase activity of the mutant protein. The invention also provides, in part, methods for determining whether a compound inhibits the progression of a cancer characterized by a RBM6-CSF1R fusion polypeptide or truncated active CSF1R polypeptide. Further provided by the invention is a method for inhibiting the progression of a cancer that expresses a RBM6-CSF1R fusion polypeptide or truncated active CSF1R polypeptide by inhibiting the expression and/or activity of the mutant polypeptide. Such methods are described in further detail below.

The further aspects, advantages, and embodiments of the invention are described in more detail below. All references cited herein are hereby incorporated by reference in their entirety.

### DEFINITIONS

As used herein, the following terms have the meanings indicated.

“Antibody” or “antibodies” refers to all types of immunoglobulins, including IgG, IgM, IgA, IgD, and IgE, including F_{ab} or antigen-recognition fragments thereof, including chimeric, polyclonal, and monoclonal antibodies. The term “humanized antibody”, as used herein, refers to antibody molecules in which amino acids have been replaced in the non-antigen binding regions in order to more closely resemble a human antibody, while still retaining the original binding ability.

The term “biologically active” refers to a protein having structural, regulatory, or biochemical functions of a naturally occurring molecule. Likewise, “immunologically active” refers to the capability of the natural, recombinant, or synthetic RBM6-CSF1R fusion polypeptide or truncated active CSF1R polypeptide, or any oligopeptide thereof, to induce a specific immune response in appropriate animals or cells and to bind with specific antibodies.

The term “biological sample” is used in its broadest sense, and means any biological sample suspected of containing RBM6-CSF1R fusion or truncated CSF1R polynucleotides or polypeptides or fragments thereof, and may comprise a cell, chromosomes isolated from a cell (e.g., a spread of metaphase chromosomes), genomic DNA (in solution or bound to a solid support such as for Southern analysis), RNA (in solution or bound to a solid support such as for northern analysis), cDNA (in solution or bound to a solid support), an extract from cells, blood, urine, marrow, or a tissue, and the like.

“Characterized by” with respect to a cancer and mutant CSF1R polynucleotide and polypeptide is meant a cancer in which the RBM6-CSF1R gene translocation and/or expressed fusion polypeptide are present, or in which a truncated CSF1R polynucleotide and/or truncated active polypeptide are present, as compared to a cancer in which such translocation and/or fusion polypeptide are not present. The presence of mutant polypeptide may drive, in whole or in part, the growth and survival of such cancer.

“Consensus” refers to a nucleic acid sequence which has been re-sequenced to resolve uncalled bases, or which has been extended using XL-PCR™ (Perkin Elmer, Norwalk, Conn.) in the 5′ and/or the 3′ direction and re-sequenced, or which has been assembled from the overlapping sequences of more than one Incyte clone using the GELVIEW™ Fragment Assembly system (GCG, Madison, Wis.), or which has been both extended and assembled.

“CSF1R kinase-inhibiting therapeutic” means any composition comprising one or more compounds, chemical or biological, which inhibits, either directly or indirectly, the expression and/or activity of wild type or truncated active CSF1R kinase, either alone and/or as part of the RBM6-CSF1R fusion protein.

“Derivative” refers to the chemical modification of a nucleic acid sequence encoding RBM6-CSF1R fusion polypeptide or truncated active CSF1R polypeptide, or the encoded polypeptide itself. Illustrative of such modifications would be replacement of hydrogen by an alkyl, acyl, or amino group. A nucleic acid derivative would encode a polypeptide that retains essential biological characteristics of the natural molecule.

“Detectable label” with respect to a polypeptide, polynucleotide, or reagent disclosed herein means a chemical, biological, or other modification, including but not limited to fluorescence, mass, residue, dye, radioisotope, label, or tag modifications, etc., by which the presence of the molecule of interest may be detected.

“Expression” or “expressed” with respect to RBM6-CSF1R fusion polypeptide or truncated active CSF1R polypeptide in a biological sample means significantly expressed as compared to control sample in which this fusion polypeptide is not significantly expressed.

“Heavy-isotope labeled peptide” (used interchangeably with AQUA peptide) means a peptide comprising at least one heavy-isotope label, which is suitable for absolute quantification or detection of a protein as described in WO/03016861, “Absolute Quantification of Proteins and Modified Forms Thereof by Multistage Mass Spectrometry” (Gygi et al.), further discussed below. The term “specifically detects” with respect to such an AQUA peptide means the peptide will only detect and quantify polypeptides and proteins that contain the AQUA peptide sequence and will not substantially detect polypeptides and proteins that do not contain the AQUA peptide sequence.

“Isolated” (or “substantially purified”) refers to nucleic or amino acid sequences that are removed from their natural environment, isolated or separated. They preferably are at least 60% free, more preferably 75% free, and most preferably 90% or more free from other components with which they are naturally associated.

“Mimetic” refers to a molecule, the structure of which is developed from knowledge of the structure of RBM6-CSF1R fusion polypeptide, or truncated active CSF1R polypeptide, or portions thereof and, as such, is able to effect some or all of the actions of translocation associated protein-like molecules.

“Mutant CSF1R” polynucleotide or polypeptide means a RBM6-CSF1R fusion polynucleotide or polypeptide, or a truncated CSF1R polynucleotide or truncated active CSF1R polypeptide, as described herein.

“Polynucleotide” (or “nucleotide sequence”) refers to an oligonucleotide, nucleotide, or polynucleotide, and fragments or portions thereof, and to DNA or RNA of genomic or synthetic origin, which may be single- or double-stranded, and represent the sense or anti-sense strand.

“Polypeptide” (or “amino acid sequence”) refers to an oligopeptide, peptide, polypeptide, or protein sequence, and fragments or portions thereof, and to naturally occurring or synthetic molecules. Where “amino acid sequence” is recited herein to refer to an amino acid sequence of a naturally occurring protein molecule, “amino acid sequence” and like terms, such as “polypeptide” or “protein”, are not meant to limit the amino acid sequence to the complete, native amino acid sequence associated with the recited protein molecule.

“RBM6-CSF1R fusion polynucleotide” refers to the nucleic acid sequence of a substantially purified RBM6-CSF1R translocation gene product or fusion polynucleotide as described herein, obtained from any species, particularly mammalian, including bovine, ovine, porcine, murine, equine, and preferably human, from any source whether natural, synthetic, semi-synthetic, or recombinant.

“RBM6-CSF1R fusion polypeptide” refers to the amino acid sequence of a substantially purified RBM6-CSF1R fusion polypeptide described herein, obtained from any species, particularly mammalian, including bovine, ovine, porcine, murine, equine, and preferably human, from any source whether natural, synthetic, semi-synthetic, or recombinant.

The terms “specifically binds to” (or “specifically binding” or “specific binding”) in reference to the interaction of an antibody and a protein or peptide, mean that the interaction is dependent upon the presence of a particular structure (i.e. the antigenic determinant or epitope) on the protein; in other words, the antibody is recognizing and binding to a specific protein structure rather than to proteins in general. The term “does not bind” with respect to an antibody's binding to sequences or antigenic determinants other than that for which it is specific means does not substantially react with as compared to the antibody's binding to antigenic determinant or sequence for which the antibody is specific.

The term “stringent conditions” with respect to sequence or probe hybridization conditions is the “stringency” that occurs within a range from about Tₘ minus 5° C. (5° C. below the melting temperature (Tₘ) of the probe or sequence) to about 20° C. to 25° C. below Tₘ. Typical stringent conditions are: overnight incubation at 42° C. in a solution comprising: 50% formamide, 5×SSC (750 mM NaCl, 75 mM trisodium citrate), 50 mM sodium phosphate (pH 7.6), 5×Denhardt's solution, 10% dextran sulfate, and 20 micrograms/ml denatured, sheared salmon sperm DNA, followed by washing the filters in 0.1×SSC at about 65° C. As will be understood by those of skill in the art, the stringency of hybridization may be altered in order to identify or detect identical or related polynucleotide sequences.

“Truncated CSF1R [kinase] polynucleotide” refers to the nucleic acid sequence of a substantially purified truncated CSF1R polynucleotide that encodes an active truncated active CSF1R kinase polypeptide as described herein, obtained from any species, particularly mammalian, including bovine, ovine, porcine, murine, equine, and preferably human, from any source whether natural, synthetic, semi-synthetic, or recombinant.

“Truncated active CSF1R [kinase] polypeptide” refers to the amino acid sequence of a substantially purified, truncated CSF1R kinase polypeptide that retains kinase activity (and comprises the split kinase domain) but does not comprise the extracellular or transmembrane domains of wild type CSF1R kinase, as described herein, obtained from any species, particularly mammalian, including bovine, ovine, porcine, murine, equine, and preferably human, from any source whether natural, synthetic, semi-synthetic, or recombinant.

A “variant” of a mutant CSF1R polypeptide refers to an amino acid sequence that is altered by one or more amino acids. The variant may have “conservative” changes, wherein a substituted amino acid has similar structural or chemical properties, e.g., replacement of leucine with isoleucine. More rarely, a variant may have “nonconservative” changes, e.g., replacement of a glycine with a tryptophan. Similar minor variations may also include amino acid deletions or insertions, or both. Guidance in determining which amino acid residues may be substituted, inserted, or deleted without abolishing biological or immunological activity may be found using computer programs well known in the art, for example, DNASTAR software.

### A. Identification of Mutant CSF1R Kinase in Human Leukemia.

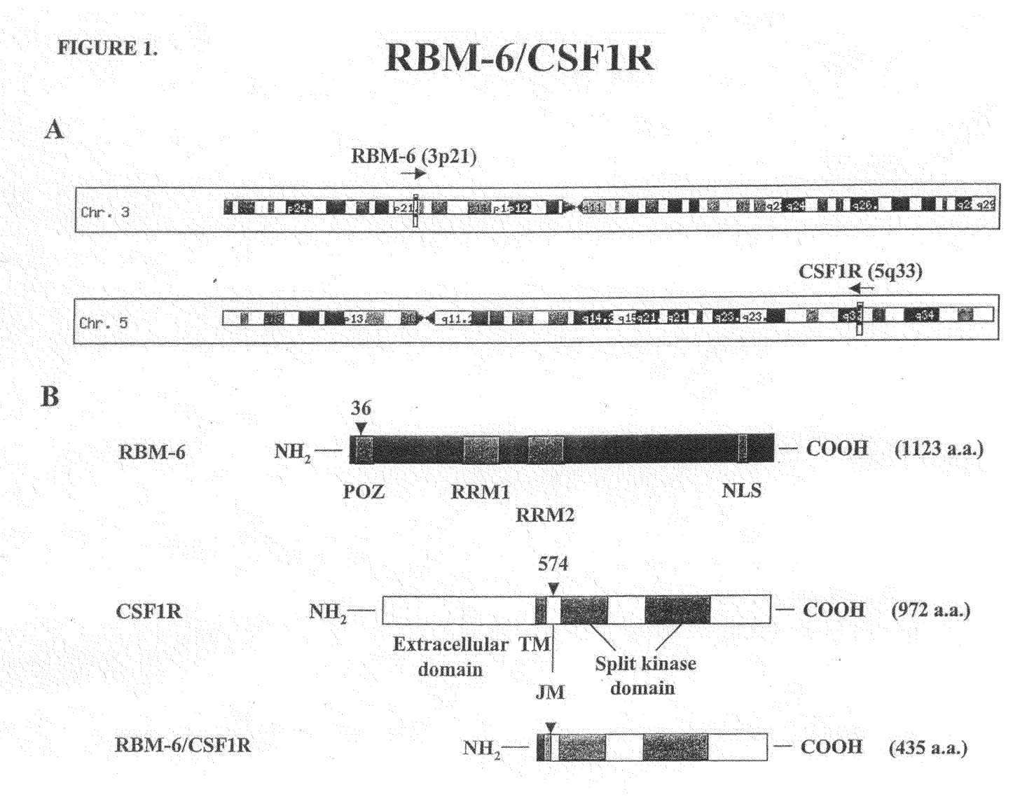

The novel human gene translocation disclosed herein, which occurs between chromosome (3p21) and chromosome (5q33) in human leukemia and results in expression of a fusion protein that combines the N-terminus of RBM-6 with the juxtamembrane and split kinase domains of CSF1R, was surprisingly identified during examination of global phosphorylated peptide profiles in extracts from a cell line (MKPL-1) of human acute megakaryoblastic leukemia (AML-M7), a subtype of acute myelogenous leukemia (AML). The chromosomes and genes involved in this translocation are shown in FIG. 1, panel A.

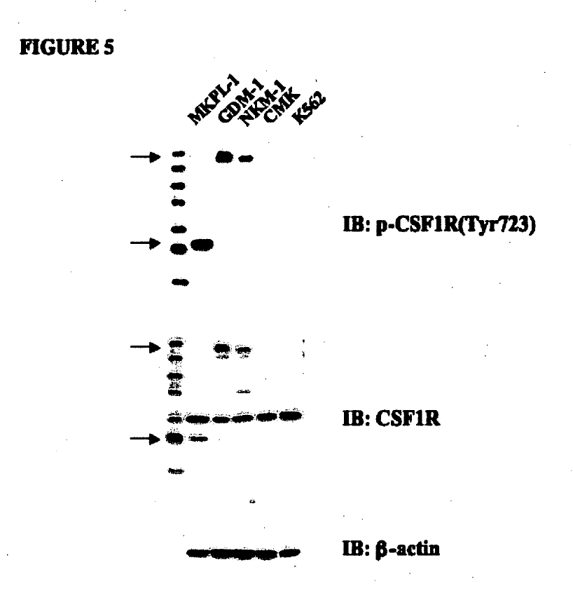

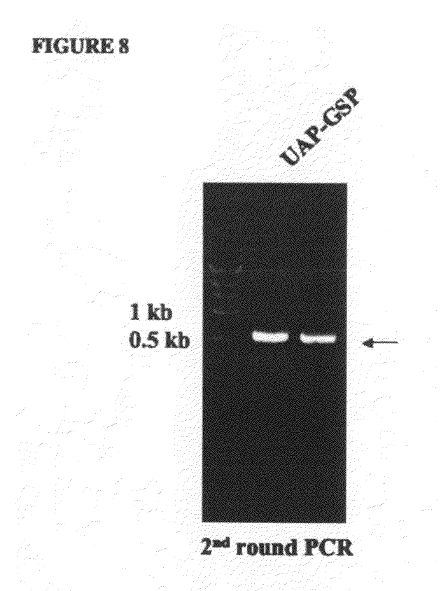

The phosphorylation profile of this cell line was elucidated using a recently described technique for the isolation and mass spectrometric characterization of modified peptides from complex mixtures (see U.S. Patent Publication No. 20030044848, Rush et al., “Immunoaffinity Isolation of Modified Peptides from Complex Mixtures” (the “IAP” technique), as further described in Example 1 below. Application of the IAP technique using a phosphotyrosine-specific antibody (CELL SIGNALING TECHNOLOGY, INC., Beverly, Mass., 2003/04 Cat. #9411), identified that the MKPL-1 cell line expresses CSF1R kinase, but that the kinase was apparently truncated (see FIG. 5). The screen identified many other activated kinases in the cell line including CSF1R. Analysis of the sequence 5′ to CSF1R by 5′ RACE then identified that the kinase was fused to the N-terminus of RBM-6 (see FIG. 8).

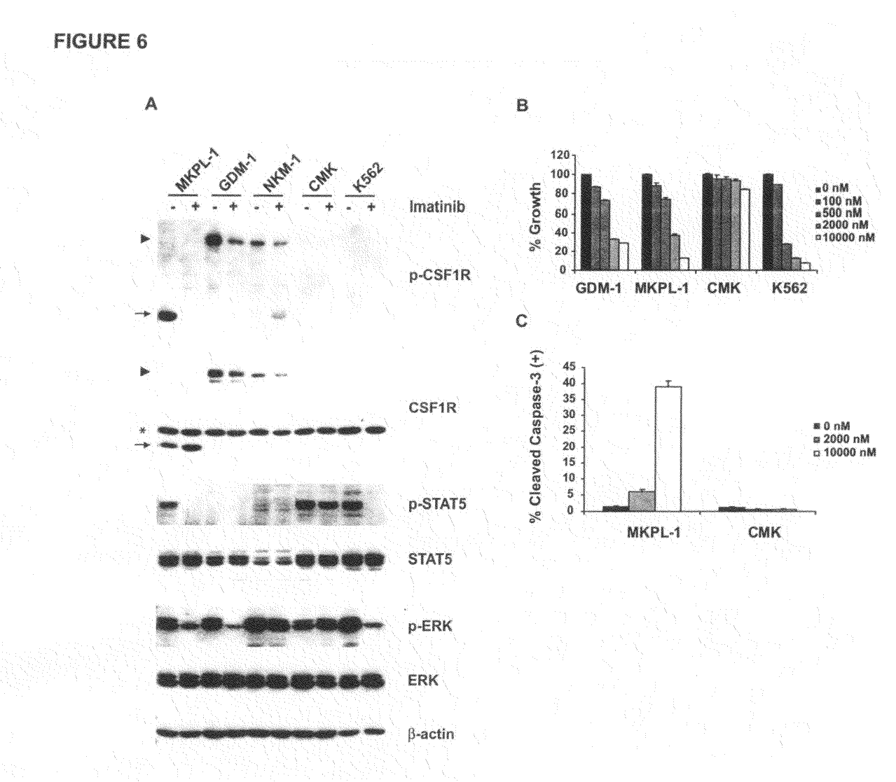

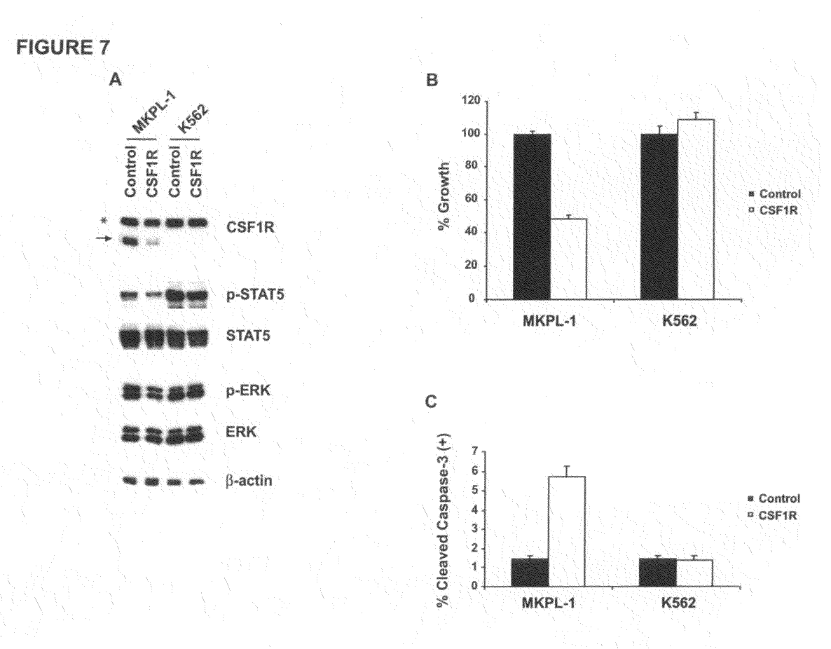

Expression of RBM6-CSF1R fusion polypeptide in the MKPL-1 cell line was then confirmed by Western blot analysis to examine both CSF1R kinase expression and phosphorylation of its downstream substrates (see Example 2; FIG. 6). Confirmation that the mutant CSF1R protein is driving cell proliferation and survival in this AML cell line was established by inhibiting the cells using both siRNA silencing and the known CSF1R targeted inhibitor, Gleevec® (Imatinib) (see Example 3; FIGS. 6 and 7).

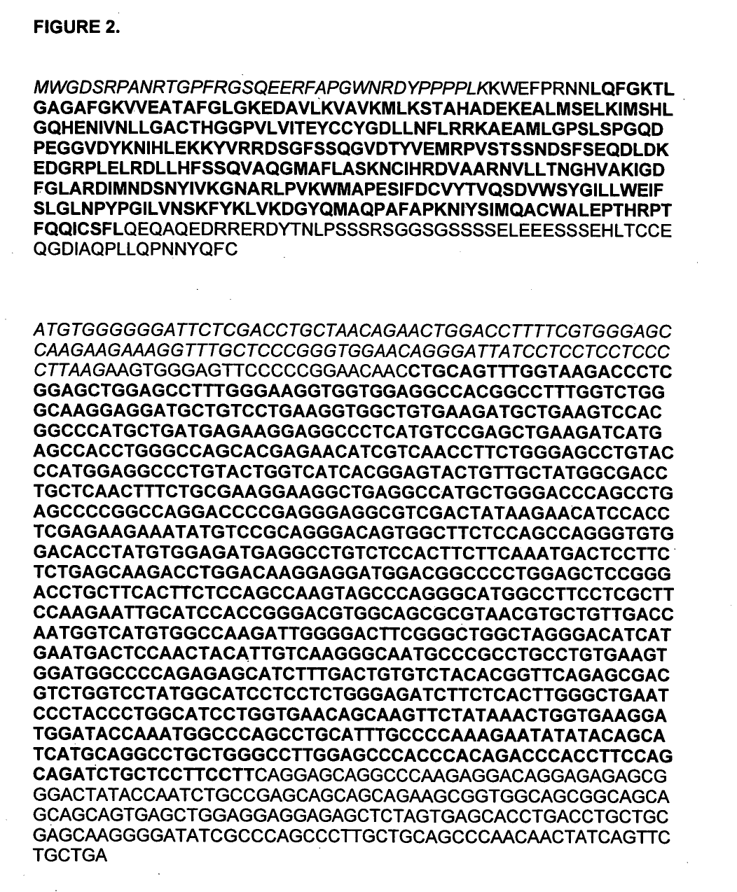

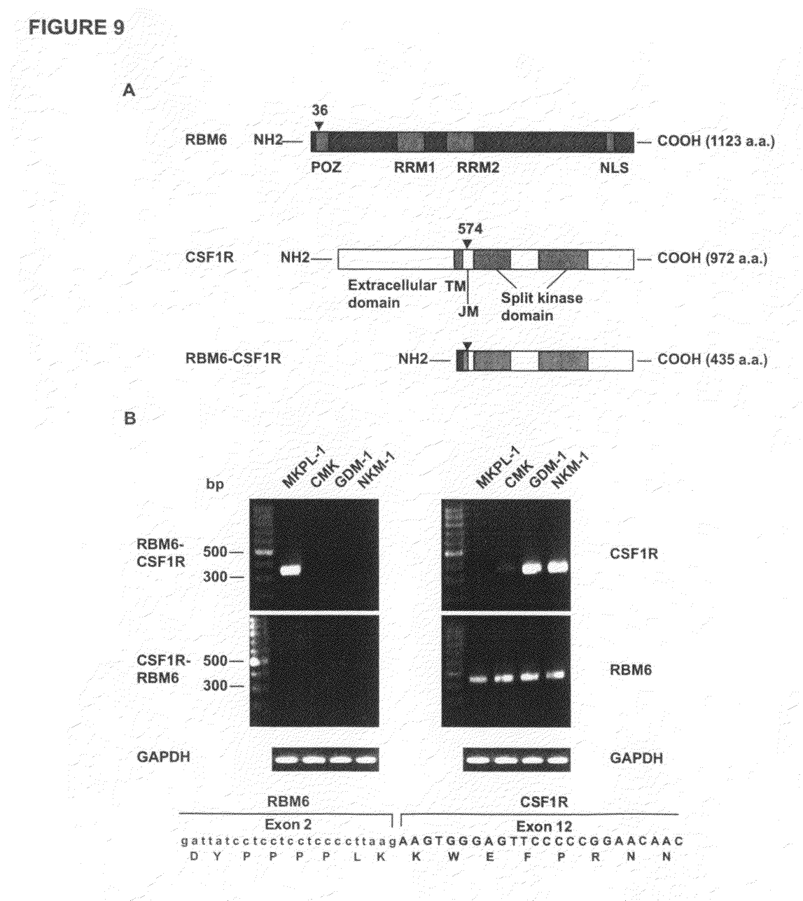

The RBM6-CSF1R fusion gene was amplified by PCR, isolated, and sequenced (see Example 4). As shown in panel B of FIG. 1, the RBM6-CSF1R translocation combines the N-terminus of RBM-6 (amino acids 1-36) with the juxtamembrane and split kinase domains of CSF1R (amino acids 574-972) (see also SEQ ID NOs: 3 and 5). The RBM6-CSF1R fusion polypeptide retains the N-terminal 36 amino acid of RBM-6, which includes part of the predicted POZ (poxvirus and zinc finger) domain (POZ domains are known to be involved in protein-protein interactions). The resulting RBM6-CSF1R fusion protein, which comprises 435 amino acids (see panel B of FIG. 1 and FIG. 2 (SEQ ID NO: 1)), retains kinase activity of CSF1R. The exons involved and the fusion junction are shown in FIG. 9 (bottom panel).

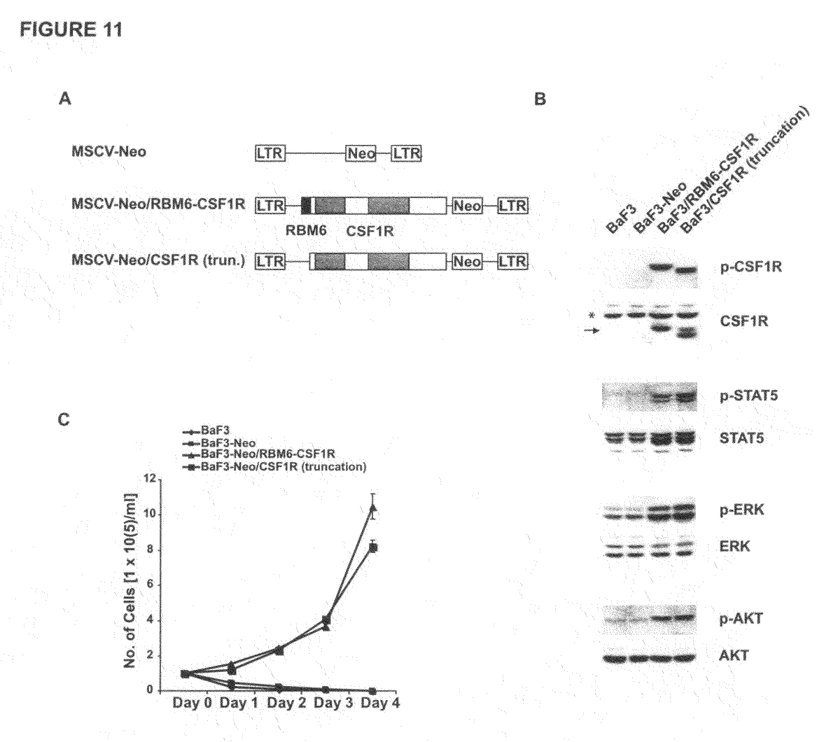

cDNA encoding the fusion protein was prepared and transfected into a murine hematopoietic progenitor cell line (BaF3) to confirm that expression of the mutant CSF1R kinase transforms the cells and drives proliferation and growth (see Example 5; FIG. 11 (panel C)). PCR probes were used to detect the presence of the fusion protein in this AML cell line. cDNA encoding only the truncated mutant CSF1R kinase without the RBM-6 N-terminal moiety was also prepared and used to transform the BaF3 cell line, confirming that the truncated CSF1R kinase retains activity independent of the RBM-6 moiety (see Example 6; FIG. 11 (panel C)).

### B. Isolated Polynucleotides.

The present invention provides, in part, isolated polynucleotides that encode RBM6-CSF1R fusion polypeptides and truncated active CSF1R polypeptides, nucleotide probes that hybridize to such polynucleotides, and methods, vectors, and host cells for utilizing such polynucleotides to produce recombinant fusion polypeptides.

Unless otherwise indicated, all nucleotide sequences determined by sequencing a DNA molecule herein were determined using an automated DNA sequencer (such as the Model 373 from Applied Biosystems, Inc.), and all amino acid sequences of polypeptides encoded by DNA molecules determined herein were determined using an automated peptide sequencer (see Example 4). As is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

Unless otherwise indicated, each nucleotide sequence set forth herein is presented as a sequence of deoxyribonucleotides (abbreviated A, G, C and T). However, by “nucleotide sequence” of a nucleic acid molecule or polynucleotide is intended, for a DNA molecule or polynucleotide, a sequence of deoxyribonucleotides, and for an RNA molecule or polynucleotide, the corresponding sequence of ribonucleotides (A, G, C and U), where each thymidine deoxyribonucleotide (T) in the specified deoxyribonucleotide sequence is replaced by the ribonucleotide uridine (U). For instance, reference to an RNA molecule having the sequence of SEQ ID NO: 2 set forth using deoxyribonucleotide abbreviations is intended to indicate an RNA molecule having a sequence in which each deoxyribonucleotide A, G or C of SEQ ID NO: 2 has been replaced by the corresponding ribonucleotide A, G or C, and each deoxyribonucleotide T has been replaced by a ribonucleotide U.

In one embodiment, the invention provides an isolated polynucleotide comprising a nucleotide sequence at least 95% identical to a sequence selected from the group consisting of:

(a) a nucleotide sequence encoding a RNA Binding Protein-6-Macrophage Colony Stimulating Factor-1 Receptor (RBM6-CSF1R) fusion polypeptide comprising the amino acid sequence of SEQ ID NO: 1;

(b) a nucleotide sequence encoding a RBM6-CSF1R fusion polypeptide, said nucleotide sequence comprising the nucleotide sequence of SEQ ID NO: 2;

(c) a nucleotide sequence encoding a RBM6-CSF1R fusion polypeptide comprising the N-terminal amino acid sequence of RBM-6 (residues 1-36 of SEQ ID NO: 3) and the split kinase domain of CSF1R (residues 582-910 of SEQ ID NO: 5); and

(d) a nucleotide sequence comprising the N-terminal nucleotide sequence of RBM-6 (residues 1-108 of SEQ ID NO: 4) and the split kinase domain nucleotide sequence of CSF1R (residues 1746-2730 of SEQ ID NO: 6);

(e) a nucleotide sequence comprising at least six contiguous nucleotides encompassing the fusion junction (residues 106-111 of SEQ ID NO: 2) of a RBM6-CSF1R fusion polynucleotide;

(f) a nucleotide sequence encoding a polypeptide comprising at least six contiguous amino acids encompassing the fusion junction (residues 36-37 of SEQ ID NO: 1) of a RBM6-CSF1R fusion polypeptide;

(g) a nucleotide sequence encoding a truncated active CSF1R kinase polypeptide comprising residues 574-1123 of SEQ ID NO: 5, but not comprising the extracellular or transmembrane domains of wild type CSF1R;

(h) a nucleotide sequence encoding a truncated active CSF1R kinase polypeptide, said nucleotide sequence comprising nucleotides 1722-2916 of SEQ ID NO: 6, but not encoding the extracellular or transmembrane domains of wild type CSF1R;

(i) a nucleotide sequence encoding a truncated active CSF1R kinase polypeptide comprising the split kinase domain of CSF1R (residues 582-910 of SEQ ID NO: 5) but not comprising the extracellular or transmembrane domains of wild type CSF1R;

(j) a nucleotide sequence comprising up to thirty contiguous nucleotides encompassing the truncation point (residue 1722 of SEQ ID NO: 6) of wild type CSF1R kinase polynucleotide; and

(k) a nucleotide sequence complementary to any of the nucleotide sequences of (a)-(j).

Using the information provided herein, such as the nucleotide sequence in FIG. 2 (SEQ ID NO: 2), a nucleic acid molecule of the present invention encoding a mutant CSF1R polypeptide of the invention may be obtained using standard cloning and screening procedures, such as those for cloning cDNAs using mRNA as starting material. Illustrative of the invention, the RBM6-CSF1R fusion polynucleotide described in FIG. 2 (SEQ ID NO: 2) was isolated from genomic DNA from a human AML cell line (as further described in Example 4 below). The fusion gene can also be identified in genomic DNA or cDNA libraries in other leukemias or cancers in which the RBM6-CSF1R translocation (3p21, 5q33) occurs, or in which a deletion or alternative translocation results in expression of a truncated active CSF1R kinase lacking the extracellular and transmembrane domains of the wild type kinase.

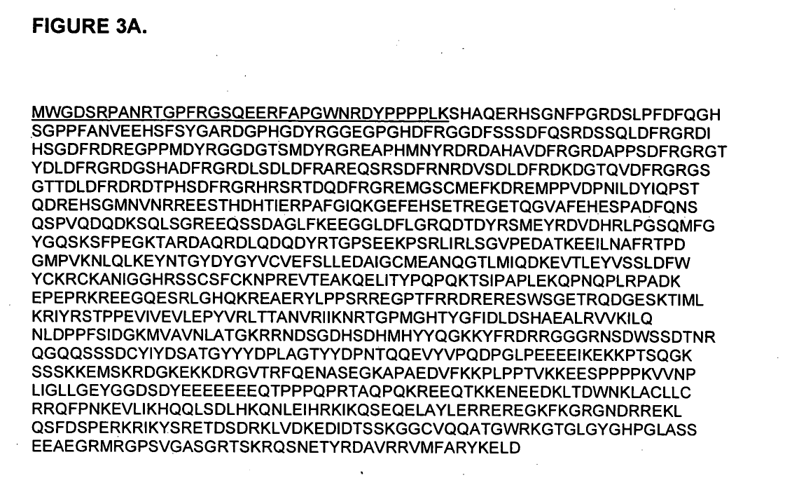

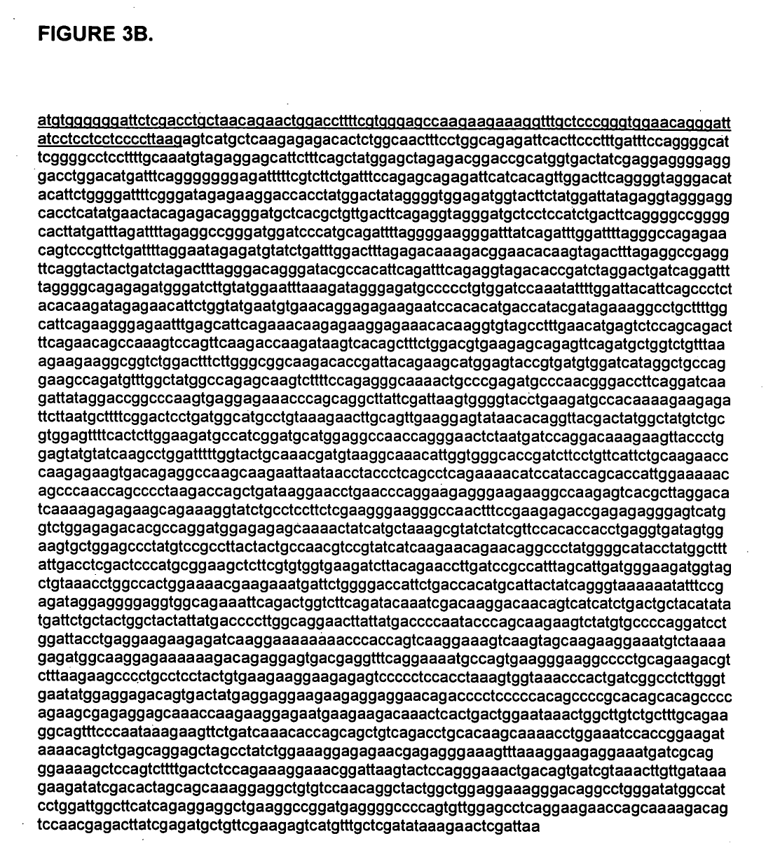

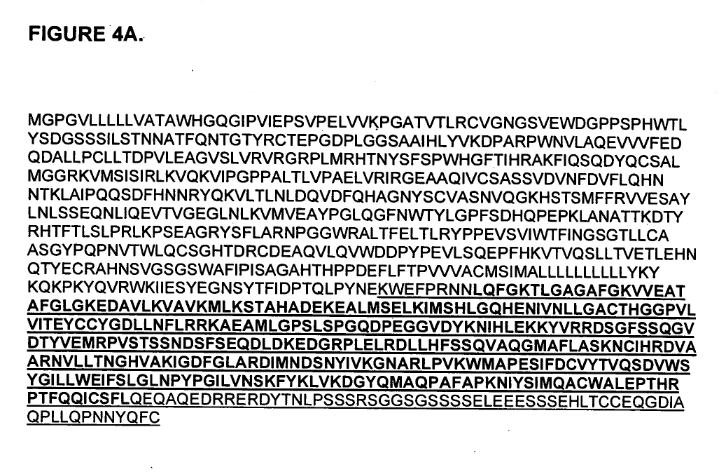

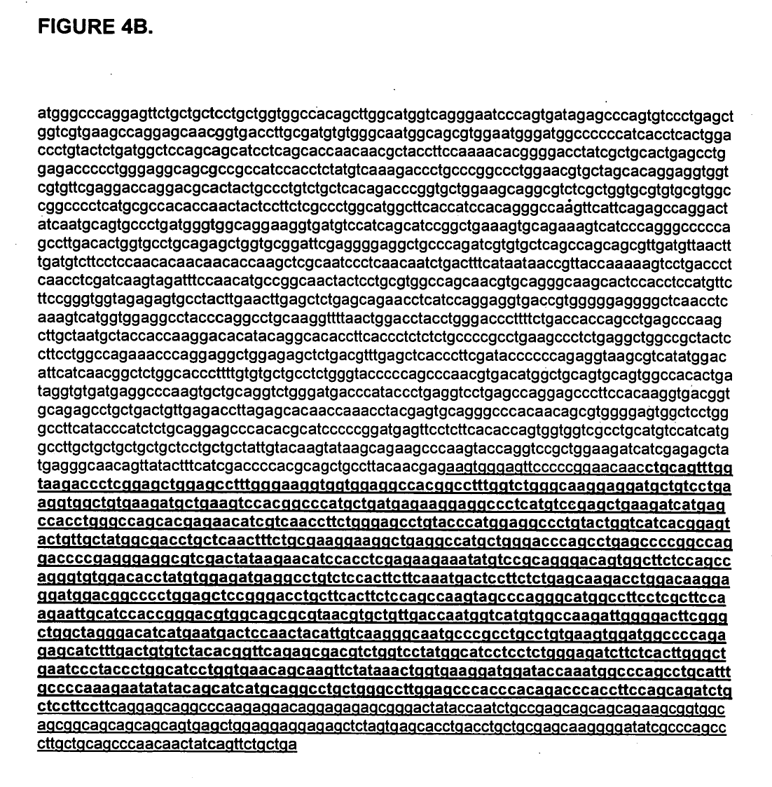

The determined nucleotide sequence of the RBM6-CSF1R translocation gene (SEQ ID NO: 2) encodes a kinase fusion protein of 435 amino acid residues (see FIG. 2 (SEQ ID NO: 1) and FIG. 1). The RBM6-CSF1R fusion polynucleotide comprises the portion of the nucleotide sequence of wild type RBM6 (see FIG. 3 (SEQ ID NO: 4)) that encodes the N-terminus (amino acids 1-36) of that protein with the portion of the nucleotide sequence of wild type CSF1R (see FIG. 4 (SEQ ID NO: 6)) that encodes the juxtamembrane and split kinase domains of that protein. See FIG. 1. The split kinase domain comprises residues 45-373 in the fusion protein (encoded by nucleotides 135-1119 of the fusion polynucleotide).

As indicated, the present invention provides, in part, the mature form of the RBM6-CSF1R fusion protein. According to the signal hypothesis, proteins secreted by mammalian cells have a signal or secretory leader sequence which is cleaved from the mature protein once export of the growing protein chain across the rough endoplasmic reticulum has been initiated. Most mammalian cells and even insect cells cleave secreted proteins with the same specificity. However, in some cases, cleavage of a secreted protein is not entirely uniform, which results in two or more mature species on the protein. Further, it has long been known that the cleavage specificity of a secreted protein is ultimately determined by the primary structure of the complete protein, that is, it is inherent in the amino acid sequence of the polypeptide. Therefore, the present invention provides, in part, nucleotide sequences encoding a mature RBM6-CSF1R fusion polypeptide having the amino acid sequence encoded by the cDNA clone identified as ATCC Deposit No. PTA-7309, which was deposited with the American Type Culture Collection (Manassas, Va., U.S.A.) on Dec. 29, 2005 in accordance with the provisions of the Budapest Treaty.

By the mature RBM6-CSF1R polypeptide having the amino acid sequence encoded by the deposited cDNA clone is meant the mature form of this fusion protein produced by expression in a mammalian cell (e.g., COS cells, as described below) of the complete open reading frame encoded by the human DNA sequence of the deposited clone.

As indicated, polynucleotides of the present invention may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

Isolated polynucleotides of the invention are nucleic acid molecules, DNA or RNA, which have been removed from their native environment. For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

Isolated polynucleotides of the invention include the DNA molecule shown in FIG. 2 (SEQ ID NO: 2), DNA molecules comprising the coding sequence for the mature RBM6-CSF1R fusion protein shown in FIG. 1 (SEQ ID NO: 1), and DNA molecules that comprise a sequence substantially different from those described above but which, due to the degeneracy of the genetic code, still encode a CSF1R mutant polypeptide of the invention. The genetic code is well known in the art, thus, it would be routine for one skilled in the art to generate such degenerate variants.

In another embodiment, the invention provides an isolated polynucleotide encoding the RBM6-CSF1R fusion polypeptide comprising the RBM6-CSF1R translocation nucleotide sequence contained in the above-described deposited cDNA clone. Preferably, such nucleic acid molecule will encode the mature fusion polypeptide encoded by the deposited cDNA clone. In another embodiment, the invention provides an isolated nucleotide sequence encoding a RBM6-CSF1R fusion polypeptide comprising the N-terminal amino acid sequence of RBM-6 (residues 1-36 of SEQ ID NO: 3) and the split kinase domain of CSF1R (residues 582-910 of SEQ ID NO: 5). In one embodiment, the polypeptide comprising the split kinase domain of CSF1R comprises residues 574-972 of SEQ ID NO: 5 (see FIG. 1, panel B). In another embodiment, the aforementioned N-terminal amino acid sequence of RBM-6 and split kinase domain of CSF1R are encoded by nucleotide sequences comprising nucleotides 1-108 of SEQ ID NO: 4 and nucleotides 1746-2730 of SEQ ID NO: 6, respectively.

The present invention also provides in part a truncated active CSF1R kinase comprising the split kinase domains of the wild type protein but lacking the extracellular and transmembrane domains of the wild type protein (see FIG. 1 (panel B)—the truncated kinase comprises residues 574-972 of the wild type CSF1R kinase). In one embodiment, there is provided an isolated polynucleotide comprising a nucleic acid sequence encoding the truncated active CSF1R kinase (e.g. nucleotides 1722-2916 of SEQ ID NO: 6) but not encoding the extracellular and transmembrane domains of the wild type protein. Also provided are nucleotide sequences encoding a truncated active CSF1R kinase polypeptide comprising the split kinase domain of CSF1R (residues 582-910 of SEQ ID NO: 5) but not comprising the extracellular or transmembrane domains of wild type CSF1R. The present invention also provides, in part, nucleotide sequences encoding a truncated active CSF1R polypeptide having the amino acid sequence (residues 574-972 of SEQ ID NO: 1) encoded by the deposited cDNA clone described above (ATCC Deposit No. PTA-7309).

The invention further provides isolated polynucleotides comprising nucleotide sequences having a sequence complementary to one of the mutant CSF1R polynucleotides of the invention. Such isolated molecules, particularly DNA molecules, are useful as probes for gene mapping, by in situ hybridization with chromosomes, and for detecting expression of the RBM6-CSF1R fusion protein or truncated active CSF1R kinase in human tissue, for instance, by Northern blot analysis, as further described in Section F below.

The present invention is further directed to fragments of the isolated nucleic acid molecules described herein. By a fragment of an isolated RBM6-CSF1R polynucleotide or truncated CSF1R polynucleotide of the invention is intended fragments at least about 15 nucleotides, and more preferably at least about 20 nucleotides, still more preferably at least about 30 nucleotides, and even more preferably, at least about 40 nucleotides in length, which are useful as diagnostic probes and primers as discussed herein. Of course, larger fragments of about 50-1500 nucleotides in length are also useful according to the present invention, as are fragments corresponding to most, if not all, of the mutant CSF1R nucleotide sequences of the deposited cDNAs or as shown in FIG. 2 (SEQ ID NO: 2). By a fragment at least 20 nucleotides in length, for example, is intended fragments which include 20 or more contiguous bases from the respective nucleotide sequences from which the fragments are derived.

Generation of such DNA fragments is routine to the skilled artisan, and may be accomplished, by way of example, by restriction endonuclease cleavage or shearing by sonication of DNA obtainable from the deposited cDNA clone or synthesized according to the sequence disclosed herein. Alternatively, such fragments can be directly generated synthetically.

Preferred nucleic acid fragments or probes of the present invention include nucleic acid molecules encoding the fusion junction of the RBM6-CSF1R translocation gene product (see FIG. 1, panel B, and FIG. 9, bottom panel). For example, in certain preferred embodiments, an isolated polynucleotide of the invention comprises a nucleotide sequence/fragment comprising at least six contiguous nucleotides encompassing the fusion junction (nucleotides 106-111 of SEQ ID NO: 2) of a RBM6-CSF1R fusion polynucleotide (see FIG. 9, bottom panel (SEQ ID NO: 8)). In another preferred embodiment, an isolated polynucleotide of the invention comprises a nucleotide sequence/fragment that encodes a polypeptide comprising at least six contiguous amino acids encompassing the fusion junction (residues 36-37 of SEQ ID NO: 1) of a RBM6-CSF1R fusion polypeptide (see FIG. 9, bottom panel (SEQ ID NO: 7)). In another preferred embodiment, an isolated polynucleotide of the invention comprises a nucleotide sequence/fragment comprising up to thirty contiguous nucleotides encompassing the truncation point (residue 1722 of SEQ ID NO: 6) in the wild type CSF1R kinase gene/polynucleotide.

In another aspect, the invention provides an isolated polynucleotide that hybridizes under stringent hybridization conditions to a portion of a mutant CSF1R polynucleotide of the invention as describe herein. By “stringent hybridization conditions” is intended overnight incubation at 42° C. in a solution comprising: 50% formamide, 5×SSC (750 mM NaCl, 75 mM trisodium citrate), 50 mM sodium phosphate (pH 7.6), 5×Denhardt's solution, 10% dextran sulfate, and 20 micrograms/ml denatured, sheared salmon sperm DNA, followed by washing the filters in 0.1×SSC at about 65° C.

By a polynucleotide that hybridizes to a “portion” of a polynucleotide is intended a polynucleotide (either DNA or RNA) hybridizing to at least about 15 nucleotides (nt), and more preferably at least about 20 nt, still more preferably at least about 30 nt, and even more preferably about 30-70 nt of the reference polynucleotide. These are useful as diagnostic probes and primers as discussed above and in more detail below.

Of course, polynucleotides hybridizing to a larger portion of the reference polynucleotide (e.g. the mature RBM6-CSF1R fusion polynucleotide described in FIG. 2 (SEQ ID NO: 2)), for instance, a portion 50-750 nt in length, or even to the entire length of the reference polynucleotide, are also useful as probes according to the present invention, as are polynucleotides corresponding to most, if not all, of the nucleotide sequences of the deposited cDNAs or the nucleotide sequences shown in FIG. 2 (SEQ ID NO: 2), residues 1722-2916 of SEQ ID NO: 2, or FIG. 9 panel B, bottom) (SEQ ID NO: 7).

By a portion of a polynucleotide of “at least 20 nucleotides in length,” for example, is intended 20 or more contiguous nucleotides from the nucleotide sequence of the reference polynucleotide. As indicated, such portions are useful diagnostically either as a probe according to conventional DNA hybridization techniques or as primers for amplification of a target sequence by the polymerase chain reaction (PCR), as described, for instance, in MOLECULAR CLONING, A LABORATORY MANUAL, 2nd. edition, Sambrook, J., Fritsch, E. F. and Maniatis, T., eds., Cold Spring Harbor Laboratory Press, Cold Spring Harbor, N.Y. (1989), the entire disclosure of which is hereby incorporated herein by reference. Of course, a polynucleotide which hybridizes only to a poly A sequence (such as the 3′ terminal poly(A) tract of the RBM6-CSF1R sequence shown in FIG. 2 (SEQ ID NO: 2)) or to a complementary stretch of T (or U) resides, would not be included in a polynucleotide of the invention used to hybridize to a portion of a nucleic acid of the invention, since such a polynucleotide would hybridize to any nucleic acid molecule containing a poly (A) stretch or the complement thereof (e.g., practically any double-stranded cDNA clone).

As indicated, nucleic acid molecules of the present invention, which encode a mutant CSF1R polypeptide of the invention, may include but are not limited to those encoding the amino acid sequence of the mature polypeptide, by itself; the coding sequence for the mature polypeptide and additional sequences, such as those encoding the leader or secretory sequence, such as a pre-, or pro- or pre-pro-protein sequence; the coding sequence of the mature polypeptide, with or without the aforementioned additional coding sequences, together with additional, non-coding sequences, including for example, but not limited to introns and non-coding 5′ and 3′ sequences, such as the transcribed, non-translated sequences that play a role in transcription, mRNA processing, including splicing and polyadenylation signals, for example—ribosome binding and stability of mRNA; an additional coding sequence which codes for additional amino acids, such as those which provide additional functionalities.

Thus, the sequence encoding the polypeptide may be fused to a marker sequence, such as a sequence encoding a peptide that facilitates purification of the fused polypeptide. In certain preferred embodiments of this aspect of the invention, the marker amino acid sequence is a hexa-histidine peptide, such as the tag provided in a pQE vector (Qiagen, Inc.), among others, many of which are commercially available. As described in Gentz et al., Proc. Natl. Acad. Sci. USA 86: 821-824 (1989), for instance, hexa-histidine provides for convenient purification of the fusion protein. The “HA” tag is another peptide useful for purification which corresponds to an epitope derived from the influenza hemagglutinin protein, which has been described by Wilson et al., Cell 37: 767 (1984). As discussed below, other such fusion proteins include the RBM6-CSF1R fusion polypeptide or truncated active CSF1R polypeptide itself fused to Fc at the N- or C-terminus.

The present invention further relates to variants of the nucleic acid molecules of the present invention, which encode portions, analogs or derivatives of a RBM6-CSF1R fusion polypeptide or truncated active CSF1R polypeptide disclosed herein. Variants may occur naturally, such as a natural allelic variant. By an “allelic variant” is intended one of several alternate forms of a gene occupying a given locus on a chromosome of an organism. See, e.g. GENES II, Lewin, B., ed., John Wiley & Sons, New York (1985). Non-naturally occurring variants may be produced using art-known mutagenesis techniques.

Such variants include those produced by nucleotide substitutions, deletions or additions. The substitutions, deletions or additions may involve one or more nucleotides. The variants may be altered in coding regions, non-coding regions, or both. Alterations in the coding regions may produce conservative or non-conservative amino acid substitutions, deletions or additions. Especially preferred among these are silent substitutions, additions and deletions, which do not alter the properties and activities (e.g. kinase activity) of the mutant CSF1R polypeptides disclosed herein. Also especially preferred in this regard are conservative substitutions.

Further embodiments of the invention include isolated polynucleotides comprising a nucleotide sequence at least 90% identical, and more preferably at least 95%, 96%, 97%, 98% or 99% identical, to a mutant CSF1R polynucleotide of the invention (for example, a nucleotide sequence encoding the RBM6-CSF1R fusion polypeptide having the complete amino acid sequence shown in FIG. 2 (SEQ ID NO: 1; or a nucleotide sequence encoding the N-terminal of RBM-6 and the split kinase domains of CSF1R (see FIG. 1, panel B; and FIGS. 3 and 4); or a nucleotide complementary to such exemplary sequences).

By a polynucleotide having a nucleotide sequence at least, for example, 95% “identical” to a reference nucleotide sequence encoding a mutant CSF1R polypeptide is intended that the nucleotide sequence of the polynucleotide is identical to the reference sequence except that the polynucleotide sequence may include up to five point mutations per each 100 nucleotides of the reference nucleotide sequence encoding the mutant CSF1R polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 90%, 95%, 96%, 97%, 98% or 99% identical to, for instance, the nucleotide sequence shown in FIG. 2 (SEQ ID NO: 2) or to the nucleotide sequence of the deposited cDNA clones described above can be determined conventionally using known computer programs such as the Bestfit program (Wisconsin Sequence Analysis Package, Version 8 for Unix, Genetics Computer Group, University Research Park, 575 Science Drive, Madison, Wis. 53711. Bestfit uses the local homology algorithm of Smith and Waterman, Advances in Applied Mathematics 2: 482-489 (1981), to find the best segment of homology between two sequences. When using Bestfit or any other sequence alignment program to determine whether a particular sequence is, for instance, 95% identical to a reference RBM6-CSF1R fusion polynucleotide sequence or truncated CSF1R polynucleotide sequence according to the present invention, the parameters are set, of course, such that the percentage of identity is calculated over the full length of the reference nucleotide sequence and that gaps in homology of up to 5% of the total number of nucleotides in the reference sequence are allowed.

The present invention includes in its scope nucleic acid molecules at least 90%, 95%, 96%, 97%, 98% or 99% identical to the nucleic acid sequence shown in FIG. 2 (SEQ ID NO: 2), or to nucleotides 1722-2916 of SEQ ID NO: 2, or to the nucleic acid sequences of the deposited cDNAs, irrespective of whether they encode a polypeptide having CSF1R kinase activity. This is because even where a particular nucleic acid molecule does not encode a fusion polypeptide having CSF1R kinase activity, one of skill in the art would still know how to use the nucleic acid molecule, for instance, as a hybridization probe or a polymerase chain reaction (PCR) primer. Uses of the nucleic acid molecules of the present invention that do not encode a polypeptide having kinase include, inter alia, (1) isolating the RBM6-CSF1R translocation gene, or truncated CSF1R gene, or allelic variants thereof in a cDNA library; (2) in situ hybridization (e.g., “FISH”) to metaphase chromosomal spreads to provide precise chromosomal location of the RBM6-CSF1R translocation gene or truncated CSF1R gene, as described in Verma et al., HUMAN CHROMOSOMES: A MANUAL OF BASIC TECHNIQUES, Pergamon Press, New York (1988); and Northern Blot analysis for detecting RBM6-CSF1R fusion protein or truncated CSF1R kinase mRNA expression in specific tissues.

Preferred, however, are nucleic acid molecules having sequences at least 95% identical to a mutant CSF1R polypeptide of the invention or to the nucleic acid sequence of the deposited cDNAs which do, in fact, encode a fusion polypeptide having CSF1R kinase activity. Such activity may be similar, but not necessarily identical, to the activity of the RBM6-CSF1R fusion protein and truncated active CSF1R kinase disclosed herein (either the full-length protein, the mature protein, or a protein fragment that retains kinase activity), as measured in a particular biological assay. For example, the kinase activity of CSF1R can be examined by determining its ability to phosphorylate one or more tyrosine containing peptide substrates, for example, “Src-related peptide” (RRLIEDAEYAARG), which is a substrate for many receptor and nonreceptor tyrosine kinases.

Due to the degeneracy of the genetic code, one of ordinary skill in the art will immediately recognize that a large number of the nucleic acid molecules having a sequence at least 90%, 95%, 96%, 97%, 98%, or 99% identical to the nucleic acid sequence of the deposited cDNAs or the nucleic acid sequence shown in FIG. 2 (SEQ ID NO: 2), or nucleotides 1722-2916 of SEQ ID NO: 2, will encode a mutant polypeptide having CSF1R activity. In fact, since degenerate variants of these nucleotide sequences all encode the same polypeptide, this will be clear to the skilled artisan even without performing the above described comparison assay. It will be further recognized in the art that, for such nucleic acid molecules that are not degenerate variants, a reasonable number will also encode a polypeptide that retains CSF1R kinase activity. This is because the skilled artisan is fully aware of amino acid substitutions that are either less likely or not likely to significantly effect protein function (e.g., replacing one aliphatic amino acid with a second aliphatic amino acid).

For example, guidance concerning how to make phenotypically silent amino acid substitutions is provided in Bowie et al., “Deciphering the Message in Protein Sequences: Tolerance to Amino Acid Substitutions,” Science 247:1306-1310 (1990), which describes two main approaches for studying the tolerance of an amino acid sequence to change. The first method relies on the process of evolution, in which mutations are either accepted or rejected by natural selection. The second approach uses genetic engineering to introduce amino acid changes at specific positions of a cloned gene and selections or screens to identify sequences that maintain functionality. These studies have revealed that proteins are surprisingly tolerant of amino acid substitutions. Skilled artisans familiar with such techniques also appreciate which amino acid changes are likely to be permissive at a certain position of the protein. For example, most buried amino acid residues require nonpolar side chains, whereas few features of surface side chains are generally conserved. Other such phenotypically silent substitutions are described in Bowie et al., supra., and the references cited therein.

Methods for DNA sequencing that are well known and generally available in the art may be used to practice any polynucleotide embodiments of the invention. The methods may employ such enzymes as the Klenow fragment of DNA polymerase 1, SEQUENASE® (US Biochemical Corp, Cleveland, Ohio), Taq polymerase (Perkin Elmer), thermostable T7 polymerase (Amersham, Chicago, Ill.), or combinations of recombinant polymerases and proofreading exonucleases such as the ELONGASE Amplification System marketed by Gibco BRL (Gaithersburg, Md.). Preferably, the process is automated with machines such as the Hamilton Micro Lab 2200 (Hamilton, Reno, Nev.), Peltier Thermal Cycler (PTC200; MJ Research, Watertown, Mass.) and the ABI 377 DNA sequencers (Perkin Elmer).

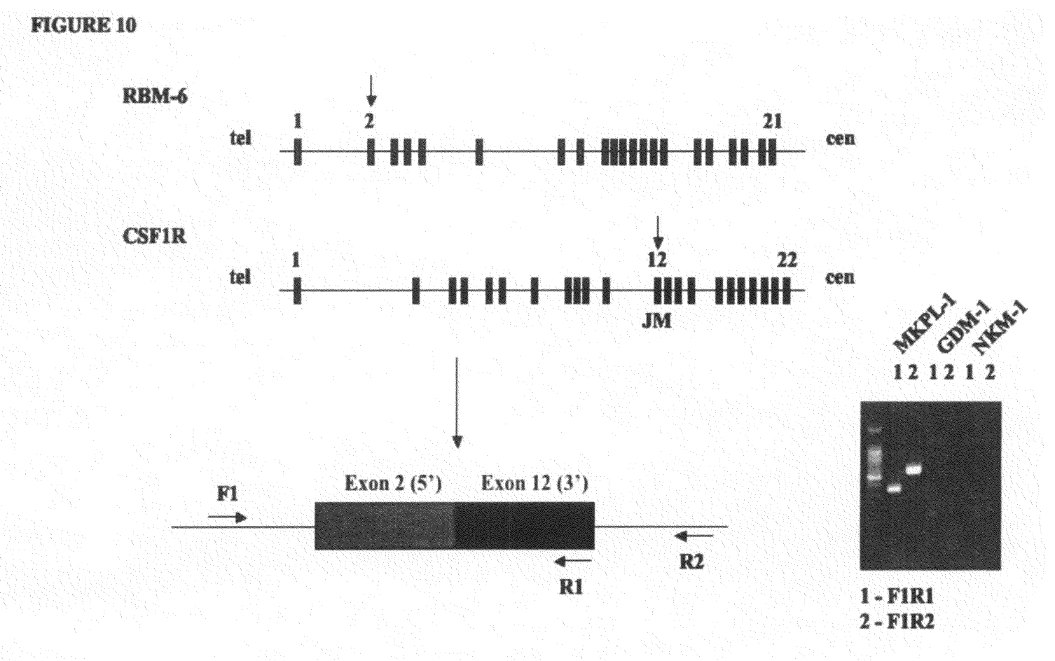

Polynucleotide sequences encoding a mutant CSF1R polypeptide of the invention may be extended utilizing a partial nucleotide sequence and employing various methods known in the art to detect upstream sequences such as promoters and regulatory elements. For example, one method that may be employed, “restriction-site” PCR, uses universal primers to retrieve unknown sequence adjacent to a known locus (Sarkar, G., PCR Methods Applic. 2: 318-322 (1993)). In particular, genomic DNA is first amplified in the presence of primer to linker sequence and a primer specific to the known region. Exemplary primers are those described in Example 4 herein (see also FIG. 10, bottom panel). The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR may also be used to amplify or extend sequences using divergent primers based on a known region (Triglia et al., Nucleic Acids Res. 16: 8186 (1988)). The primers may be designed using OLIGO 4.06 Primer Analysis software (National Biosciences Inc., Plymouth, Minn.), or another appropriate program, to be 22-30 nucleotides in length, to have a GC content of 50% or more, and to anneal to the target sequence at temperatures about 68-72° C. The method uses several restriction enzymes to generate a suitable fragment in the known region of a gene. The fragment is then circularized by intramolecular ligation and used as a PCR template.

Another method which may be used is capture PCR which involves PCR amplification of DNA fragments adjacent to a known sequence in human and yeast artificial chromosome DNA (Lagerstrom et al., PCR Methods Applic. 1: 111-119 (1991)). In this method, multiple restriction enzyme digestions and ligations may also be used to place an engineered double-stranded sequence into an unknown portion of the DNA molecule before performing PCR. Another method which may be used to retrieve unknown sequences is that described in Parker et al., Nucleic Acids Res. 19: 3055-3060 (1991)). Additionally, one may use PCR, nested primers, and PROMOTERFINDER® libraries to walk in genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable, in that they will contain more sequences that contain the 5′ regions of genes. Use of a randomly primed library may be especially preferable for situations in which an oligo d(T) library does not yield a full-length cDNA. Genomic libraries may be useful for extension of sequence into the 5′ and 3′ non-transcribed regulatory regions.

Capillary electrophoresis systems, which are commercially available, may be used to analyze the size or confirm the nucleotide sequence of sequencing or PCR products. In particular, capillary sequencing may employ flowable polymers for electrophoretic separation, four different fluorescent dyes (one for each nucleotide) which are laser activated, and detection of the emitted wavelengths by a charge coupled device camera. Output/light intensity may be converted to electrical signal using appropriate software (e.g. GENOTYPER™ and SEQUENCE NAVIGATOR™, Perkin Elmer) and the entire process from loading of samples to computer analysis and electronic data display may be computer controlled. Capillary electrophoresis is especially preferable for the sequencing of small pieces of DNA that might be present in limited amounts in a particular sample.

### C. Vectors and Host Cells.

The present invention also provides recombinant vectors that comprise an isolated polynucleotide of the present invention, host cells which are genetically engineered with the recombinant vectors, and the production of recombinant RBM6-CSF1R polypeptides, truncated active CSF1R polypeptides, or fragments thereof by recombinant techniques.

Recombinant constructs may be introduced into host cells using well-known techniques such infection, transduction, transfection, transvection, electroporation and transformation. The vector may be, for example, a phage, plasmid, viral or retroviral vector. Retroviral vectors may be replication competent or replication defective. In the latter case, viral propagation generally will occur only in complementing host cells.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

Preferred are vectors comprising cis-acting control regions to the polynucleotide of interest. Appropriate trans-acting factors may be supplied by the host, supplied by a complementing vector or supplied by the vector itself upon introduction into the host.

In certain preferred embodiments in this regard, the vectors provide for specific expression, which may be inducible and/or cell type-specific. Particularly preferred among such vectors are those inducible by environmental factors that are easy to manipulate, such as temperature and nutrient additives.

Expression vectors useful in the present invention include chromosomal-, episomal- and virus-derived vectors, e.g., vectors derived from bacterial plasmids, bacteriophage, yeast episomes, yeast chromosomal elements, viruses such as baculoviruses, papova viruses, vaccinia viruses, adenoviruses, fowl pox viruses, pseudorabies viruses and retroviruses, and vectors derived from combinations thereof, such as cosmids and phagemids.

The DNA insert comprising a RBM6-CSF1R polynucleotide or truncated CSF1R polynucleotide of the invention should be operatively linked to an appropriate promoter, such as the phage lambda PL promoter, the E. coli lac, trp and tac promoters, the SV40 early and late promoters and promoters of retroviral LTRs, to name a few. Other suitable promoters are known to the skilled artisan. The expression constructs will further contain sites for transcription initiation, termination and, in the transcribed region, a ribosome binding site for translation. The coding portion of the mature transcripts expressed by the constructs will preferably include a translation initiating at the beginning and a termination codon (UAA, UGA or UAG) appropriately positioned at the end of the polypeptide to be translated.

As indicated, the expression vectors will preferably include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS and Bowes melanoma cells; and plant cells. Appropriate culture mediums and conditions for the above-described host cells are known in the art.

Among vectors preferred for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Among known bacterial promoters suitable for use in the present invention include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-1 promoter.

In the yeast, Saccharomyces cerevisiae, a number of vectors containing constitutive or inducible promoters such as alpha factor, alcohol oxidase, and PGH may be used. For reviews, see Ausubel et al. (1989) CURRENT PROTOCOLS IN MOLECULAR BIOLOGY, John Wiley & Sons, New York, N.Y., and Grant et al., Methods Enzymol. 153: 516-544 (1997).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., BASIC METHODS IN MOLECULAR BIOLOGY (1986).

Transcription of DNA encoding a RBM6-CSF1R fusion polypeptide of the present invention by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at basepairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide may be expressed in a modified form, such as a fusion protein (e.g. a GST-fusion), and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art. A preferred fusion protein comprises a heterologous region from immunoglobulin that is useful to solubilize proteins.

RBM6-CSF1R polypeptides or truncated active CSF1R polypeptides can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. Polypeptides of the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

Accordingly, in one embodiment, the invention provides a method for producing a recombinant RBM6-CSF1R fusion polypeptide or truncated active CSF1R polypeptide by culturing a recombinant host cell (as described above) under conditions suitable for the expression of the fusion polypeptide and recovering the polypeptide. Culture conditions suitable for the growth of host cells and the expression of recombinant polypeptides from such cells are well known to those of skill in the art. See, e.g., CURRENT PROTOCOLS IN MOLECULAR BIOLOGY, Ausubel F M et al., eds., Volume 2, Chapter 16, Wiley Interscience.

### D. Isolated Polypeptides.

The invention also provides, in part, isolated mutant CSF1R kinase polypeptides and fragments thereof. In one embodiment, the invention provides an isolated polypeptide comprising an amino acid sequence at least 95% identical to a sequence selected from the group consisting of:

(a) an amino acid sequence encoding a RBM6-CSF1R fusion polypeptide comprising the amino acid sequence of SEQ ID NO: 1;

(b) an amino acid sequence encoding a RBM6-CSF1R fusion polypeptide comprising the N-terminal amino acid sequence of RBM-6 (residues 1-36 of SEQ ID NO: 3) and the split kinase domain of CSF1R (residues 582-910 of SEQ ID NO: 5);

(c) an amino acid sequence encoding a polypeptide comprising at least six contiguous amino acids encompassing the fusion junction (residues 36-37 of SEQ ID NO: 1) of a RBM6-CSF1R fusion polypeptide;

(d) an amino acid sequence encoding a truncated active CSF1R kinase polypeptide comprising the amino acid sequence of residues 574-972 of SEQ ID NO: 5, but not comprising the extracellular or transmembrane domains of wild type CSF1R; and

(e) an amino acid sequence encoding a truncated active CSF1R kinase polypeptide comprising the split kinase domain of CSF1R (residues 582-910 of SEQ ID NO: 5), but not comprising the extracellular or transmembrane domains of wild type CSF1R.

In one preferred embodiment, the invention provides an isolated RBM6-CSF1R fusion polypeptide having the amino acid sequence encoded by the first deposited cDNA described above (ATCC Deposit No. PTA-7309). In another preferred embodiment, the invention provides an isolated truncated active CSF1R kinase polypeptide having the amino acid sequence (residues 574-972 of SEQ ID NO: 1) encoded the second deposited cDNA described above. In another preferred embodiment, recombinant mutant CSF1R polypeptides of the invention are provided, which may be produced using a recombinant vector or recombinant host cell as described above.

It will be recognized in the art that some amino acid sequences of a RBM6-CSF1R fusion polypeptide or truncated active CSF1R kinase polypeptide can be varied without significant effect of the structure or function of the mutant protein. If such differences in sequence are contemplated, it should be remembered that there will be critical areas on the protein which determine activity (e.g. the split kinase domains of CSF1R). In general, it is possible to replace residues that form the tertiary structure, provided that residues performing a similar function are used. In other instances, the type of residue may be completely unimportant if the alteration occurs at a non-critical region of the protein.

Thus, the invention further includes variations of a RBM6-CSF1R fusion polypeptide or truncated active CSF1R kinase polypeptide that retain substantial CSF1R kinase activity or that include other regions of RBM6 or CSF1R proteins, such as the protein portions discussed below. Such mutants include deletions, insertions, inversions, repeats, and type substitutions (for example, substituting one hydrophilic residue for another, but not strongly hydrophilic for strongly hydrophobic as a rule). Small changes or such “neutral” amino acid substitutions will generally have little effect on activity.

Typically seen as conservative substitutions are the replacements, one for another, among the aliphatic amino acids Ala, Val, Leu and lie; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Examples of conservative amino acid substitutions known to those skilled in the art are: Aromatic: phenylalanine tryptophan tyrosine; Hydrophobic: leucine isoleucine valine; Polar: glutamine asparagines; Basic: arginine lysine histidine; Acidic: aspartic acid glutamic acid; Small: alanine serine threonine methionine glycine. As indicated in detail above, further guidance concerning which amino acid changes are likely to be phenotypically silent (i.e., are not likely to have a significant deleterious effect on a function) can be found in Bowie et al., Science 247, supra.

The polypeptides of the present invention are preferably provided in an isolated form, and preferably are substantially purified. A recombinantly produced version of a RBM6-CSF1R fusion polypeptide or truncated active CSF1R kinase polypeptide of the invention can be substantially purified by the one-step method described in Smith and Johnson, Gene 67: 3140 (1988).

The polypeptides of the present invention include the RBM6-CSF1R fusion polypeptide of FIG. 2 (SEQ ID NO: 1) (whether or not including a leader sequence), the fusion polypeptide encoded by the deposited cDNA clone (ATCC No. PTA-7309), an amino acid sequence encoding a RBM6-CSF1R fusion polypeptide comprising the N-terminal amino acid sequence of RBM-6 (residues 1-36 of SEQ ID NO: 3) and the split kinase domain of CSF1R (residues 582-910 of SEQ ID NO: 5), and an amino acid sequence encoding a polypeptide comprising at least six contiguous amino acids encompassing the fusion junction (residues 36-37 of SEQ ID NO: 1) of a RBM6-CSF1R fusion polypeptide (see FIG. 9, bottom panel), as well as polypeptides that have at least 90% similarity, preferably at least 95% similarity, and still more preferably at least 96%, 97%, 98% or 99% similarity to those described above.

The polypeptides of the present invention also include an amino acid sequence encoding a truncated active CSF1R kinase polypeptide comprising the amino acid sequence of residues 574-972 of SEQ ID NO: 5, but not comprising the extracellular or transmembrane domains of wild type CSF1R; an amino acid sequence encoding a truncated active CSF1R kinase polypeptide comprising the split kinase domain of CSF1R (residues 582-910 of SEQ ID NO: 5), but not comprising the extracellular or transmembrane domains of wild type CSF1R; and the truncated active CSF1R polypeptide encoded by nucleotides 109-1305 of coding sequence of the deposited cDNA clone (ATCC No. PTA-7309), as well as polypeptides that have at least 90% similarity, more preferably at least 95% similarity, and still more preferably at least 96%, 97%, 98% or 99% similarity to those described above.

By “% similarity” for two polypeptides is intended a similarity score produced by comparing the amino acid sequences of the two polypeptides using the Bestfit program (Wisconsin Sequence Analysis Package, Version 8 for Unix, Genetics Computer Group, University Research Park, 575 Science Drive, Madison, Wis. 53711) and the default settings for determining similarity. Bestfit uses the local homology algorithm of Smith and Waterman (Advances in Applied Mathematics 2: 482-489 (1981)) to find the best segment of similarity between two sequences.

By a polypeptide having an amino acid sequence at least, for example, 95% “identical” to a reference amino acid sequence of a RBM6-CSF1R fusion polypeptide or truncated active CSF1R kinase polypeptide of the invention is intended that the amino acid sequence of the polypeptide is identical to the reference sequence except that the polypeptide sequence may include up to five amino acid alterations per each 100 amino acids of the reference amino acid sequence of the mutant CSF1R polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

A RBM6-CSF1R fusion polypeptide or truncated active CSF1R polypeptide of the present invention may be used as a molecular weight marker on SDS-PAGE gels or on molecular sieve gel filtration columns, for example, using methods well known to those of skill in the art.

As further described in detail below, the polypeptides of the present invention can also be used to generate fusion polypeptide specific reagents, such as polyclonal and monoclonal antibodies, or truncated polypeptide specific reagents, which are useful in assays for detecting mutant CSF1R polypeptide expression as described below, or as agonists and antagonists capable of enhancing or inhibiting the function/activity of the mutant CSF1R protein. Further, such polypeptides can be used in the yeast two-hybrid system to “capture” RBM6-CSF1R fusion polypeptide or truncated active CSF1R kinase polypeptide binding proteins, which are also candidate agonist and antagonist according to the present invention. The yeast two hybrid system is described in Fields and Song, Nature 340: 245-246 (1989).

In another aspect, the invention provides a peptide or polypeptide comprising an epitope-bearing portion of a polypeptide of the invention, for example, an epitope comprising the fusion junction of a RBM6-CSF1R fusion polypeptide (see FIG. 9, bottom panel). The epitope of this polypeptide portion is an immunogenic or antigenic epitope of a polypeptide of the invention. An “immunogenic epitope” is defined as a part of a protein that elicits an antibody response when the whole protein is the immunogen. These immunogenic epitopes are believed to be confined to a few loci on the molecule. On the other hand, a region of a protein molecule to which an antibody can bind is defined as an “antigenic epitope.” The number of immunogenic epitopes of a protein generally is less than the number of antigenic epitopes. See, for instance, Geysen et al., Proc. Natl. Acad. Sci. USA 81:3998-4002 (1983). The production of fusion polypeptide-specific antibodies of the invention is described in further detail below.

The antibodies raised by antigenic epitope-bearing peptides or polypeptides are useful to detect a mimicked protein, and antibodies to different peptides may be used for tracking the fate of various regions of a protein precursor which undergoes post-translational processing. The peptides and anti-peptide antibodies may be used in a variety of qualitative or quantitative assays for the mimicked protein, for instance in competition assays since it has been shown that even short peptides (e.g., about 9 amino acids) can bind and displace the larger peptides in immunoprecipitation assays. See, for instance, Wilson et al., Cell 37: 767-778 (1984) at 777. The anti-peptide antibodies of the invention also are useful for purification of the mimicked protein, for instance, by adsorption chromatography using methods well known in the art. Immunological assay formats are described in further detail below.

Recombinant mutant CSF1R polypeptides are also within the scope of the present invention, and may be producing using polynucleotides of the invention, as described in Section B above. For example, the invention provides, in part, a method for producing a recombinant RBM6-CSF1R fusion polypeptide or truncated active CSF1R kinase polypeptide by culturing a recombinant host cell (as described above) under conditions suitable for the expression of the fusion polypeptide and recovering the polypeptide. Culture conditions suitable for the growth of host cells and the expression of recombinant polypeptides from such cells are well known to those of skill in the art.

### E. Mutant-Specific Reagents

Mutant CSF1R polypeptide-specific reagents useful in the practice of the disclosed methods include, among others, fusion polypeptide specific antibodies and AQUA peptides (heavy-isotope labeled peptides) corresponding to, and suitable for detection and quantification of, RBM6-CSF1R fusion polypeptide expression in a biological sample. Also useful are truncation-specific reagents, such as antibodies or AQUA peptides, suitable for detecting the presence or absence of a truncated active CSF1R kinase polypeptide of the invention. A fusion polypeptide-specific reagent is any reagent, biological or chemical, capable of specifically binding to, detecting and/or quantifying the presence/level of expressed RBM6-CSF1R fusion polypeptide in a biological sample. The term includes, but is not limited to, the preferred antibody and AQUA peptide reagents discussed below, and equivalent reagents are within the scope of the present invention.

### Antibodies.

Reagents suitable for use in practice of the methods of the invention include a RBM6-CSF1R fusion polypeptide-specific antibody. A fusion-specific antibody of the invention is an isolated antibody or antibodies that specifically bind(s) a RBM6-CSF1R fusion polypeptide of the invention (e.g. SEQ ID NO: 1) but does not substantially bind either wild type RBM6 or wild type CSF1R. Other suitable reagents include epitope-specific antibodies that specifically bind to an epitope in the extracelluar domain of wild type CSF1R protein sequence (which domain is not present in the truncated, active CSF1R kinase disclosed herein), and are therefore capable of detecting the presence (or absence) of wild type CSF1R in a sample.

Human RBM6-CSF1R fusion polypeptide-specific antibodies may also bind to highly homologous and equivalent epitopic peptide sequences in other mammalian species, for example murine or rabbit, and vice versa. Antibodies useful in practicing the methods of the invention include (a) monoclonal antibodies, (b) purified polyclonal antibodies that specifically bind to the target polypeptide (e.g. the fusion junction of RBM6-CSF1R fusion polypeptide (see FIG. 9, bottom panel), (c) antibodies as described in (a)-(b) above that bind equivalent and highly homologous epitopes or phosphorylation sites in other non-human species (e.g. mouse, rat), and (d) fragments of (a)-(c) above that bind to the antigen (or more preferably the epitope) bound by the exemplary antibodies disclosed herein.

The term “antibody” or “antibodies” as used herein refers to all types of immunoglobulins, including IgG, IgM, IgA, IgD, and IgE. The antibodies may be monoclonal or polyclonal and may be of any species of origin, including (for example) mouse, rat, rabbit, horse, or human, or may be chimeric antibodies. See, e.g., M. Walker et al., Molec. Immunol. 26: 403-11 (1989); Morrision et al., Proc. Nat'l. Acad. Sci. 81: 6851 (1984); Neuberger et al., Nature 312: 604 (1984)). The antibodies may be recombinant monoclonal antibodies produced according to the methods disclosed in U.S. Pat. No. 4,474,893 (Reading) or U.S. Pat. No. 4,816,567 (Cabilly et al.) The antibodies may also be chemically constructed specific antibodies made according to the method disclosed in U.S. Pat. No. 4,676,980 (Segel et al.)

The preferred epitopic site of a RBM6-CSF1R fusion polypeptide specific antibody of the invention is a peptide fragment consisting essentially of about 11 to 17 amino acids of the human RBM6-CSF1R fusion polypeptide sequence (SEQ ID NO: 1) which fragment encompasses the fusion junction (which occurs at residues 36-37 in the fusion protein (see FIG. 1 (bottom panel) and FIG. 9 (bottom panel)). It will be appreciated that antibodies that specifically binding shorter or longer peptides/epitopes encompassing the fusion junction of the RBM6-CSF1R fusion polypeptide are within the scope of the present invention.

The invention is not limited to use of antibodies, but includes equivalent molecules, such as protein binding domains or nucleic acid aptamers, which bind, in a fusion-protein or truncated-protein specific manner, to essentially the same epitope to which a RBM6-CSF1R fusion polypeptide-specific antibody or CSF1R truncation point epitope-specific antibody useful in the methods of the invention binds. See, e.g., Neuberger et al., Nature 312:604 (1984). Such equivalent non-antibody reagents may be suitably employed in the methods of the invention further described below.

Polyclonal antibodies useful in practicing the methods of the invention may be produced according to standard techniques by immunizing a suitable animal (e.g., rabbit, goat, etc.) with an antigen encompassing a desired fusion-protein specific epitope (e.g. the fusion junction (see FIG. 9, bottom panel), collecting immune serum from the animal, and separating the polyclonal antibodies from the immune serum, and purifying polyclonal antibodies having the desired specificity, in accordance with known procedures. The antigen may be a synthetic peptide antigen comprising the desired epitopic sequence, selected and constructed in accordance with well-known techniques. See, e.g., ANTIBODIES: A LABORATORY MANUAL, Chapter 5, p. 75-76, Harlow & Lane Eds., Cold Spring Harbor Laboratory (1988); Czernik, Methods In Enzymology, 201: 264-283 (1991); Merrifield, J. Am. Chem. Soc. 85:21-49 (1962)). Polyclonal antibodies produced as described herein may be screened and isolated as further described below.

Monoclonal antibodies may also be beneficially employed in the methods of the invention, and may be produced in hybridoma cell lines according to the well-known technique of Kohler and Milstein. Nature 265:495-97 (1975); Kohler and Milstein, Eur. J. Immunol. 6: 511 (1976); see also, CURRENT PROTOCOLS IN MOLECULAR BIOLOGY, Ausubel et al. Eds. (1989). Monoclonal antibodies so produced are highly specific, and improve the selectivity and specificity of assay methods provided by the invention. For example, a solution containing the appropriate antigen (e.g. a synthetic peptide comprising the fusion junction of RBM6-CSF1R fusion polypeptide) may be injected into a mouse and, after a sufficient time (in keeping with conventional techniques), the mouse sacrificed and spleen cells obtained. The spleen cells are then immortalized by fusing them with myeloma cells, typically in the presence of polyethylene glycol, to produce hybridoma cells. Rabbit fusion hybridomas, for example, may be produced as described in U.S. Pat. No. 5,675,063, K. Knight, Issued Oct. 7, 1997. The hybridoma cells are then grown in a suitable selection media, such as hypoxanthine-aminopterin-thymidine (HAT), and the supernatant screened for monoclonal antibodies having the desired specificity, as described below. The secreted antibody may be recovered from tissue culture supernatant by conventional methods such as precipitation, ion exchange or affinity chromatography, or the like.

Monoclonal Fab fragments may also be produced in Escherichia coli by recombinant techniques known to those skilled in the art. See, e.g., W. Huse, Science 246: 1275-81 (1989); Mullinax et al., Proc. Nat'l Acad. Sci. 87: 8095 (1990). If monoclonal antibodies of one isotype are preferred for a particular application, particular isotypes can be prepared directly, by selecting from the initial fusion, or prepared secondarily, from a parental hybridoma secreting a monoclonal antibody of different isotype by using the sib selection technique to isolate class-switch variants (Steplewski, et al., Proc. Nat'l. Acad. Sci., 82: 8653 (1985); Spira et al., J. Immunol. Methods, 74: 307 (1984)). The antigen combining site of the monoclonal antibody can be cloned by PCR and single-chain antibodies produced as phage-displayed recombinant antibodies or soluble antibodies in E. coli (see, e.g., ANTIBODY ENGINEERING PROTOCOLS, 1995, Humana Press, Sudhir Paul editor.)

Further still, U.S. Pat. No. 5,194,392, Geysen (1990) describes a general method of detecting or determining the sequence of monomers (amino acids or other compounds) which is a topological equivalent of the epitope (i.e., a “mimotope”) which is complementary to a particular paratope (antigen binding site) of an antibody of interest. More generally, this method involves detecting or determining a sequence of monomers which is a topographical equivalent of a ligand which is complementary to the ligand binding site of a particular receptor of interest. Similarly, U.S. Pat. No. 5,480,971, Houghten et al. (1996) discloses linear C₁—C-alkyl peralkylated oligopeptides and sets and libraries of such peptides, as well as methods for using such oligopeptide sets and libraries for determining the sequence of a peralkylated oligopeptide that preferentially binds to an acceptor molecule of interest. Thus, non-peptide analogs of the epitope-bearing peptides of the invention also can be made routinely by these methods.

Antibodies useful in the methods of the invention, whether polyclonal or monoclonal, may be screened for epitope and fusion protein specificity according to standard techniques. See, e.g. Czernik et al., Methods in Enzymology, 201: 264-283 (1991). For example, the antibodies may be screened against a peptide library by ELISA to ensure specificity for both the desired antigen and, if desired, for reactivity only with a RBM6-CSF1R fusion polypeptide of the invention and not with wild type RBM6 or wild type CSF1R. The antibodies may also be tested by Western blotting against cell preparations containing target protein to confirm reactivity with the only the desired target and to ensure no appreciable binding to other fusion proteins involving CSF1R. The production, screening, and use of fusion protein-specific antibodies is known to those of skill in the art, and has been described. See, e.g., U.S. Patent Publication No. 20050214301, Wetzel et al., Sep. 29, 2005.

Fusion polypeptide-specific antibodies useful in the methods of the invention may exhibit some limited cross-reactivity with similar fusion epitopes in other fusion proteins or with the epitopes in wild type RBM6 and wild type CSF1R that form the fusion junction. This is not unexpected as most antibodies exhibit some degree of cross-reactivity, and anti-peptide antibodies will often cross-react with epitopes having high homology or identity to the immunizing peptide. See, e.g., Czernik, supra. Cross-reactivity with other fusion proteins is readily characterized by Western blotting alongside markers of known molecular weight. Amino acid sequences of cross-reacting proteins may be examined to identify sites highly homologous or identical to the RBM6-CSF1R fusion polypeptide sequence to which the antibody binds. Undesirable cross-reactivity can be removed by negative selection using antibody purification on peptide columns (e.g. selecting out antibodies that bind either wild type RBM6 and/or wild type CSF1R).

RBM6-CSF1R fusion polypeptide-specific antibodies of the invention that are useful in practicing the methods disclosed herein are ideally specific for human fusion polypeptide, but are not limited only to binding the human species, per se. The invention includes the production and use of antibodies that also bind conserved and highly homologous or identical epitopes in other mammalian species (e.g. mouse, rat, monkey). Highly homologous or identical sequences in other species can readily be identified by standard sequence comparisons, such as using BLAST, with the human RBM6-CSF1R fusion polypeptide sequence disclosed herein (SEQ ID NO: 1).

Antibodies employed in the methods of the invention may be further characterized by, and validated for, use in a particular assay format, for example FC, IHC, and/or ICC. The use of RBM6-CSF1R fusion polypeptide-specific antibodies in such methods is further described in Section F below. Antibodies may also be advantageously conjugated to fluorescent dyes (e.g. Alexa488, PE), or labels such as quantum dots, for use in multi-parametric analyses along with other signal transduction (phospho-AKT, phospho-Erk 1/2) and/or cell marker (cytokeratin) antibodies, as further described in Section F below.

In practicing the methods of the invention, the expression and/or activity of wild type RBM6 and/or wild type CSF1R in a given biological sample may also be advantageously examined using antibodies (either phospho-specific or total) for these wild type proteins. For example, CSF receptor phosphorylation-site specific antibodies are commercially available (see CELL SIGNALING TECHNOLOGY, INC., Beverly Mass., 2005/06 Catalogue, #'s 3151, 3155, and 3154; and Upstate Biotechnology, 2006 Catalogue, #06-457). Such antibodies may also be produced according to standard methods, as described above. The amino acid sequences of both human RBM-6 and CSF1R are published (see FIGS. 3 and 4, and referenced SwissProt Accession Nos.), as are the sequences of these proteins from other species.

Detection of wild type RBM-6 and wild type CSF1R expression and/or activation, along with RBM6-CSF1R fusion polypeptide expression, in a biological sample (e.g. a tumor sample) can provide information on whether the fusion protein alone is driving the tumor, or whether wild type CSF1R is also activated and driving the tumor. Such information is clinically useful in assessing whether targeting the fusion protein or the wild type protein(s), or both, or is likely to be most beneficial in inhibiting progression of the tumor, and in selecting an appropriate therapeutic or combination thereof. Antibodies specific for the wild type CSF1R kinase extracellular domain, which is not present in the truncated active CSF1R kinase disclosed herein, may be particularly useful for determining the presence/absence of the mutant CSF1R kinase.

It will be understood that more than one antibody may be used in the practice of the above-described methods. For example, one or more RBM6-CSF1R fusion polypeptide-specific antibodies together with one or more antibodies specific for another kinase, receptor, or kinase substrate that is suspected of being, or potentially is, activated in a cancer in which RBM6-CSF1R fusion polypeptide is expressed may be simultaneously employed to detect the activity of such other signaling molecules in a biological sample comprising cells from such cancer.

Those of skill in the art will appreciate that RBM6-CSF1R fusion polypeptides of the present invention and the fusion junction epitope-bearing fragments thereof described above can be combined with parts of the constant domain of immunoglobulins (IgG), resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EPA 394,827; Traunecker et al., Nature 331: 84-86 (1988)). Fusion proteins that have a disulfide-linked dimeric structure due to the IgG part can also be more efficient in binding and neutralizing other molecules than the monomeric RBM6-CSF1R fusion polypeptide alone (Fountoulakis et al., J Biochem 270: 3958-3964 (1995)).

### Heavy-Isotope Labeled Peptides (AQUA Peptides).

RBM6-CSF1R fusion polypeptide-specific reagents useful in the practice of the disclosed methods may also comprise heavy-isotope labeled peptides suitable for the absolute quantification of expressed RBM6-CSF1R fusion polypeptide or truncated active CSF1R kinase polypeptide in a biological sample. The production and use of AQUA peptides for the absolute quantification of proteins (AQUA) in complex mixtures has been described. See WO/03016861, “Absolute Quantification of Proteins and Modified Forms Thereof by Multistage Mass Spectrometry,” Gygi et al. and also Gerber et al. Proc. Natl. Acad. Sci. U.S.A. 100: 6940-5 (2003) (the teachings of which are hereby incorporated herein by reference, in their entirety).

The AQUA methodology employs the introduction of a known quantity of at least one heavy-isotope labeled peptide standard (which has a unique signature detectable by LC-SRM chromatography) into a digested biological sample in order to determine, by comparison to the peptide standard, the absolute quantity of a peptide with the same sequence and protein modification in the biological sample. Briefly, the AQUA methodology has two stages: peptide internal standard selection and validation and method development; and implementation using validated peptide internal standards to detect and quantify a target protein in sample. The method is a powerful technique for detecting and quantifying a given peptide/protein within a complex biological mixture, such as a cell lysate, and may be employed, e.g., to quantify change in protein phosphorylation as a result of drug treatment, or to quantify differences in the level of a protein in different biological states.

Generally, to develop a suitable internal standard, a particular peptide (or modified peptide) within a target protein sequence is chosen based on its amino acid sequence and the particular protease to be used to digest. The peptide is then generated by solid-phase peptide synthesis such that one residue is replaced with that same residue containing stable isotopes (¹³C, ¹⁵N). The result is a peptide that is chemically identical to its native counterpart formed by proteolysis, but is easily distinguishable by MS via a 7-Da mass shift. The newly synthesized AQUA internal standard peptide is then evaluated by LC-MS/MS. This process provides qualitative information about peptide retention by reverse-phase chromatography, ionization efficiency, and fragmentation via collision-induced dissociation. Informative and abundant fragment ions for sets of native and internal standard peptides are chosen and then specifically monitored in rapid succession as a function of chromatographic retention to form a selected reaction monitoring (LC-SRM) method based on the unique profile of the peptide standard.

The second stage of the AQUA strategy is its implementation to measure the amount of a protein or modified protein from complex mixtures. Whole cell lysates are typically fractionated by SDS-PAGE gel electrophoresis, and regions of the gel consistent with protein migration are excised. This process is followed by in-gel proteolysis in the presence of the AQUA peptides and LC-SRM analysis. (See Gerber et al. supra.) AQUA peptides are spiked in to the complex peptide mixture obtained by digestion of the whole cell lysate with a proteolytic enzyme and subjected to immunoaffinity purification as described above. The retention time and fragmentation pattern of the native peptide formed by digestion (e.g. trypsinization) is identical to that of the AQUA internal standard peptide determined previously; thus, LC-MS/MS analysis using an SRM experiment results in the highly specific and sensitive measurement of both internal standard and analyte directly from extremely complex peptide mixtures.

Since an absolute amount of the AQUA peptide is added (e.g. 250 fmol), the ratio of the areas under the curve can be used to determine the precise expression levels of a protein or phosphorylated form of a protein in the original cell lysate. In addition, the internal standard is present during in-gel digestion as native peptides are formed, such that peptide extraction efficiency from gel pieces, absolute losses during sample handling (including vacuum centrifugation), and variability during introduction into the LC-MS system do not affect the determined ratio of native and AQUA peptide abundances.

An AQUA peptide standard is developed for a known sequence previously identified by the IAP-LC-MS/MS method within in a target protein. If the site is modified, one AQUA peptide incorporating the modified form of the particular residue within the site may be developed, and a second AQUA peptide incorporating the unmodified form of the residue developed. In this way, the two standards may be used to detect and quantify both the modified an unmodified forms of the site in a biological sample.

Peptide internal standards may also be generated by examining the primary amino acid sequence of a protein and determining the boundaries of peptides produced by protease cleavage. Alternatively, a protein may actually be digested with a protease and a particular peptide fragment produced can then sequenced. Suitable proteases include, but are not limited to, serine proteases (e.g. trypsin, hepsin), metallo proteases (e.g. PUMP1), chymotrypsin, cathepsin, pepsin, thermolysin, carboxypeptidases, etc.

A peptide sequence within a target protein is selected according to one or more criteria to optimize the use of the peptide as an internal standard. Preferably, the size of the peptide is selected to minimize the chances that the peptide sequence will be repeated elsewhere in other non-target proteins. Thus, a peptide is preferably at least about 6 amino acids. The size of the peptide is also optimized to maximize ionization frequency. Thus, peptides longer than about 20 amino acids are not preferred. The preferred ranged is about 7 to 15 amino acids. A peptide sequence is also selected that is not likely to be chemically reactive during mass spectrometry, thus sequences comprising cysteine, tryptophan, or methionine are avoided.

A peptide sequence that does not include a modified region of the target region may be selected so that the peptide internal standard can be used to determine the quantity of all forms of the protein. Alternatively, a peptide internal standard encompassing a modified amino acid may be desirable to detect and quantify only the modified form of the target protein. Peptide standards for both modified and unmodified regions can be used together, to determine the extent of a modification in a particular sample (i.e. to determine what fraction of the total amount of protein is represented by the modified form). For example, peptide standards for both the phosphorylated and unphosphorylated form of a protein known to be phosphorylated at a particular site can be used to quantify the amount of phosphorylated form in a sample.

The peptide is labeled using one or more labeled amino acids (i.e. the label is an actual part of the peptide) or less preferably, labels may be attached after synthesis according to standard methods. Preferably, the label is a mass-altering label selected based on the following considerations: The mass should be unique to shift fragments masses produced by MS analysis to regions of the spectrum with low background; the ion mass signature component is the portion of the labeling moiety that preferably exhibits a unique ion mass signature in MS analysis; the sum of the masses of the constituent atoms of the label is preferably uniquely different than the fragments of all the possible amino acids. As a result, the labeled amino acids and peptides are readily distinguished from unlabeled ones by the ion/mass pattern in the resulting mass spectrum. Preferably, the ion mass signature component imparts a mass to a protein fragment that does not match the residue mass for any of the 20 natural amino acids.

The label should be robust under the fragmentation conditions of MS and not undergo unfavorable fragmentation. Labeling chemistry should be efficient under a range of conditions, particularly denaturing conditions, and the labeled tag preferably remains soluble in the MS buffer system of choice. The label preferably does not suppress the ionization efficiency of the protein and is not chemically reactive. The label may contain a mixture of two or more isotopically distinct species to generate a unique mass spectrometric pattern at each labeled fragment position. Stable isotopes, such as ²H, ¹³C, ¹⁵N, ¹⁷O, ¹⁸O, or ³⁴S, are among preferred labels. Pairs of peptide internal standards that incorporate a different isotope label may also be prepared. Preferred amino acid residues into which a heavy isotope label may be incorporated include leucine, proline, valine, and phenylalanine.

Peptide internal standards are characterized according to their mass-to-charge (m/z) ratio, and preferably, also according to their retention time on a chromatographic column (e.g. an HPLC column). Internal standards that co-elute with unlabeled peptides of identical sequence are selected as optimal internal standards. The internal standard is then analyzed by fragmenting the peptide by any suitable means, for example by collision-induced dissociation (CID) using, e.g., argon or helium as a collision gas. The fragments are then analyzed, for example by multi-stage mass spectrometry (MSⁿ) to obtain a fragment ion spectrum, to obtain a peptide fragmentation signature. Preferably, peptide fragments have significant differences in m/z ratios to enable peaks corresponding to each fragment to be well separated, and a signature is that is unique for the target peptide is obtained. If a suitable fragment signature is not obtained at the first stage, additional stages of MS are performed until a unique signature is obtained.

Fragment ions in the MS/MS and MS³ spectra are typically highly specific for the peptide of interest, and, in conjunction with LC methods, allow a highly selective means of detecting and quantifying a target peptide/protein in a complex protein mixture, such as a cell lysate, containing many thousands or tens of thousands of proteins. Any biological sample potentially containing a target protein/peptide of interest may be assayed. Crude or partially purified cell extracts are preferably employed. Generally, the sample has at least 0.01 mg of protein, typically a concentration of 0.1-10 mg/mL, and may be adjusted to a desired buffer concentration and pH.

A known amount of a labeled peptide internal standard, preferably about 10 femtomoles, corresponding to a target protein to be detected/quantified is then added to a biological sample, such as a cell lysate. The spiked sample is then digested with one or more protease(s) for a suitable time period to allow digestion. A separation is then performed (e.g. by HPLC, reverse-phase HPLC, capillary electrophoresis, ion exchange chromatography, etc.) to isolate the labeled internal standard and its corresponding target peptide from other peptides in the sample. Microcapillary LC is a preferred method.

Each isolated peptide is then examined by monitoring of a selected reaction in the MS. This involves using the prior knowledge gained by the characterization of the peptide internal standard and then requiring the MS to continuously monitor a specific ion in the MS/MS or MSⁿ spectrum for both the peptide of interest and the internal standard. After elution, the area under the curve (AUC) for both peptide standard and target peptide peaks are calculated. The ratio of the two areas provides the absolute quantification that can be normalized for the number of cells used in the analysis and the protein's molecular weight, to provide the precise number of copies of the protein per cell. Further details of the AQUA methodology are described in Gygi et al., and Gerber et al. supra.

AQUA internal peptide standards (heavy-isotope labeled peptides) may desirably be produced, as described above, to detect any quantify any unique site (e.g. the fusion junction within RBM6-CSF1R fusion polypeptide) within a mutant CSF1R polypeptide of the invention. For example, an AQUA phosphopeptide may be prepared that corresponds to the fusion junction sequence of RBM6-CSF1R fusion polypeptide (see FIG. 9 (bottom panel)). Peptide standards for may be produced for the RBM6-CSF1R fusion junction and such standards employed in the AQUA methodology to detect and quantify the fusion junction (i.e. the presence of RBM6-CSF1R fusion polypeptide) in a biological sample.

For example, an exemplary AQUA peptide of the invention comprises the amino acid sequence PLKKWE (see FIG. 9, bottom panel), which corresponds to the three amino acids immediately flanking each side of the fusion junction in RBM6-CSF1R fusion polypeptide (see SEQ ID NO: 7). It will be appreciated that larger AQUA peptides comprising the fusion junction sequence (and additional residues downstream or upstream of it) may also be constructed. Similarly, a smaller AQUA peptide comprising less than all of the residues of such sequence (but still comprising the point of fusion junction itself) may alternatively be constructed. Such larger or shorter AQUA peptides are within the scope of the present invention, and the selection and production of preferred AQUA peptides may be carried out as described above (see Gygi et al., Gerber et al., supra.).

### Nucleic Acid Probes.

Fusion-specific reagents provided by the invention also include nucleic acid probes and primers suitable for detection of a RBM6-CSF1R polynucleotide or truncated CSF1R kinase polynucleotide, as described in detail in Section B above. The specific use of such probes in assays such as fluorescence in-situ hybridization (FISH) or polymerase chain reaction (PCR) amplification is described in Section F below.

### F. Diagnostic Applications & Assay Formats.

The methods of the invention may be carried out in a variety of different assay formats known to those of skill in the art.

### Immunoassays.

Immunoassays useful in the practice of the methods of the invention may be homogenous immunoassays or heterogeneous immunoassays. In a homogeneous assay the immunological reaction usually involves a mutant CSF1R kinase polypeptide-specific reagent (e.g. a RBM6-CSF1R fusion polypeptide-specific antibody), a labeled analyte, and the biological sample of interest. The signal arising from the label is modified, directly or indirectly, upon the binding of the antibody to the labeled analyte. Both the immunological reaction and detection of the extent thereof are carried out in a homogeneous solution. Immunochemical labels that may be employed include free radicals, radio-isotopes, fluorescent dyes, enzymes, bacteriophages, coenzymes, and so forth. Semi-conductor nanocrystal labels, or “quantum dots”, may also be advantageously employed, and their preparation and use has been well described. See generally, K. Barovsky, Nanotech. Law & Bus. 1(2): Article 14 (2004) and patents cited therein.

In a heterogeneous assay approach, the reagents are usually the biological sample, a mutant CSF1R kinase polypeptide-specific reagent (e.g., a RBM6-CSF1R fusion-specific antibody), and suitable means for producing a detectable signal. Biological samples as further described below may be used. The antibody is generally immobilized on a support, such as a bead, plate or slide, and contacted with the sample suspected of containing the antigen in a liquid phase. The support is then separated from the liquid phase and either the support phase or the liquid phase is examined for a detectable signal employing means for producing such signal. The signal is related to the presence of the analyte in the biological sample. Means for producing a detectable signal include the use of radioactive labels, fluorescent labels, enzyme labels, quantum dots, and so forth. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are the radioimmunoassay, immunofluorescence methods, enzyme-linked immunoassays, and the like.

Immunoassay formats and variations thereof, which may be useful for carrying out the methods disclosed herein, are well known in the art. See generally E. Maggio, Enzyme-Immunoassay, (1980) (CRC Press, Inc., Boca Raton, Fla.); see also, e.g., U.S. Pat. No. 4,727,022 (Skold et al., “Methods for Modulating Ligand-Receptor Interactions and their Application”); U.S. Pat. No. 4,659,678 (Forrest et al., “Immunoassay of Antigens”); U.S. Pat. No. 4,376,110 (David et al., “Immunometric Assays Using Monoclonal Antibodies”). Conditions suitable for the formation of reagent-antibody complexes are well known to those of skill in the art. See id. RBM6-CSF1R fusion polypeptide-specific monoclonal antibodies may be used in a “two-site” or “sandwich” assay, with a single hybridoma cell line serving as a source for both the labeled monoclonal antibody and the bound monoclonal antibody. Such assays are described in U.S. Pat. No. 4,376,110. The concentration of detectable reagent should be sufficient such that the binding of RBM6-CSF1R fusion polypeptide is detectable compared to background.

Antibodies useful in the practice of the methods disclosed herein may be conjugated to a solid support suitable for a diagnostic assay (e.g., beads, plates, slides or wells formed from materials such as latex or polystyrene) in accordance with known techniques, such as precipitation. Antibodies or other RBM6-CSF1R fusion polypeptide- or truncated active CSF1R kinase polypeptide-binding reagents may likewise be conjugated to detectable groups such as radiolabels (e.g., ³⁵S, ¹²⁵I, ¹³¹I), enzyme labels (e.g., horseradish peroxidase, alkaline phosphatase), and fluorescent labels (e.g., fluorescein) in accordance with known techniques.

Cell-based assays, such flow cytometry (FC), immuno-histochemistry (IHC), or immunofluorescence (IF) are particularly desirable in practicing the methods of the invention, since such assay formats are clinically-suitable, allow the detection of mutant CSF1R kinase polypeptide expression in vivo, and avoid the risk of artifact changes in activity resulting from manipulating cells obtained from, e.g. a tumor sample in order to obtain extracts. Accordingly, in some preferred embodiment, the methods of the invention are implemented in a flow-cytometry (FC), immuno-histochemistry (IHC), or immunofluorescence (IF) assay format.

Flow cytometry (FC) may be employed to determine the expression of mutant CSF1R kinase polypeptide in a mammalian tumor before, during, and after treatment with a drug targeted at inhibiting CSF1R kinase activity. For example, tumor cells from a bone marrow sample may be analyzed by flow cytometry for RBM6-CSF1R fusion polypeptide expression and/or activation, as well as for markers identifying cancer cell types, etc., if so desired. Flow cytometry may be carried out according to standard methods. See, e.g. Chow et al., Cytometry (Communications in Clinical Cytometry) 46: 72-78 (2001). Briefly and by way of example, the following protocol for cytometric analysis may be employed: fixation of the cells with 2% paraformaldehyde for 10 minutes at 37° C. followed by permeabilization in 90% methanol for 30 minutes on ice. Cells may then be stained with the primary RBM6-CSF1R fusion polypeptide-specific antibody, washed and labeled with a fluorescent-labeled secondary antibody. The cells would then be analyzed on a flow cytometer (e.g. a Beckman Coulter FC500) according to the specific protocols of the instrument used. Such an analysis would identify the level of expressed RBM6-CSF1R fusion polypeptide in the tumor. Similar analysis after treatment of the tumor with a CSF1R-inhibiting therapeutic would reveal the responsiveness of a RBM6-CSF1R fusion polypeptide-expressing tumor to the targeted inhibitor of CSF1R kinase.

Immunohistochemical (IHC) staining may be also employed to determine the expression and/or activation status of mutant CSF1R kinase polypeptide in a mammalian cancer (e.g. AML) before, during, and after treatment with a drug targeted at inhibiting CSF1R kinase activity. IHC may be carried out according to well-known techniques. See, e.g., ANTIBODIES: A LABORATORY MANUAL, Chapter 10, Harlow & Lane Eds., Cold Spring Harbor Laboratory (1988). Briefly, and by way of example, paraffin-embedded tissue (e.g. tumor tissue from a biopsy) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary anti-RBM6-CSF1R fusion polypeptide antibody and secondary antibody; and finally detecting using ABC avidin/biotin method according to manufacturer's instructions.

Immunofluorescence (IF) assays may be also employed to determine the expression and/or activation status of mutant CSF1R kinase polypeptide in a mammalian cancer before, during, and after treatment with a drug targeted at inhibiting CSF1R kinase activity. IF may be carried out according to well-known techniques. See, e.g., J. M. polak and S. Van Noorden (1997) INTRODUCTION TO IMMUNOCYTOCHEMISTRY, 2nd Ed.; ROYAL MICROSCOPY SOCIETY MICROSCOPY HANDBOOK 37, BioScientific/Springer-Verlag. Briefly, and by way of example, patient samples may be fixed in paraformaldehyde followed by methanol, blocked with a blocking solution such as horse serum, incubated with the primary antibody against RBM6-CSF1R fusion polypeptide followed by a secondary antibody labeled with a fluorescent dye such as Alexa 488 and analyzed with an epifluorescent microscope.

Antibodies employed in the above-described assays may be advantageously conjugated to fluorescent dyes (e.g. Alexa488, PE), or other labels, such as quantum dots, for use in multi-parametric analyses along with other signal transduction (EGFR, phospho-AKT, phospho-Erk 1/2) and/or cell marker (cytokeratin) antibodies.

A variety of other protocols, including enzyme-linked immunosorbent assay (ELISA), radio-immunoassay (RIA), and fluorescent-activated cell sorting (FACS), for measuring mutant CSF1R kinase polypeptide are known in the art and provide a basis for diagnosing altered or abnormal levels of RBM6-CSF1R fusion polypeptide expression. Normal or standard values for RBM6-CSF1R fusion polypeptide expression are established by combining body fluids or cell extracts taken from normal mammalian subjects, preferably human, with antibody to RBM6-CSF1R fusion polypeptide under conditions suitable for complex formation. The amount of standard complex formation may be quantified by various methods, but preferably by photometric means. Quantities of RBM6-CSF1R fusion polypeptide expressed in subject, control, and disease samples from biopsied tissues are compared with the standard values. Deviation between standard and subject values establishes the parameters for diagnosing disease.

### Peptide & Nucleic Acid Assays.

Similarly, AQUA peptides for the detection/quantification of expressed mutant CSF1R kinase polypeptide in a biological sample comprising cells from a tumor may be prepared and used in standard AQUA assays, as described in detail in Section E above. Accordingly, in some preferred embodiments of the methods of the invention, the RBM6-CSF1R fusion polypeptide-specific reagent comprises a heavy isotope labeled phosphopeptide (AQUA peptide) corresponding to a peptide sequence comprising the fusion junction of RBM6-CSF1R fusion polypeptide, as described above in Section E.

Mutant CSF1R polypeptide-specific reagents useful in practicing the methods of the invention may also be mRNA, oligonucleotide or DNA probes that can directly hybridize to, and detect, fusion or truncated polypeptide expression transcripts in a biological sample. Such probes are discussed in detail in Section B above. Briefly, and by way of example, formalin-fixed, paraffin-embedded patient samples may be probed with a fluorescein-labeled RNA probe followed by washes with formamide, SSC and PBS and analysis with a fluorescent microscope.

Polynucleotides encoding mutant CSF1R kinase polypeptide may also be used for diagnostic purposes. The polynucleotides which may be used include oligonucleotide sequences, antisense RNA and DNA molecules, and PNAs. The polynucleotides may be used to detect and quantitate gene expression in biopsied tissues in which expression of RBM6-CSF1R fusion polypeptide or truncated active CSF1R kinase polypeptide may be correlated with disease. For example, the diagnostic assay may be used to distinguish between absence, presence, and excess expression of RBM6-CSF1R fusion polypeptide, and to monitor regulation of RBM6-CSF1R fusion polypeptide levels during therapeutic intervention.

In one preferred embodiment, hybridization with PCR probes which are capable of detecting polynucleotide sequences, including genomic sequences, encoding RBM6-CSF1R fusion polypeptide or truncated active CSF1R kinase polypeptide, or closely related molecules, may be used to identify nucleic acid sequences which encode mutant CSF1R polypeptide. The construction and use of such probes is described in Section B above. The specificity of the probe, whether it is made from a highly specific region, e.g., 10 unique nucleotides in the fusion junction, or a less specific region, e.g., the 3′ coding region, and the stringency of the hybridization or amplification (maximal, high, intermediate, or low) will determine whether the probe identifies only naturally occurring sequences encoding mutant CSF1R polypeptide, alleles, or related sequences.

Probes may also be used for the detection of related sequences, and should preferably contain at least 50% of the nucleotides from any of the mutant CSF1R polypeptide encoding sequences. The hybridization probes of the subject invention may be DNA or RNA and derived from the nucleotide sequence of SEQ ID NO: 2, most preferably encompassing the fusion junction (see FIG. 7, bottom panel), or from genomic sequence including promoter, enhancer elements, and introns of the naturally occurring RBM6 and CSF1R polypeptides, as further described in Section B above.

A RBM6-CSF1R fusion polynucleotide or truncated CSF1R polynucleotide of the invention may be used in Southern or northern analysis, dot blot, or other membrane-based technologies; in PCR technologies; or in dip stick, pin, ELISA or chip assays utilizing fluids or tissues from patient biopsies to detect altered CSF1R polypeptide expression. Such qualitative or quantitative methods are well known in the art. In a particular aspect, the nucleotide sequences encoding a mutant CSF1R polypeptide of the invention may be useful in assays that detect activation or induction of various cancers, including leukemias. Mutant CSF1R polynucleotides may be labeled by standard methods, and added to a fluid or tissue sample from a patient under conditions suitable for the formation of hybridization complexes. After a suitable incubation period, the sample is washed and the signal is quantitated and compared with a standard value. If the amount of signal in the biopsied or extracted sample is significantly altered from that of a comparable control sample, the nucleotide sequences have hybridized with nucleotide sequences in the sample, and the presence of altered levels of nucleotide sequences encoding RBM6-CSF1R fusion polypeptide or truncated active CSF1R kinase polypeptide in the sample indicates the presence of the associated disease. Such assays may also be used to evaluate the efficacy of a particular therapeutic treatment regimen in animal studies, in clinical trials, or in monitoring the treatment of an individual patient.

In order to provide a basis for the diagnosis of disease characterized by expression of mutant CSF1R kinase polypeptide, a normal or standard profile for expression is established. This may be accomplished by combining body fluids or cell extracts taken from normal subjects, either animal or human, with a sequence, or a fragment thereof, which encodes RBM6-CSF1R fusion polypeptide or truncated active CSF1R kinase polypeptide, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject values is used to establish the presence of disease.

Once disease is established and a treatment protocol is initiated, hybridization assays may be repeated on a regular basis to evaluate whether the level of expression in the patient begins to approximate that which is observed in the normal patient. The results obtained from successive assays may be used to show the efficacy of treatment over a period ranging from several days to months.

Additional diagnostic uses for mutant CSF1R polynucleotides of the invention may involve the use of polymerase chain reaction (PCR), a preferred assay format that is standard to those of skill in the art. See, e.g., MOLECULAR CLONING, A LABORATORY MANUAL, 2nd. edition, Sambrook, J., Fritsch, E. F. and Maniatis, T., eds., Cold Spring Harbor Laboratory Press, Cold Spring Harbor, N.Y. (1989). PCR oligomers may be chemically synthesized, generated enzymatically, or produced from a recombinant source. Oligomers will preferably consist of two nucleotide sequences, one with sense orientation (5′ to 3′) and another with antisense (3′ to 5′), employed under optimized conditions for identification of a specific gene or condition. The same two oligomers, nested sets of oligomers, or even a degenerate pool of oligomers may be employed under less stringent conditions for detection and/or quantitation of closely related DNA or RNA sequences.

Methods which may also be used to quantitate the expression of RBM6-CSF1R fusion polypeptide or truncated active CSF1R kinase polypeptide include radiolabeling or biotinylating nucleotides, coamplification of a control nucleic acid, and standard curves onto which the experimental results are interpolated (Melby et al., J. Immunol. Methods, 159: 235-244 (1993); Duplaa et al. Anal. Biochem. 229-236 (1993)). The speed of quantitation of multiple samples may be accelerated by running the assay in an ELISA format where the oligomer of interest is presented in various dilutions and a spectrophotometric or colorimetric response gives rapid quantitation.

In another embodiment of the invention, the mutant CSF1R polynucleotides of the invention, as well the adjacent genomic region proximal and distal to them, may be used to generate hybridization probes that are useful for mapping the naturally occurring genomic sequence. The sequences may be mapped to a particular chromosome or to a specific region of the chromosome using well known techniques. Such techniques include fluorescence in-situ hybridization (FISH), FACS, or artificial chromosome constructions, such as yeast artificial chromosomes, bacterial artificial chromosomes, bacterial P1 constructions or single chromosome cDNA libraries, as reviewed in Price, C. M., Blood Rev. 7: 127-134 (1993), and Trask, B. J., Trends Genet. 7: 149-154 (1991).

In one preferred embodiment, FISH is employed (as described in Verma et al. HUMAN CHROMOSOMES: A MANUAL OF BASIC TECHNIQUES, Pergamon Press, New York, N.Y. (1988)) and may be correlated with other physical chromosome mapping techniques and genetic map data. Examples of genetic map data can be found in the 1994 Genome Issue of Science (265: 1981f). Correlation between the location of the gene encoding RBM6-CSF1R fusion polypeptide or truncated active CSF1R kinase polypeptide on a physical chromosomal map and a specific disease, or predisposition to a specific disease, may help delimit the region of DNA associated with that genetic disease. The nucleotide sequences of the subject invention may be used to detect differences in gene sequences between normal, carrier, or affected individuals.

In situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques. Once the disease or syndrome has been crudely localized by genetic linkage to a particular genomic region, for example, AT to 11q22-23 (Gatti et al., Nature 336: 577-580 (1988)), any sequences mapping to that area may represent associated or regulatory genes for further investigation. The nucleotide sequence of the subject invention may also be used to detect differences in the chromosomal location due to translocation, inversion, etc., among normal, carrier, or affected individuals.

Other suitable methods for nucleic acid detection, such as minor groove-binding conjugated oligonucleotide probes (see, e.g. U.S. Pat. No. 6,951,930, “Hybridization-Triggered Fluorescent Detection of Nucleic Acids”) are known to those of skill in the art.

### Biological Samples.

Biological samples useful in the practice of the methods of the invention may be obtained from any mammal in which a cancer characterized by the expression of a RBM6-CSF1R fusion polypeptide or truncated active CSF1R kinase polypeptide is present or developing. In one embodiment, the mammal is a human, and the human may be a candidate for a CSF1R-inhibiting therapeutic for the treatment of a leukemia, e.g. AML. The human candidate may be a patient currently being treated with, or considered for treatment with, a CSF1R kinase inhibitor, such as Gleevec®. In another embodiment, the mammal is large animal, such as a horse or cow, while in other embodiments, the mammal is a small animal, such as a dog or cat, all of which are known to develop cancers, including leukemias.

Any biological sample comprising cells (or extracts of cells) from a mammalian cancer is suitable for use in the methods of the invention. Serum and bone marrow samples may be particularly preferred for patients with leukemia, and may be obtained by standard methods. Circulating tumor cells may also be obtained from serum using tumor markers, cytokeratin protein markers or other methods of negative selection as described (see Ma et al., Anticancer Res. 23(1A): 49-62 (2003)). For cancers involving solid tumors, the biological sample may comprise cells obtained from a tumor biopsy, which maybe be obtained according to standard clinical techniques. For example, aberrant expression of CSF1R has been observed in a spectrum of cancers including breast and ovarian cancer, and its expression stimulates tumor invasion. See, e.g., Kascinski, B., Cancer Treat Res. 107: 285-92 (2002).

A biological sample may comprise cells (or cell extracts) from a cancer in which RBM6-CSF1R fusion polypeptide or truncated active CSF1R kinase polypeptide is expressed and/or activated but wild type CSF1R kinase is not. Alternatively, the sample may comprise cells from a cancer in which both the mutant CSF1R polypeptide and wild type CSF1R kinase are expressed and/or activated, or in which wild type CSF1R kinase and/or RBM-6 are expressed and/or active, but mutant CSF1R polypeptide is not.

Cellular extracts of the foregoing biological samples may be prepared, either crude or partially (or entirely) purified, in accordance with standard techniques, and used in the methods of the invention. Alternatively, biological samples comprising whole cells may be utilized in preferred assay formats such as immunohistochemistry (IHC), flow cytometry (FC), and immunofluorescence (IF), as further described above. Such whole-cell assays are advantageous in that they minimize manipulation of the tumor cell sample and thus reduce the risks of altering the in vivo signaling/activation state of the cells and/or introducing artifact signals. Whole cell assays are also advantageous because they characterize expression and signaling only in tumor cells, rather than a mixture of tumor and normal cells.

In practicing the disclosed method for determining whether a compound inhibits progression of a tumor characterized by a RBM6-CSF1R translocation and/or fusion polypeptide, or a truncated CSF1R kinase polynucleotide and/or or truncated active CSF1R kinase polypeptide, biological samples comprising cells from mammalian bone marrow transplant models or xenografts may also be advantageously employed. Preferred xenografts (or transplant recipients) are small mammals, such as mice, harboring human tumors (or leukemias) that express a mutant CSF1R kinase polypeptide. Xenografts harboring human tumors are well known in the art (see Kal, Cancer Treat Res. 72: 155-69 (1995)) and the production of mammalian xenografts harboring human tumors is well described (see Winograd et al., In Vivo. 1(1): 1-13 (1987)). Similarly the generation and use of bone marrow transplant models is well described (see, e.g., Schwaller, et al., EMBO J. 17: 5321-333 (1998); Kelly et al., Blood 99: 310-318 (2002)). By “cancer characterized by” a RBM6-CSF1R translocation and/or fusion polypeptide, or a truncated CSF1R kinase polynucleotide and/or or truncated active CSF1R kinase polypeptide, is meant a cancer in which such mutant CSF1R gene and/or expressed polypeptide are present, as compared to a cancer in which such translocation, truncated gene, and/or fusion polypeptide are not present.

In assessing mutant CSF1R polynucleotide presence or polypeptide expression in a biological sample comprising cells from a mammalian cancer tumor, a control sample representing a cell in which such translocation and/or fusion protein do not occur may desirably be employed for comparative purposes. Ideally, the control sample comprises cells from a subset of the particular cancer (e.g. leukemia) that is representative of the subset in which the mutation (e.g. RBM6-CSF1R translocation) does not occur and/or the fusion polypeptide is not expressed. Comparing the level in the control sample versus the test biological sample thus identifies whether the mutant CSF1R polynucleotide and/or polypeptide is/are present. Alternatively, since RBM6-CSF1R fusion polynucleotide and/or polypeptide, or truncated CSF1R polynucleotide and/or polypeptide, may not be present in the majority of cancers, any tissue that similarly does not express such mutant CSF1R polypeptide (or harbor the mutant polynucleotide) may be employed as a control.

The methods described below will have valuable diagnostic utility for cancers characterized by mutant CSF1R polynucleotide and/or polypeptide, and treatment decisions pertaining to the same. For example, biological samples may be obtained from a subject that has not been previously diagnosed as having a cancer characterized by a RBM6-CSF1R translocation and/or fusion polypeptide, nor has yet undergone treatment for such cancer, and the method is employed to diagnostically identify a tumor in such subject as belonging to a subset of tumors (e.g. leukemias) in which RBM6-CSF1R fusion polynucleotide and/or polypeptide is present and/or expressed. The methods of the invention may also be employed to monitor the progression or inhibition of a mutant CSF1R kinase polypeptide-expressing cancer following treatment of a subject with a composition comprising a CSF1R kinase-inhibiting therapeutic or combination of therapeutics.

Such diagnostic assay may be carried out subsequent to or prior to preliminary evaluation or surgical surveillance procedures. The identification method of the invention may be advantageously employed as a diagnostic to identify patients having cancer, such as AML, driven by the RBM6-CSF1R fusion protein or by truncated active CSF1R kinase, which patients would be most likely to respond to therapeutics targeted at inhibiting CSF1R kinase activity, such as Gleevec® or its analogues. The ability to select such patients would also be useful in the clinical evaluation of efficacy of future CSF1R-targeted therapeutics as well as in the future prescription of such drugs to patients.

### Diagnostics.

The ability to selectively identify cancers in which a RBM6-CSF1R translocation and/or fusion polypeptide, or a truncated CSF1R polynucleotide or truncated active CSF1R polypeptide, is/are present enables important new methods for accurately identifying such tumors for diagnostic purposes, as well as obtaining information useful in determining whether such a tumor is likely to respond to a CSF1R-inhibiting therapeutic composition, or likely to be partially or wholly non-responsive to an inhibitor targeting a different kinase when administered as a single agent for the treatment of the cancer.

Accordingly, in one embodiment, the invention provides a method for detecting the presence of a mutant CSF1R polynucleotide and/or polypeptide in a cancer, the method comprising the steps of:

(a) obtaining a biological sample from a patient having or at risk of cancer; and

(b) utilizing at least one reagent that detects a mutant CSF1R polynucleotide or polypeptide of the invention to determine whether a mutant CSF1R polynucleotide, RBM6-CSF1R fusion polypeptide, and/or truncated active CSF1R polypeptide is/are present in the biological sample.

In one embodiment, the mutant CSF1R polynucleotide comprises a translocation polynucleotide, and in a preferred embodiment, the translocation polynucleotide comprises a RBM6-CSF1R fusion polynucleotide. In other preferred embodiments, the cancer is a leukemia, such as acute myelogenous leukemia (AML). In still other preferred embodiments, the presence of a mutant CSF1R polypeptide identifies a cancer that is likely to respond to a composition comprising at least one CSF1R kinase-inhibiting therapeutic. Exemplary CSF1R-inhibiting therapeutics include, but are not limited to, Imatinib mesylate (STI-571; Gleevec®) or its analogues, such as SU11248 and GW2580.

In some preferred embodiments, the diagnostic methods of the invention are implemented in a flow-cytometry (FC), immuno-histochemistry (IHC), or immuno-fluorescence (IF) assay format, as described above. In another preferred embodiment, the activity of the RBM6-CSF1R fusion polypeptide and/or truncated active CSF1R kinase polypeptide is detected. In other preferred embodiments, the diagnostic methods of the invention are implemented in a fluorescence in situ hybridization (FISH) or polymerase chain reaction (PCR) assay format, as described above.

The invention further provides a method for determining whether a compound inhibits the progression of a cancer characterized by a RBM6-CSF1R fusion polynucleotide, a truncated CSF1R polynucleotide, a RBM6-CSF1R fusion polypeptide, and/or or a truncated active CSF1R polypeptide, said method comprising the step of determining whether said compound inhibits the expression and/or activity of said RBM6-CSF1R fusion polypeptide or a truncated active CSF1R polypeptide in said cancer. In one preferred embodiment, inhibition of expression and/or activity of the RBM6-CSF1R fusion polypeptide or said truncated active CSF1R polypeptide is determined using at least one reagent that detects an RBM6-CSF1R fusion polynucleotide or polypeptide of the invention. Compounds suitable for inhibition of CSF1R kinase activity are discussed in more detail in Section G below.

Mutant CSF1R polynucleotide probes and polypeptide-specific reagents useful in the practice of the methods of the invention are described in further detail in sections B and D above. In one preferred embodiment, the RBM6-CSF1R fusion polypeptide-specific reagent comprises a fusion polypeptide-specific antibody. In another preferred embodiment, the fusion polypeptide-specific reagent comprises a heavy-isotope labeled phosphopeptide (AQUA peptide) corresponding to the fusion junction of RBM6-CSF1R fusion polypeptide (see FIG. 9 (bottom panel)).

The methods of the invention described above may also optionally comprise the step of determining the level of expression or activation of other kinases, such as wild type CSF1R and EGFR, or other downstream signaling molecules in said biological sample. Profiling both RBM6-CSF1R fusion polypeptide, or truncated active CSF1R kinase polypeptide, expression/activation and expression/activation of other kinases and pathways in a given biological sample can provide valuable information on which kinase(s) and pathway(s) is/are driving the disease, and which therapeutic regime is therefore likely to be of most benefit.

### Compound Screening.

The discovery of the novel RBM6-CSF1R fusion polypeptide and truncated active CSF1R kinase polypeptide described herein also enables the development of new compounds that inhibit the activity of these mutant CSF1R proteins, particularly their CSF1R kinase activity. Accordingly, the invention also provides, in part, a method for determining whether a compound inhibits the progression of a cancer characterized by a RBM6-CSF1R fusion polynucleotide, a truncated CSF1R polynucleotide, a RBM6-CSF1R fusion polypeptide, and/or a truncated active CSF1R kinase polypeptide, said method comprising the step of determining whether said compound inhibits the expression and/or activity of said RBM6-CSF1R fusion polypeptide or said truncated active CSF1R kinase polypeptide in said cancer. In one preferred embodiment, inhibition of expression and/or activity of the RBM6-CSF1R fusion polypeptide or truncated active CSF1R kinase polypeptide is determined using at least one reagent that detects a mutant CSF1R polynucleotide and/or mutant CSF1R polypeptide of the invention. Preferred reagents of the invention have been described above. Compounds suitable for the inhibition of CSF1R kinase activity are described in more detail in Section G below.

The compound may, for example, be a kinase inhibitor, such as a small molecule or antibody inhibitor. It may be a pan-kinase inhibitor with activity against several different kinases, or a kinase-specific inhibitor. CSF1R kinase-inhibiting compounds are discussed in further detail in Section G below. Patient biological samples may be taken before and after treatment with the inhibitor and then analyzed, using methods described above, for the biological effect of the inhibitor on CSF1R kinase activity, including the phosphorylation of downstream substrate protein. Such a pharmacodynamic assay may be useful in determining the biologically active dose of the drug that may be preferable to a maximal tolerable dose. Such information would also be useful in submissions for drug approval by demonstrating the mechanism of drug action. Identifying compounds with such desired inhibitory characteristics is further described in Section G below.

### G. Therapeutic Inhibition of Cancers.

In accordance with the present invention, it has now been shown that the progression of a mammalian cancer (AML) in which RBM6-CSF1R fusion protein or truncated active CSF1R kinase is expressed may be inhibited, in vivo, by inhibiting the activity of CSF1R kinase in such cancer. CSF1R activity in cancers characterized by expression of a mutant CSF1R polypeptide may be inhibited by contacting the cancer (e.g. a tumor) with a CSF1R kinase-inhibiting therapeutic, such as a small-molecule kinase inhibitor like Imatinib mesylate (STI-571; Gleevec®). As further described in Example 3 below, growth inhibition of RBM6-CSF1R fusion protein-expressing leukemia tumors, for example, can be accomplished by inhibiting this fusion kinase using an exemplary CSF1R-inhibiting therapeutic, Gleevec®, or by exemplary siRNA silencing. Accordingly, the invention provides, in part, a method for inhibiting the progression of a cancer that expresses RBM6-CSF1R fusion polypeptide and/or a truncated active CSF1R kinase polypeptide by inhibiting the expression and/or activity of the mutant CSF1R kinase(s) in the cancer.

A CSF1R kinase-inhibiting therapeutic may be any composition comprising at least one compound, biological or chemical, which inhibits, directly or indirectly, the expression and/or activity of CSF1R kinase in vivo, including the exemplary classes of compounds described below. Such compounds include therapeutics that act directly on CSF1R kinase itself, or on proteins or molecules that modify the activity of CSF1R, or that act indirectly by inhibiting the expression of CSF1R. Such compositions also include compositions comprising only a single CSF1R kinase inhibiting compound, as well as compositions comprising multiple therapeutics (including those against other RTKs), which may also include a non-specific therapeutic agent like a chemotherapeutic agent or general transcription inhibitor.

### Small-Molecule Inhibitors.

In some preferred embodiments, a CSF1R-inhibiting therapeutic useful in the practice of the methods of the invention is a targeted, small molecule inhibitor, such as Gleevec® (STI-571), and its analogues. For example, as presently shown (see Examples 3 and 6), administration of Gleevec® to a transgenic leukemia cell line expressing the RBM6-CSF1R fusion protein, or the truncated active CSF1R kinase (lacking the RBM6 moiety), selectively inhibited the progression of the disease in those cells, but not in control cells that do not express the mutant CSF1R proteins. Gleevec®, which specifically binds to and blocks the ATP-binding site of CSF1R kinase (as well as other kinases) thereby preventing phosphorylation and activation of this enzyme, is commercially available and its properties are well known. As presently disclosed, the IC₅₀ of Imatinib on RBM6-CSF1R fusion protein (IC₅₀=1.42 mM), although higher than what is observed for ABL (IC₅₀=0.25 mM) and C-KIT (IC₅₀=0.1 mM), it is still within the therapeutic dose range. See Dewar et al., Blood 105(8): 3127-32 (2005). Thus, Imatinib is an exemplary small molecule inhibitor that should be considered for patients with a cancer characterized by the RBM6-CSF1R fusion or CSF1R mutant kinase.

Other preferred small-molecule inhibitors of CSF1R include SU11248 and GW2580. These compounds are under clinical investigation and their CSF1R kinase inhibitory properties have been described. See, e.g. Murray et al., Clin Exp. Metastasis 20: 757-766 (2003); Conway, Proc. Natl. Acad. Sci. USA 102(44):16078-83 (2005).

Small molecule targeted inhibitors are a class of molecules that typically inhibit the activity of their target enzyme by specifically, and often irreversibly, binding to the catalytic site of the enzyme, and/or binding to an ATP-binding cleft or other binding site within the enzyme that prevents the enzyme from adopting a conformation necessary for its activity. Small molecule inhibitors may be rationally designed using X-ray crystallographic or computer modeling of CSF1R kinase three-dimensional structure, or may found by high throughput screening of compound libraries for inhibition of CSF1R. Such methods are well known in the art, and have been described. Specificity of CSF1R inhibition may be confirmed, for example, by examining the ability of such compound to inhibit CSF1R activity, but not other kinase activity, in a panel of kinases, and/or by examining the inhibition of CSF1R activity in a biological sample comprising leukemia tumor cells, as described above. Such screening methods are further described below.

### Antibody Inhibitors.

CSF1R kinase-inhibiting therapeutics useful in the methods of the invention may also be targeted antibodies that specifically bind to critical catalytic or binding sites or domains required for CSF1R activity, and inhibit the kinase by blocking access of ligands (e.g. CSF), substrates or secondary molecules to a and/or preventing the enzyme from adopting a conformation necessary for its activity. The production, screening, and therapeutic use of humanized target-specific antibodies has been well-described. See Merluzzi et al., Adv Clin Path. 4(2): 77-85 (2000). Commercial technologies and systems, such as Morphosys, Inc.'s Human Combinatorial Antibody Library (HuCAL®), for the high-throughput generation and screening of humanized target-specific inhibiting antibodies are available.

The production of various anti-receptor kinase targeted antibodies and their use to inhibit activity of the targeted receptor has been described. See, e.g. U.S. Patent Publication No. 20040202655, “Antibodies to IGF-I Receptor for the Treatment of Cancers,” Oct. 14, 2004, Morton et al.; U.S. Patent Publication No. 20040086503, “Human anti-Epidermal Growth Factor Receptor Single-Chain Antibodies,” Apr. 15, 2004, Raisch et al.; U.S. Patent Publication No. 20040033543, “Treatment of Renal Carcinoma Using Antibodies Against the EGFr,” Feb. 19, 2004, Schwab et. al. Standardized methods for producing, and using, receptor tyrosine kinase activity-inhibiting antibodies are known in the art. See, e.g., European Patent No. EP1423428, “Antibodies that Block Receptor Tyrosine Kinase Activation, Methods of Screening for and Uses Thereof,” Jun. 2, 2004, Borges et al.

Phage display approaches may also be employed to generate CSF1R-specific antibody inhibitors, and protocols for bacteriophage library construction and selection of recombinant antibodies are provided in the well-known reference text CURRENT PROTOCOLS IN IMMUNOLOGY, Colligan et al. (Eds.), John Wiley & Sons, Inc. (1992-2000), Chapter 17, Section 17.1. See also U.S. Pat. No. 6,319,690, Nov. 20, 2001, Little et al.; U.S. Pat. No. 6,300,064, Oct. 9, 2001, Knappik et al.; U.S. Pat. No. 5,840,479, Nov. 24, 1998, Little et al.; U.S. Patent Publication No. 20030219839, Nov. 27, 2003, Bowdish et al.

A library of antibody fragments displayed on the surface of bacteriophages may be produced (see, e.g. U.S. Pat. No. 6,300,064, Oct. 9, 2001, Knappik et al.) and screened for binding to a soluble dimeric form of a receptor protein tyrosine kinase (like CSF1R). An antibody fragment that binds to the soluble dimeric form of the RTK used for screening is identified as a candidate molecule for blocking constitutive activation of the target RTK in a cell. See European Patent No. EP1423428, Borges et al., supra.

CSF1R binding targeted antibodies identified in screening of antibody libraries as describe above may then be further screened for their ability to block the activity of CSF1R, both in vitro kinase assay and in vivo in cell lines and/or tumors. CSF1R inhibition may be confirmed, for example, by examining the ability of such antibody therapeutic to inhibit CSF1R kinase activity, but not other kinase activity, in a panel of kinases, and/or by examining the inhibition of CSF1R activity in a biological sample comprising cancer cells, as described above. Methods for screening such compounds for CSF1R kinase inhibition are further described above.

### Indirect Inhibitors.

CSF1R-inhibiting compounds useful in the practice of the disclosed methods may also be compounds that indirectly inhibit CSF1R activity by inhibiting the activity of proteins or molecules other than CSF1R kinase itself. Such inhibiting therapeutics may be targeted inhibitors that modulate the activity of key regulatory kinases that phosphorylate or de-phosphorylate (and hence activate or deactivate) CSF1R itself, or interfere with binding of ligands, such as CSF. As with other receptor tyrosine kinases, CSF1R regulates downstream signaling through a network of adaptor proteins and downstream kinases. As a result, induction of cell growth and survival by CSF1R activity may be inhibited by targeting these interacting or downstream proteins. Drugs currently in development that could be used in this manner include AKT inhibitors (Rx-0201) and mTOR inhibitors (rapamycin and its analogs such as CC1-779, Rapamune and RAD001).

CSF1R kinase activity may also be indirectly inhibited by using a compound that inhibits the binding of an activating molecule, such as the Macrophage Colony Stimulating Factor (CSF) 1 or 2, necessary for CSF1R to adopt its active conformation. For example, the production and use of anti-PDGF antibodies has been described. See U.S. Patent Publication No. 20030219839, “Anti-PDGF Antibodies and Methods for Producing Engineered Antibodies,” Bowdish et al. Inhibition of CSF binding to CSF1R directly down-regulates CSF1R activity.

Indirect inhibitors of CSF1R activity may be rationally designed using X-ray crystallographic or computer modeling of CSF1R three dimensional structure, or may found by high throughput screening of compound libraries for inhibition of key upstream regulatory enzymes and/or necessary binding molecules, which results in inhibition of CSF1R kinase activity. Such approaches are well known in the art, and have been described. CSF1R inhibition by such therapeutics may be confirmed, for example, by examining the ability of the compound to inhibit CSF1R activity, but not other kinase activity, in a panel of kinases, and/or by examining the inhibition of CSF1R activity in a biological sample comprising cancer cells, e.g. AML cells, as described above. Methods for identifying compounds that inhibit a cancer characterized by a RBM6-CSF1R translocation and/or fusion polypeptide, or truncated CSF1R polynucleotide and/or truncated active CSF1R kinase, are further described below.

Anti-Sense and/or Transcription Inhibitors.

CSF1R inhibiting therapeutics may also comprise anti-sense and/or transcription inhibiting compounds that inhibit CSF1R kinase activity by blocking transcription of the gene encoding CSF1R and/or the RBM6-CSF1R fusion gene or truncated CSF1R gene. The inhibition of various receptor kinases, including VEGFR, EGFR, and IGFR, and FGFR, by antisense therapeutics for the treatment of cancer has been described. See, e.g., U.S. Pat. Nos. 6,734,017; 6,710,174, 6,617,162; 6,340,674; 5,783,683; 5,610,288.

Antisense oligonucleotides may be designed, constructed, and employed as therapeutic agents against target genes in accordance with known techniques. See, e.g. Cohen, J., Trends in Pharmacol. Sci. 10(11): 435-437 (1989); Marcus-Sekura, Anal. Biochem. 172: 289-295 (1988); Weintraub, H., Sci. AM. pp. 4046 (1990); Van Der Krol et al., BioTechniques 6(10): 958-976 (1988); Skorski et al., Proc. Natl. Acad. Sci. USA (1994) 91: 4504-4508. Inhibition of human carcinoma growth in vivo using an antisense RNA inhibitor of EGFR has recently been described. See U.S. Patent Publication No. 20040047847, “Inhibition of Human Squamous Cell Carcinoma Growth In vivo by Epidermal Growth Factor Receptor Antisense RNA Transcribed from a Pol III Promoter,” Mar. 11, 2004, He et al. Similarly, a CSF1R-inhibiting therapeutic comprising at least one antisense oligonucleotide against a mammalian CSF1R gene (see FIG. 4 (SEQ ID NO: 6)) or RBM6-CSF1R fusion polynucleotide or truncated CSF1R polynucleotide (see FIG. 2 (SEQ ID NO: 2)) may be prepared according to methods described above. Pharmaceutical compositions comprising CSF1R-inhibiting antisense compounds may be prepared and administered as further described below.

### Small Interfering RNA.

Small interfering RNA molecule (siRNA) compositions, which inhibit translation, and hence activity, of CSF1R through the process of RNA interference, may also be desirably employed in the methods of the invention. RNA interference, and the selective silencing of target protein expression by introduction of exogenous small double-stranded RNA molecules comprising sequence complimentary to mRNA encoding the target protein, has been well described. See, e.g. U.S. Patent Publication No. 20040038921, “Composition and Method for Inhibiting Expression of a Target Gene,” Feb. 26, 2004, Kreutzer et al.; U.S. Patent Publication No. 20020086356, “RNA Sequence-Specific Mediators of RNA Interference,” Jun. 12, 2003, Tuschl et al.; U.S. Patent Publication 20040229266, “RNA Interference Mediating Small RNA Molecules,” Nov. 18, 2004, Tuschl et. al.

For example, as presently shown (see Example 3), siRNA-mediated silencing of expression of the RBM6-CSF1R fusion protein in a human leukemia cell line expressing the fusion protein selectively inhibited the progression of the disease in those cells, but not in control cells that do not express the mutant CSF1R protein.

Double-stranded RNA molecules (dsRNA) have been shown to block gene expression in a highly conserved regulatory mechanism known as RNA interference (RNAi). Briefly, the RNAse III Dicer processes dsRNA into small interfering RNAs (siRNA) of approximately 22 nucleotides, which serve as guide sequences to induce target-specific mRNA cleavage by an RNA-induced silencing complex RISC (see Hammond et al., Nature (2000) 404: 293-296). RNAi involves a catalytic-type reaction whereby new siRNAs are generated through successive cleavage of longer dsRNA. Thus, unlike antisense, RNAi degrades target RNA in a non-stoichiometric manner. When administered to a cell or organism, exogenous dsRNA has been shown to direct the sequence-specific degradation of endogenous messenger RNA (mRNA) through RNAi.

A wide variety of target-specific siRNA products, including vectors and systems for their expression and use in mammalian cells, are now commercially available. See, e.g. Promega, Inc. (promega.com); Dharmacon, Inc. (dharmacon.com). Detailed technical manuals on the design, construction, and use of dsRNA for RNAi are available. See, e.g. Dharmacon's “RNAi Technical Reference & Application Guide”; Promega's “RNAi: A Guide to Gene Silencing.” CSF1R-inhibiting siRNA products are also commercially available, and may be suitably employed in the method of the invention. See, e.g. Dharmacon, Inc., Lafayette, Colo. (Cat Nos. M-003162-03, MU-003162-03, D-003162-07 thru-10 (siGENOME™ SMARTselection and SMARTpool® siRNAs).

It has recently been established that small dsRNA less than 49 nucleotides in length, and preferably 19-25 nucleotides, comprising at least one sequence that is substantially identical to part of a target mRNA sequence, and which dsRNA optimally has at least one overhang of 1-4 nucleotides at an end, are most effective in mediating RNAi in mammals. See U.S. Patent Publication No. 20040038921, Kreutzer et al., supra; U.S. Patent Publication No. 20040229266, Tuschl et al., supra. The construction of such dsRNA, and their use in pharmaceutical preparations to silence expression of a target protein, in vivo, are described in detail in such publications.

If the sequence of the gene to be targeted in a mammal is known, 21-23 nt RNAs, for example, can be produced and tested for their ability to mediate RNAi in a mammalian cell, such as a human or other primate cell. Those 21-23 nt RNA molecules shown to mediate RNAi can be tested, if desired, in an appropriate animal model to further assess their in vivo effectiveness. Target sites that are known, for example target sites determined to be effective target sites based on studies with other nucleic acid molecules, for example ribozymes or antisense, or those targets known to be associated with a disease or condition such as those sites containing mutations or deletions, can be used to design siRNA molecules targeting those sites as well.

Alternatively, the sequences of effective dsRNA can be rationally designed/predicted screening the target mRNA of interest for target sites, for example by using a computer folding algorithm. The target sequence can be parsed in silico into a list of all fragments or subsequences of a particular length, for example 23 nucleotide fragments, using a custom Perl script or commercial sequence analysis programs such as Oligo, MacVector, or the GCG Wisconsin Package.

Various parameters can be used to determine which sites are the most suitable target sites within the target RNA sequence. These parameters include but are not limited to secondary or tertiary RNA structure, the nucleotide base composition of the target sequence, the degree of homology between various regions of the target sequence, or the relative position of the target sequence within the RNA transcript. Based on these determinations, any number of target sites within the RNA transcript can be chosen to screen siRNA molecules for efficacy, for example by using in vitro RNA cleavage assays, cell culture, or animal models. See, e.g., U.S. Patent Publication No. 20030170891, Sep. 11, 2003, McSwiggen J. An algorithm for identifying and selecting RNAi target sites has also recently been described. See U.S. Patent Publication No. 20040236517, “Selection of Target Sites for Antisense Attack of RNA,” Nov. 25, 2004, Drlica et al.

Commonly used gene transfer techniques include calcium phosphate, DEAE-dextran, electroporation and microinjection and viral methods (Graham et al. (1973) Virol. 52: 456; McCutchan et al., (1968), J. Natl. Cancer Inst. 41: 351; Chu et al. (1987), Nucl. Acids Res. 15: 1311; Fraley et al. (1980), J. Biol. Chem. 255:10431; Capecchi (1980), Cell 22: 479). DNA may also be introduced into cells using cationic liposomes (Feigner et al. (1987), Proc. Natl. Acad. Sci. USA 84: 7413). Commercially available cationic lipid formulations include Tfx 50 (Promega) or Lipofectamin 200 (Life Technologies). Alternatively, viral vectors may be employed to deliver dsRNA to a cell and mediate RNAi. See U.S Patent Publication No. 20040023390, “siRNA-mediated Gene Silencing with Viral Vectors,” Feb. 4, 2004, Davidson et al.

Transfection and vector/expression systems for RNAi in mammalian cells are commercially available and have been well described. See, e.g. Dharmacon, Inc., DharmaFECT™ system; Promega, Inc., siSTRIKE™ U6 Hairpin system; see also Gou et al. (2003) FEBS. 548, 113-118; Sui, G. et al. A DNA vector-based RNAi technology to suppress gene expression in mammalian cells (2002) Proc. Natl. Acad. Sci. 99, 5515-5520; Yu et al. (2002) Proc. Natl. Acad. Sci. 99, 6047-6052; Paul, C. et al. (2002) Nature Biotechnology 19, 505-508; McManus et al. (2002) RNA 8, 842-850.

siRNA interference in a mammal using prepared dsRNA molecules may then be effected by administering a pharmaceutical preparation comprising the dsRNA to the mammal. The pharmaceutical composition is administered in a dosage sufficient to inhibit expression of the target gene. dsRNA can typically be administered at a dosage of less than 5 mg dsRNA per kilogram body weight per day, and is sufficient to inhibit or completely suppress expression of the target gene. In general a suitable dose of dsRNA will be in the range of 0.01 to 2.5 milligrams per kilogram body weight of the recipient per day, preferably in the range of 0.1 to 200 micrograms per kilogram body weight per day, more preferably in the range of 0.1 to 100 micrograms per kilogram body weight per day, even more preferably in the range of 1.0 to 50 micrograms per kilogram body weight per day, and most preferably in the range of 1.0 to 25 micrograms per kilogram body weight per day. A pharmaceutical composition comprising the dsRNA is administered once daily, or in multiple sub-doses, for example, using sustained release formulations well known in the art. The preparation and administration of such pharmaceutical compositions may be carried out accordingly to standard techniques, as further described below.

Such dsRNA may then be used to inhibit CSF1R expression and activity in a cancer, by preparing a pharmaceutical preparation comprising a therapeutically-effective amount of such dsRNA, as described above, and administering the preparation to a human subject having a cancer expressing RBM6-CSF1R fusion protein or truncated active CSF1R kinase, for example, via direct injection to the tumor. The similar inhibition of other receptor tyrosine kinases, such as VEGFR and EGFR using siRNA inhibitors has recently been described. See U.S. Patent Publication No. 20040209832, Oct. 21, 2004, McSwiggen et al.; U.S. Patent Publication No. 20030170891, Sep. 11, 2003, McSwiggen; U.S. Patent Publication No. 20040175703, Sep. 9, 2004, Kreutzer et al.

### Therapeutic Compositions; Administration.

CSF1R kinase-inhibiting therapeutic compositions useful in the practice of the methods of the invention may be administered to a mammal by any means known in the art including, but not limited to oral or peritoneal routes, including intravenous, intramuscular, intraperitoneal, subcutaneous, transdermal, airway (aerosol), rectal, vaginal and topical (including buccal and sublingual) administration.

For oral administration, a CSF1R-inhibiting therapeutic will generally be provided in the form of tablets or capsules, as a powder or granules, or as an aqueous solution or suspension. Tablets for oral use may include the active ingredients mixed with pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

Capsules for oral use include hard gelatin capsules in which the active ingredient is mixed with a solid diluent, and soft gelatin capsules wherein the active ingredients is mixed with water or an oil such as peanut oil, liquid paraffin or olive oil. For intramuscular, intraperitoneal, subcutaneous and intravenous use, the pharmaceutical compositions of the invention will generally be provided in sterile aqueous solutions or suspensions, buffered to an appropriate pH and isotonicity. Suitable aqueous vehicles include Ringer's solution and isotonic sodium chloride. The carrier may consists exclusively of an aqueous buffer (“exclusively” means no auxiliary agents or encapsulating substances are present which might affect or mediate uptake of the CSF1R-inhibiting therapeutic). Such substances include, for example, micellar structures, such as liposomes or capsids, as described below. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

CSF1R kinase-inhibiting therapeutic compositions may also include encapsulated formulations to protect the therapeutic (e.g. a dsRNA compound) against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811; PCT publication WO 91/06309; and European patent publication EP-A-43075. An encapsulated formulation may comprise a viral coat protein. The viral coat protein may be derived from or associated with a virus, such as a polyoma virus, or it may be partially or entirely artificial. For example, the coat protein may be a Virus Protein 1 and/or Virus Protein 2 of the polyoma virus, or a derivative thereof.

CSF1R-inhibiting compositions can also comprise a delivery vehicle, including liposomes, for administration to a subject, carriers and diluents and their salts, and/or can be present in pharmaceutically acceptable formulations. For example, methods for the delivery of nucleic acid molecules are described in Akhtar et al., 1992, Trends Cell Bio., 2, 139; DELIVERY STRATEGIES FOR ANTISENSE OLIGONUCLEOTIDE THERAPEUTICS, ed. Akbtar, 1995, Maurer et al., 1999, Mol. Membr. Biol., 16, 129-140; Hofland and Huang, 1999, Handb. Exp. Pharmacol., 137, 165-192; and Lee et al., 2000, ACS Symp. Ser., 752, 184-192. Beigelman et al., U.S. Pat. No. 6,395,713 and Sullivan et al., PCT WO 94/02595 further describe the general methods for delivery of nucleic acid molecules. These protocols can be utilized for the delivery of virtually any nucleic acid molecule.

CSF1R-inhibiting therapeutics can be administered to a mammalian tumor by a variety of methods known to those of skill in the art, including, but not restricted to, encapsulation in liposomes, by iontophoresis, or by incorporation into other vehicles, such as hydrogels, cyclodextrins, biodegradable nanocapsules, and bioadhesive microspheres, or by proteinaceous vectors (O'Hare and Normand, International PCT Publication No. WO 00/53722). Alternatively, the therapeutic/vehicle combination is locally delivered by direct injection or by use of an infusion pump. Direct injection of the composition, whether subcutaneous, intramuscular, or intradermal, can take place using standard needle and syringe methodologies, or by needle-free technologies such as those described in Conry et al., 1999, Clin. Cancer Res., 5, 2330-2337 and Barry et al., International PCT Publication No. WO 99/31262.

Pharmaceutically acceptable formulations of CSF1R kinase-inhibitory therapeutics include salts of the above described compounds, e.g., acid addition salts, for example, salts of hydrochloric, hydrobromic, acetic acid, and benzene sulfonic acid. A pharmacological composition or formulation refers to a composition or formulation in a form suitable for administration, e.g., systemic administration, into a cell or patient, including for example a human. Suitable forms, in part, depend upon the use or the route of entry, for example oral, transdermal, or by injection. Such forms should not prevent the composition or formulation from reaching a target cell. For example, pharmacological compositions injected into the blood stream should be soluble. Other factors are known in the art, and include considerations such as toxicity and forms that prevent the composition or formulation from exerting its effect.

Administration routes that lead to systemic absorption (i.e. systemic absorption or accumulation of drugs in the blood stream followed by distribution throughout the entire body), are desirable and include, without limitation: intravenous, subcutaneous, intraperitoneal, inhalation, oral, intrapulmonary and intramuscular. Each of these administration routes exposes the CSF1R-inhibiting therapeutic to an accessible diseased tissue or tumor. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

By “pharmaceutically acceptable formulation” is meant, a composition or formulation that allows for the effective distribution of the nucleic acid molecules of the instant invention in the physical location most suitable for their desired activity. Nonlimiting examples of agents suitable for formulation with the nucleic acid molecules of the instant invention include: P-glycoprotein inhibitors (such as Pluronic P85), which can enhance entry of drugs into the CNS (Jolliet-Riant and Tillement, 1999, Fundam. Clin. Pharmacol., 13, 16-26); biodegradable polymers, such as poly (DL-lactide-coglycolide) microspheres for sustained release delivery after intracerebral implantation (Emerich et al, 1999, Cell Transplant, 8, 47-58) (Alkermes, Inc. Cambridge, Mass.); and loaded nanoparticles, such as those made of polybutylcyanoacrylate, which can deliver drugs across the blood brain barrier and can alter neuronal uptake mechanisms (Prog Neuro-psychopharmacol Biol Psychiatry, 23, 941-949, 1999). Other non-limiting examples of delivery strategies for the CSF1R-inhibiting compounds useful in the method of the invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058.

Therapeutic compositions comprising surface-modified liposomes containing poly (ethylene glycol) lipids (PEG-modified, or long-circulating liposomes or stealth liposomes) may also be suitably employed in the methods of the invention. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

Therapeutic compositions may include a pharmaceutically effective amount of the desired compounds in a pharmaceutically acceptable carrier or diluent. Acceptable carriers or diluents for therapeutic use are well known in the pharmaceutical art, and are described, for example, in REMINGTON'S PHARMACEUTICAL SCIENCES, Mack Publishing Co. (A. R. Gennaro edit. 1985). For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the negatively charged polymer.

Dosage levels of the order of from about 0.1 mg to about 140 mg per kilogram of body weight per day are useful in the treatment of the above-indicated conditions (about 0.5 mg to about 7 g per patient per day). The amount of active ingredient that can be combined with the carrier materials to produce a single dosage form varies depending upon the host treated and the particular mode of administration. Dosage unit forms generally contain between from about 1 mg to about 500 mg of an active ingredient. It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

A CSF1R-inhibiting therapeutic useful in the practice of the invention may comprise a single compound as described above, or a combination of multiple compounds, whether in the same class of inhibitor (i.e. antibody inhibitor), or in different classes (i.e antibody inhibitors and small-molecule inhibitors). Such combination of compounds may increase the overall therapeutic effect in inhibiting the progression of a fusion protein-expressing cancer. For example, the therapeutic composition may a small molecule inhibitor, such as STI-571 (Gleevec®) alone, or in combination with other Gleevec® analogues (e.g. SU11248 or GW2580) targeting CSF1R activity and/or other small molecule inhibitors. The therapeutic composition may also comprise one or more non-specific chemotherapeutic agent in addition to one or more targeted inhibitors. Such combinations have recently been shown to provide a synergistic tumor killing effect in many cancers. The effectiveness of such combinations in inhibiting CSF1R activity and tumor growth in vivo can be assessed as described below.

### Identification of Mutant CSF1R Kinase-Inhibiting Compounds.

The invention also provides, in part, a method for determining whether a compound inhibits the progression of a cancer characterized by a RBM6-CSF1R translocation and/or fusion polypeptide, or a truncated CSF1R polynucleotide and/or truncated active CSF1R kinase polypeptide, by determining whether the compound inhibits the activity of RBM6-CSF1R kinase fusion polypeptide or truncated active CSF1R kinase polypeptide in the cancer. In some preferred embodiments, inhibition of activity of CSF1R is determined by examining a biological sample comprising cells from bone marrow, blood, or a tumor. In another preferred embodiment, inhibition of activity of CSF1R is determined using at least one mutant CSF1R polynucleotide or polypeptide-specific reagent of the invention.

The tested compound may be any type of therapeutic or composition as described above. Methods for assessing the efficacy of a compound, both in vitro and in vivo, are well established and known in the art. For example, a composition may be tested for ability to inhibit CSF1R in vitro using a cell or cell extract in which CSF1R is activated. A panel of compounds may be employed to test the specificity of the compound for CSF1R (as opposed to other targets, such as EGFR or PDGFR).

Another technique for drug screening which may be used provides for high throughput screening of compounds having suitable binding affinity to a protein of interest, as described in published PCT application WO84/03564. In this method, as applied to mutant CSF1R polypeptides, large numbers of different small test compounds are synthesized on a solid substrate, such as plastic pins or some other surface. The test compounds are reacted with mutant CSF1R polypeptide, or fragments thereof, and washed. Bound mutant polypeptide (e.g. RBM6-CSF1R fusion polypeptide or truncated active CSF1R kinase polypeptide) is then detected by methods well known in the art. Purified mutant CSF1R polypeptide can also be coated directly onto plates for use in the aforementioned drug screening techniques. Alternatively, non-neutralizing antibodies can be used to capture the peptide and immobilize it on a solid support.

A compound found to be an effective inhibitor of CSF1R activity in vitro may then be examined for its ability to inhibit the progression of a cancer expressing RBM6-CSF1R fusion polypeptide and/or truncated active CSF1R kinase polypeptide, in vivo, using, for example, mammalian bone marrow transplants (e.g. mice) harboring human leukemias that are driven by the mutant CSF1R protein(s). In this procedure, bone marrow cells known to be driven by mutant CSF1R kinase are transplanted in the mouse. The growth of the cancerous cells may be monitored. The mouse may then be treated with the drug, and the effect of the drug treatment on cancer phenotype or progression be externally observed. The mouse is then sacrificed and the transplanted bone marrow removed for analysis by, etc., IHC and Western blot. Similarly, mammalian xenografts may be prepared, by standard methods, to examine drug response in solid tumors expressing a mutant CSF1R kinase. In this way, the effects of the drug may be observed in a biological setting most closely resembling a patient. The drug's ability to alter signaling in the cancerous cells or surrounding stromal cells may be determined by analysis with phosphorylation-specific antibodies. The drug's effectiveness in inducing cell death or inhibition of cell proliferation may also be observed by analysis with apoptosis specific markers such as cleaved caspase 3 and cleaved PARP.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred.

The teachings of all references cited above and below are hereby incorporated herein by reference. The following Examples are provided only to further illustrate the invention, and are not intended to limit its scope, except as provided in the claims appended hereto. The present invention encompasses modifications and variations of the methods taught herein which would be obvious to one of ordinary skill in the art.

### Example 1

### Identification of CSF1R Kinase Activity in an AML Cell Line by Global Phosphopeptide Profiling

The global phosphorylation profile of kinase activation in several human AML cell lines, including MKPL-1, were examined using a recently described and powerful technique for the isolation and mass spectrometric characterization of modified peptides from complex mixtures (the “IAP” technique, see Rush et al., supra). The IAP technique was performed using a phosphotyrosine-specific antibody (CELL SIGNALING TECHNOLOGY, INC., Beverly, Mass., 2003/04 Cat. #9411) to isolate, and subsequently characterize, phosphotyrosine-containing peptides from extracts of the AML cell lines.

Specifically, the IAP approach was employed go facilitate the identification of tyrosine kinases responsible for STAT5 phosphorylation in the cell lines. STAT5 is a member of the STAT family of transcription factors. The activated tyrosine kinases typically phosphorylate one or more signal transducer and activator (STAT) of transcription factors, which translocate to the cell nucleus and regulate the expression of genes associated with survival and proliferation. The phosphorylation and activation of STAT family members has been previously been described in a wide range of human leukemias. In addition, animal models have demonstrated the important role of STAT in leukemogenesis. STAT5 has been found to be constitutively tyrosine phosphorylated in about 70% of patients with AML. Activating mutations of FLT3 or KIT can account for up to 35% of patients with STAT5 phosphorylation. However, a significant percentage of patients lacking these mutations maintain phosphorylation of STAT5. Hence, it was hypothesized that the upstream activator of STAT5 in some of these patients is an activated tyrosine kinase, and activation of these kinases was examined.

### Cell Culture.

K562 cells were obtained from American Type Culture Collection (ATCC). MKPL-1, GDM-1, NKM-1, CMK, BaF3, and BaF3/BCR-ABL cell lines were generously provided by Dr. Brian Druker (OHSU). BaF3/FLT3-ITD cells were a kind gift from Dr. Donald Small. BaF3 cells were maintained in RPMI-1640 medium (Invitrogen) with 10% fetal bovine serum (FBS) (Sigma) and 1.0 ng/ml IL-3 (R&D Systems). Other cell lines were grown in RPMI-1640 with 10% FBS. 293T cells were grown in DMEM with 10% fetal calf serum.

### Phosphopeptide Immunoprecipitation.

A total of 2×10⁸ cells were lysed in urea lysis buffer (20 mM HEPES pH 8.0, 9M urea, 1 mM sodium vanadate, 2.5 mM sodium pyrophosphate, 1 mM beta-glycerophosphate) at 1.25×10⁸ cells/ml and sonicated. Sonicated lysates were cleared by centrifugation at 20,000×g, and proteins were reduced and alkylated as described previously (see Rush et al., Nat. Biotechnol. 23(1): 94-101 (2005)). Samples were diluted with 20 mM HEPES pH 8.0 to a final urea concentration of 2M. Trypsin (1 mg/ml in 0.001 M HCl) was added to the clarified lysate at 1:100 v/v. Samples were digested overnight at room temperature.

Following digestion, lysates were acidified to a final concentration of 1% TFA. Peptide purification was carried out using Sep-Pak C₁₈ columns as described previously (see Rush et al., supra.). Following purification, all elutions (8%, 12%, 15%, 18%, 22%, 25%, 30%, 35% and 40% acetonitrile in 0.1% TFA) were combined and lyophilized. Dried peptides were resuspended in 1.4 ml MOPS buffer (50 mM MOPS/NaOH pH 7.2, 10 mM Na₂HPO₄, 50 mM NaCl) and insoluble material removed by centrifugation at 12,000×g for 10 minutes.

The phosphotyrosine monoclonal antibody P-Tyr-100 (Cell Signaling Technology) from ascites fluid was coupled non-covalently to protein G agarose beads (Roche) at 4 mg/ml beads overnight at 4° C. After coupling, antibody-resin was washed twice with PBS and three times with MOPS buffer. Immobilized antibody (40 μl, 160 μg) was added as a 1:1 slurry in MOPS IP buffer to the solubilized peptide fraction, and the mixture was incubated overnight at 4° C. The immobilized antibody beads were washed three times with MOPS buffer and twice with ddH₂O. Peptides were eluted twice from beads by incubation with 40 μl of 0.1% TFA for 20 minutes each, and the fractions were combined.

### Analysis by LC-MS/MS Mass Spectrometry.

Peptides in the IP eluate (40 μl) were concentrated and separated from eluted antibody using Stop and Go extraction tips (StageTips) (see Rappsilber et al., Anal. Chem., 75(3): 663-70 (2003)). Peptides were eluted from the microcolumns with 1 μl of 60% MeCN, 0.1% TFA into 7.6 μl of 0.4% acetic acid/0.005% heptafluorobutyric acid (HFBA). The sample was loaded onto a 10 cm×75 μm PicoFrit capillary column (New Objective) packed with Magic C18 AQ reversed-phase resin (Michrom Bioresources) using a Famos autosampler with an inert sample injection valve (Dionex). The column was developed with a 45-min linear gradient of acetonitrile in 0.4% acetic acid, 0.005% HFBA delivered at 280 nl/min (Ultimate, Dionex).

Tandem mass spectra were collected in a data-dependent manner with an LCQ Deca XP Plus ion trap mass spectrometer (ThermoFinnigan), using a top-four method, a dynamic exclusion repeat count of 1, and a repeat duration of 0.5 min.

### Database Analysis & Assignments.

MS/MS spectra were evaluated using TurboSequest (ThermoFinnigan) (in the Sequest Browser package (v. 27, rev. 12) supplied as part of BioWorks 3.0). Individual MS/MS spectra were extracted from the raw data file using the Sequest Browser program CreateDta, with the following settings: bottom MW, 700; top MW, 4,500; minimum number of ions, 20; minimum TIC, 4×10⁵; and precursor charge state, unspecified. Spectra were extracted from the beginning of the raw data file before sample injection to the end of the eluting gradient. The IonQuest and VuDta programs were not used to further select MS/MS spectra for Sequest analysis. MS/MS spectra were evaluated with the following TurboSequest parameters: peptide mass tolerance, 2.5; fragment ion tolerance, 0.0; maximum number of differential amino acids per modification, 4; mass type parent, average; mass type fragment, average; maximum number of internal cleavage sites, 10; neutral losses of water and ammonia from b and y ions were considered in the correlation analysis. Proteolytic enzyme was specified except for spectra collected from elastase digests.

Searches were done against the NCBI human database released on Aug. 24, 2004 containing 27,175 proteins allowing oxidized methionine (M+16) and phosphorylation (Y+80) as dynamic modifications.

In proteomics research, it is desirable to validate protein identifications based solely on the observation of a single peptide in one experimental result, in order to indicate that the protein is, in fact, present in a sample. This has led to the development of statistical methods for validating peptide assignments, which are not yet universally accepted, and guidelines for the publication of protein and peptide identification results (see Carr et al., Mol. Cell. Proteomics 3: 531-533 (2004)), which were followed in this Example. However, because the immunoaffinity strategy separates phosphorylated peptides from unphosphorylated peptides, observing just one phosphopeptide from a protein is a common result, since many phosphorylated proteins have only one tyrosine-phosphorylated site.

For this reason, it is appropriate to use additional criteria to validate phosphopeptide assignments. Assignments are likely to be correct if any of these additional criteria are met: (i) the same sequence is assigned to co-eluting ions with different charge states, since the MS/MS spectrum changes markedly with charge state; (ii) the site is found in more than one peptide sequence context due to sequence overlaps from incomplete proteolysis or use of proteases other than trypsin; (iii) the site is found in more than one peptide sequence context due to homologous but not identical protein isoforms; (iv) the site is found in more than one peptide sequence context due to homologous but not identical proteins among species; and (v) sites validated by MS/MS analysis of synthetic phosphopeptides corresponding to assigned sequences, since the ion trap mass spectrometer produces highly reproducible MS/MS spectra. The last criterion is routinely employed to confirm novel site assignments of particular interest.

All spectra and all sequence assignments made by Sequest were imported into a relational database. Assigned sequences were accepted or rejected following a conservative, two-step process. In the first step, a subset of high-scoring sequence assignments was selected by filtering for XCorr values of at least 1.5 for a charge state of +1, 2.2 for +2, and 3.3 for +3, allowing a maximum RSp value of 10. Assignments in this subset were rejected if any of the following criteria were satisfied: (i) the spectrum contained at least one major peak (at least 10% as intense as the most intense ion in the spectrum) that could not be mapped to the assigned sequence as an a, b, or y ion, as an ion arising from neutral-loss of water or ammonia from a b or y ion, or as a multiply protonated ion; (ii) the spectrum did not contain a series of b or y ions equivalent to at least six uninterrupted residues; or (iii) the sequence was not observed at least five times in all the studies we have conducted (except for overlapping sequences due to incomplete proteolysis or use of proteases other than trypsin). In the second step, assignments with below-threshold scores were accepted if the low-scoring spectrum showed a high degree of similarity to a high-scoring spectrum collected in another study, which simulates a true reference library-searching strategy. All spectra supporting the final list of assigned sequences (not shown here) were reviewed by at least three scientists to establish their credibility.

The foregoing IAP analysis identified 512 non-redundant phosphotyrosine-containing peptides, 437 phosphotyrosine sites, and 300 tyrosine phosphorylated proteins, the majority of which are novel, from MKPL-1 cells (data not shown). Among tyrosine phosphorylated kinases, several of those detected are not normally detected by MS analysis in other leukemia cell lines (unpublished data), including CSF1R, C-KIT, Jak2, and Jak3.

DNA sequencing analysis was conducted as previously described (see O'Farrell et al., Clin Cancer Res. 9(15): 5465-76 (2003); Goemans et al., Leukemia 19(9): 153642 (2005)) to examine whether any of these kinases contained mutations. This analysis did not find any mutations in the kinase domains of CSF1R, Jak2, and Jak3 (data not shown) in the MKPL-1 cell line. Denaturing-HPLC analysis revealed that this cell line did not contain activating mutations in either FLT3 or KIT.

### Example 2

### Western Blot Analysis of CSF1R Kinase Expression in an AML Cell Line

The observation that the MKPL-1 AML cell line—but not the other AML cell lines—expresses activated CSF1R kinase was confirmed by Western blot analysis of cell extracts using antibodies specific for CSF1R and other receptor tyrosine kinases (RTKs) and downstream kinases.

MKPL-1 cells were lysed in 1× cell lysis buffer (Cell Signaling Technology) supplemented with Protease Arrest™ (G Biosciences) and separated by electrophoresis. All antibodies and reagents for immunoblotting were from Cell Signaling Technology, Inc. (Beverly, Mass.). Western blotting was carried out as described in “Western Immunoblotting Protocol” (Cell Signaling Technology, Inc., 2005-2006 catalogue).

FIG. 5 shows the western blot results. While wild type CSF1R was detected in two of the AML cell lines (GDM-1 and NKM-1), a truncated form of CSF1R was detected in the MKPL-1 cell line (see panel A, left column). In addition, phosphorylation of CSF1R kinase's downstream targets, STAT5 and ERK, was also detected in the MKPL-1 cell line (as well as cell lines CMK and K562), validating the presence of activated (but truncated) CSF1R kinase in this AML cell line (see FIG. 5, panel B). Beta-actin expression was used as a control.

### Example 3

### Growth Inhibition of Truncated CSF1R Kinase-Expressing Mammalian AML Cell Lines using Gleevec® and siRNA

In order to confirm that the truncated form of CSF1R is driving cell growth and survival in the MKPL-1 AML cell line, the ability of both a CSF1R-inhibitor, Gleevec®, and siRNA to inhibit growth of these cells was examined.

### Gleevec® Inhibition.

Imatinib (STI-571; Gleevec® (Novartis, Basel, Switzerland)) is a potent tyrosine kinase inhibitor for ABL, ARG, PDGFR a and b, and C-KIT, and also inhibits CSF1R. To confirm that CSF1R drives the proliferation of MKPL-1 cells, the effect of Imatinib on the growth of MKPL-1 cells was examined. For Western blotting, cells were treated for two hours with 10 μM Imatinib before lysis. For dose response curves, cells were incubated for 48 hours in the presence of Imatinib, and the number of viable cells was determined with the CellTiter 96 AQᵤₑₒᵤₛ One solution cell proliferation assay (Promega). IC₅₀ was calculated with the use of OriginPro 6.1 software (OriginLab). The percentage of apoptotic cells at 48 hours was determined by flow cytometric analysis of Cleaved-Caspase-3 (Cell Signaling Technology).

A standard MTT cell proliferation assay (see Mosmann, J. Immunol. Methods. 65(1-2): 55-63 (1983)) was performed on the MKPL-1 cell line (as well as AML cell line GDM-1) using a range of Gleevec® concentrations. The K562 cell line, which is known to be driven by the BCR/ABL translocation and inhibited by Gleevec®, was employed as a positive control. CMK was employed as a negative control. The results of the assay are presented in FIG. 6 (panel B). The results confirm that the MKPL-1 cell line (as well as the GDM-1 cell line, which expresses wild type CSF1R) is inhibited by Gleevec®, while the control cells are not. MKPL-1 cells were inhibited by Imatinib, though at much higher concentrations than required in K562 cells expressing BCR-ABL. The concentration of Imatinib required to inhibit MKPL-1 cells by 50 percent (IC₅₀) was 1.26 mM, where as the IC₅₀ for BCR-ABL in K562 cells was 0.31 mM.

Furthermore, treatment of MKPL-1 cells with Imatinib resulted in induction of apoptosis (% cleaved caspase-3), which was not observed in the control cell line CMK, a known AML-M7 cell line (see FIG. 6, panel C). Wild type CSF1R has been reported as a target of Imatinib, with an IC₅₀ of 1.42 mM, which is very similar to what we observed in MKPL-1 cells expressing the truncated form of the kinase disclosed herein. These data indicate that truncated CSF1R is the target of Imatinib in MKPL-1 cells.

To further confirm the effect of Gleevec® on the MKPL-1 AML cell line, Western blot analysis was performed on several of the cell lines following exposure to a range of Gleevec® concentrations, and the activity of both CSF1R (see FIG. 5 (panel b) and its downstream target, STAT5 (see FIG. 6 (panel A), examined. The results indicate that phosphorylation of the truncated form of CSF1R in MKPL-1 cells is inhibited by Imatinib. In addition, phosphorylation of its downstream target STAT5 and ERK were inhibited as well (see FIG. 6 (panel a)). These findings indicate that a truncated form of CSF1R is constitutively activated in MKPL-1, and is sensitive to inhibition with a small molecule inhibitor.

siRNA Inhibition.

To further examine whether CSF1R contributes to the growth and viability of the AML (MKPL-1) cells, the expression of CSF1R was downregulated with siRNA. For Western blotting, cells were treated for two hours with 10 μM Imatinib before lysis. For dose response curves, cells were incubated for 48 hours in the presence of Imatinib, and the number of viable cells was determined with the CellTiter 96 AQᵤₑₒᵤₛ One solution cell proliferation assay (Promega). IC₅₀ was calculated with the use of OriginPro 6.1 software (OriginLab). The percentage of apoptotic cells at 48 hours was determined by flow cytometric analysis of Cleaved-Caspase-3 (Cell Signaling Technology).

CSF1R SMARTpool siRNA duplexes (proprietary target sequences—data not shown) were purchased from Dharmacon Research, Inc. (Lafayette, Colo.). A non-specific SMARTpool siRNA was used as a control. Cells were transfected with the siRNA via electroporation. Briefly, 2×10⁷ cells were pulsed once (MKPL-1 20 ms; 275V, K562 20 ms; 285V) using a square-wave electroporator (BTX Genetronics, San Diego, Calif.), incubated at room temperature for 30 minutes and transferred to T150 flasks with 30 ml RPMI-1640/10% FBS.

As shown in FIG. 7, immunoblot analysis revealed that the expression of CSF1R was specifically and significantly reduced at 72 hours following transfection of the siRNA into MKPL-1 cells. This is accompanied by a decrease in the phosphorylation of STAT5 and ERK (FIG. 7, panel A). As expected, down regulation of CSF1R resulted in inhibition of cell growth (FIG. 7, panel B). Moreover, treatment with CSF1R siRNA resulted in increased apoptosis of the MKPL-1 cell line (FIG. 7, panel C). These results not only indicate that the mutant/truncated CSF1R kinase in the MKPL-1 cell line is driving the proliferation and growth of these AML cells, but that such growth and proliferation may be inhibited by using siRNA to inhibit CSF1R kinase expression.

### Example 4

### Isolation & Sequencing of RBM6-CSF1R Fusion Gene

Given the presence of the truncated form of CSF1R kinase detected in an AML cell line (MKPL-1), 5′ rapid amplification of cDNA ends on the sequence encoding the kinase domain of CSF1R was conducted in order to determine whether a chimeric CSF1R transcript was present.

### Rapid Amplification of Complementary DNA Ends

RNeasy Mini Kit (Qiagen) was used to extract RNA from human leukemia cell lines. DNA was extracted with the use of DNeasy Tissue Kit (Qiagen). Rapid amplification of cDNA ends was performed with the use of 5′ RACE system (Invitrogen) with primers CSF1R-P1 for cDNA synthesis and CSF1R-P2 and CSF1R-P3 for a nested PCR reaction.

### PCR Assay

For RT-PCR, first-strand cDNA was synthesized from 2.5 mg of total RNA with the use of SuperScrip™ III first-strand synthesis system (Invitrogen) with oligo (dT)₂₀. Then, the RBM6-CSF1R fusion gene was amplified with the use of primer pairs RBM6-F1 and CSF1R-P3. The reciprocal fusion was detected with the use of primer pairs CSF1R-F and RBM6-R. Wild type RBM6 and CSF1R were amplified with the use of primer pairs RBM6-F1 and RBM6-R, CSF1R-F and CSF1R-P3, respectively. For genomic PCR, amplification of the fusion gene was performed with the use of Platinum Taq DNA polymerase high fidelity (Invitrogen) with primer pairs gRBM6-F1 and gCSF1R-R1, or gRBM6-F1 and gCSF1R-R2.

### Constructs

The open reading frame of the RBM6-CSF1R fusion gene was amplified by PCR from cDNA of MKPL-1 cells with the use of Platinum Taq DNA polymerase high fidelity (Invitrogen) and primer pairs RBM6-Fc1 and CSF1R-Rc. This PCR product was cloned in the retroviral vector MSCV-Neo or MSCV-GFP. Construct with deletion mutation was obtained by PCR from RBM6-CSF1R clone with primer pairs RBM6-Fc2 and CSF1R-Rc. The following primers were used:

RBM6-F1:

(SEQ ID NO: 9)

5′GACCTGCTAACAGAACTGGACCTT
RBM6-R:

(SEQ ID NO: 10)

5′CTCTGAAGTCAACAGCGTGAGCAT
CSF1R-F:

(SEQ ID NO: 11)

5′ATCGAGAGCTATGAGGGCAACAGT
CSF1R-P1:

(SEQ ID NO: 12)

5′CCTTCCTTCGCAGAAAGTTGAGCA
CSF1R-P2:

(SEQ ID NO: 13)

5′AAAGTTGAGCAGGTCGCCATAGCA
CSF1R-P3:

(SEQ ID NO: 14)

5′AGCAACAGTACTCCGTGATGACCA
gRBM6-F1:

(SEQ ID NO: 15)

5′TGTAAAAGGAGGGCCAGTTCCTCGTGTTCTGAAATGGGAGCA
gCSF1R-R1:

(SEQ ID NO: 16)

5′AACAGCTATGACCATGCAAACTGCAGGTTGTTCC
gCSF1R-R2:

(SEQ ID NO: 17)

5′AACAGCTATGACCATGATCTTCACAGCCACCTTCAGGACA
GAPDH-F:

(SEQ ID NO: 18)

5′TGGAAATCCCATCACCATCT
GAPDH-R:

(SEQ ID NO: 19)

5′GTCTTCTGGGTGGCAGTGAT
RBM6-Fc1:

(SEQ ID NO: 20)

5′CGGAATTCGATAAAAAGAGATGTGGGGGGAT
RBM6-Fc2:

(SEQ ID NO: 21)

5′CGGAATTCACCATGAAGTGGGAGTTCCCCCGGA
CSF1R-Rc:

(SEQ ID NO: 22)

5′CGGAATTCCGTCAACTCCTCAGCAGAACT

FIG. 8 shows the detection of the PCR amplification product after 2 rounds. Sequence analysis of the resultant product revealed that the split kinase domain of CSF1R was fused to RNA Binding Motif 6 (RBM6) gene N-terminus (see FIG. 1, panel B). The RBM6-CSF1R fusion gene was in-frame and fused the first 36 amino acids of RBM6 to the last 399 amino acids of CSF1R (see FIG. 1, panel B). RBM6 was located on chromosome 3p21.3, whereas CSF1R was on chromosome 5q33. Thus, the fusion gene was created by t(3;5)(p21;q33).

The fusion of RBM6 and CSF1R was confirmed by reverse-transcriptase-PCR on RNA and PCR on DNA from MKPL-1 cells (see FIGS. 9 and 10). The reciprocal CSF1R-RBM6 fusion gene was not detected in RNA. Moreover, the MKPL-1 cell line did not express wild-type CSF1R, but did express wild-type RBM6 (see FIG. 9), strongly suggesting that RBM6-CSF1R fusion is the major genetic abnormality responsible for proliferation and survival the MKPL-1 AML cell line.

### Example 5

### RBM6-CSF1R Fusion Protein Drives Growth and Survival of Transformed Mammalian Cell Line

In order to confirm that expression of the CSF1R-RBM6 fusion protein can transform normal cells into a cancerous phenotype, BaF3 cells were transformed with the cDNA construct described above. BaF3 cells were a kind gift from Dr. Donald Small. Cells were maintained in RPMI-1640 medium (Invitrogen) with 10% fetal bovine serum (FBS) (Sigma) and 1.0 ng/ml IL-3 (R&D Systems).

Production of retroviral supernatant and transduction was carried out as previously described. See Schwaller et al., Embo J. 17(18): 5321-33 (1998). BaF3 cells were transduced with retroviral supernatant containing either the MSCV-Neo/RBM6-CSF1R or MSCV-Neo/CSF1R (truncation) vectors, respectively, and selected for G418 (1 mg/ml). IL-3 independent growth was accessed by plating transduced BaF3 cells in IL-3 free medium, after the cells were washed three times in PBS. For Western blotting, cells were treated for two hours with 10 μM Imatinib before lysis. For dose response curves, cells were incubated for 48 hours in the presence of Imatinib (Gleevec®), and the number of viable cells was determined with the CellTiter 96 AQᵤₑₒᵤₛ One solution cell proliferation assay (Promega). IC₅₀ was calculated with the use of OriginPro 6.1 software (OriginLab). The percentage of apoptotic cells at 48 hours was determined by flow cytometric analysis of Cleaved-Caspase-3 (Cell Signaling Technology).

As expected, the expression of RBM6-CSF1R fusion protein transformed the murine hematopoietic cell line BaF3 to interleukin-3-independent growth (see FIG. 11, panel C) and was constitutively tyrosine-phosphorylated in these cells. Analysis of the phosphorylation status of STAT5, ERK, and AKT indicated that these signaling molecules are downstream targets of RBM6-CSF1R, as expected (see FIG. 11, panel B).

### Example 6

### Truncated CSF1R Kinase Drives Transformed Cell Growth Independent of Presence of RBM6 Moiety

In order to examine whether the RBM6 moiety (amino acids 1-36 of the RBM6-CSF1R fusion protein) is necessary for the kinase function of the truncated CSF1R kinase, a deletion construct was prepared. BaF3 cells were prepared and transformed with the deletion construct substantially as described in Example 5 above.

Surprisingly, deletion of the RBM6 moiety (amino acids 1-36) did not abrogate truncated CSF1R-driven cell growth, indicating that the RBM6 part of the fusion protein is not essential for the activation of the chimeric CSF1R kinase. See FIG. 11, panel C.

### Example 7

### Development of Murine Bone Marrow Transplant Models for Truncated CSF1R Kinase or RBM6-CSF1R Fusion Protein

The in vivo transforming ability of activated CSF1R may be further shown using murine bone marrow transplantation experiments, as previously described. See, e.g., Stover et al., Blood 106(9): 3206-3213 (2005).

Briefly, MSCV-GFP retroviral supernatants were titered by transducing Ba/F3 cells with supernatant (plus polybrene, 10 μg/mL) and analyzing for the percentage of GFP+ cells by flow cytometry at 48 hours after transduction. Balb/C donor mice (Taconic, Germantown, N.Y.) were treated for 5 to 6 days with 5-fluorouracil (150 mg/kg, intraperitoneal injection). Bone marrow cells from donor mice were harvested, treated with red blood cell lysis buffer, and cultured 24 hours in transplantation medium (RPMI+10% FBS+6 ng/mL IL-3, 10 ng/mL IL-6, and 10 ng/mL stem-cell factor). Cells were treated by spin infection with retroviral supernatants (1 mL supernatant per 4×10⁶ cells, plus polybrene) and centrifuged at 1800 g for 90 minutes. The spin infection was repeated 24 hours later, and the cells were then washed, resuspended in Hanks balanced salt solution, and injected into lateral tail veins of lethally irradiated (2×4.5 Gy [450 rad]) Balb/C recipient mice (Taconic) at 0.5 to 1.0×10⁶ cells/mouse

Such analysis would show the transforming properties of activate CSF1R in vivo. Also, it is a useful model for testing small molecular inhibitors against activated CSF1R kinase.

### Example 8

### Detection of RBM6-CSF1R Fusion Protein Expression in a Human Cancer Sample Using FISH Assay

The presence of truncated CSF1R kinase and/or RBM6-CSF1R fusion protein in a human cancer sample may be detected using a fluorescence in situ hybridization (FISH) assay, as previously described. See, e.g., Verma et al. HUMAN CHROMOSOMES: A MANUAL OF BASIC TECHNIQUES, Pergamon Press, New York, N.Y. (1988).

Briefly and by way of example, bone marrow samples may be obtained from a patient having AML using standard techniques. FISH probes against truncated CSF1R kinase or RBM6-CSF1R fusion protein are constructed, and FISH analysis is performed as described below. See, e.g., Dierlamm et al., Genes, Chromosomes and Cancer 6:261-4 (1996). Probes Preparation: At room temperature mix 7 ÅμL of LSI Hybridization Buffer, 1 ÅμL LSI DNA probe, and 2 ÅμL purified H₂O. Centrifuge for 1-3 seconds, vortex and then re-centrifuge. Heat for 5 minutes in a 73° C. water bath, and place on a slide warmer set to 45-50° C. Slide Preparation: Mark hybridization areas with a diamond tipped scribe. Immerse the slide in the 73+/−1° C. denaturant bath (70% formamide/2×SSC) for 5 minutes. If metaphase chromosome morphology is problematic, a denaturant temperature of 70-73° C. may provide for better results. Dehydrate slide 1 minute in 70% EtOH, 1 minute in 85% EtOH, and 1 minute in 100% EtOH. Dry slide and place on a 45-50° C. slide warmer for 2 minutes.

Hybridization: Apply 10 ÅμL of probe mix to slide. Apply coverslip immediately upon placing probe on slide, and seal coverslip with diluted rubber cement. Place slide in a pre-warmed humidified box and allow for hybridization to proceed overnight for 12-16 hrs in a 37° C. incubator. Prepare one wash tank with 0.4×SSC/0.3% NP-40 and place into the 73+/−1 CC water bath for at least 30 minutes. Discard after 1 day of use. Prepare a second tank of 2×SSC/0.1% NP-40 at room temperature. Remove rubber cement seal and the coverslip and immediately place into wash tank (0.4×SSC/0.3% NP-40), agitating the slide for 1-3 seconds. Repeat to a maximum of four slides, then leave all slides in the coplin jar for 2 minutes. Do not remove the coverslips from several slides before placing any of the slides in the wash bath. Begin timing the incubation when the last slide has been added to the wash bath. Wash slide in 2×SSC/0.1% NP-40 at room temperature for 5 seconds-1 minute, agitating for 1-3 seconds as the slide is placed in the bath.

Allow slide to air dry in darkness. Interpretation: Apply 10 ÅμL DAPI II counterstain to the target area of slide and add coverslip. View slide using a suitable filter set. Storage: Store slide at −20° C. in dark.

Such an analysis will identify a patient having a cancer characterized by expression of the truncated CSF1R kinase (and/or RBM6-CSF1R fusion protein), which patient is a candidate for treatment using a CSF1R-inhibiting therapeutic, such as Gleevec®.

### Example 9

### Detection of Mutant CSF1R Kinase Expression in a Human Cancer Sample Using PCR Assay

The presence of truncated CSF1R kinase and/or RBM6-CSF1R fusion protein in a human cancer sample may be detected using either genomic or reverse transcriptase (RT) polymerase chain reaction (PCR), previously described. See, e.g., Cools et al., N. Engl. J. Med. 348:1201-1214 (2003).

Briefly and by way of example, bone marrow samples may be obtained from a patient having AML using standard techniques. PCR probes against truncated CSF1R kinase or RBM6-CSF1R fusion protein are constructed. RNeasy Mini Kit (Qiagen) may be used to extract RNA from human bone marrow samples. DNA may be extracted with the use of DNeasy Tissue Kit (Qiagen). For RT-PCR, first-strand cDNA is synthesized from, e.g., 2.5 μg of total RNA with the use, for example, of SuperScript™ III first-strand synthesis system (Invitrogen) with oligo (dT)₂₀. Then, the RBM6-CSF1R fusion gene is amplified with the use of primer pairs, e.g. RBM6-F1 and CSF1R-P3 (see Example 4 above). For genomic PCR, amplification of the fusion gene may be performed with the use of Platinum Taq DNA polymerase high fidelity (Invitrogen) with primer pairs, e.g. gRBM6-F1 and gCSF1R-R1, or gRBM6-F1 and gCSF1R-R2 (see Example 4, above).

Such an analysis will identify a patient having a cancer characterized by expression of the truncated CSF1R kinase (and/or RBM6-CSF1R fusion protein), which patient is a candidate for treatment using a CSF1R-inhibiting therapeutic, such as Gleevec®.

What is claimed is:

1. An isolated polynucleotide comprising a nucleotide sequence at least 95% identical to a sequence selected from the group consisting of:
(a) a nucleotide sequence encoding a RNA Binding Protein-6-Macrophage Colony Stimulating Factor-1 Receptor (RBM6-CSF1R) fusion polypeptide comprising the amino acid sequence of SEQ ID NO: 1;
(b) a nucleotide sequence encoding a RBM6-CSF1R fusion polypeptide, said nucleotide sequence comprising the nucleotide sequence of SEQ ID NO: 2;
(c) a nucleotide sequence encoding a RBM6-CSF1R fusion polypeptide comprising the N-terminal amino acid sequence of RBM-6 (residues 1-36 of SEQ ID NO: 3) and the split kinase domain of CSF1R (residues 582-910 of SEQ ID NO: 5);
(d) a nucleotide sequence comprising the N-terminal nucleotide sequence of RBM-6 (residues 1-108 of SEQ ID NO: 4) and the split kinase domain nucleotide sequence of CSF1R (residues 1746-2730 of SEQ ID NO: 6);
(e) a nucleotide sequence comprising at least six contiguous nucleotides encompassing the fusion junction (residues 106-111 of SEQ ID NO: 2) of a RBM6-CSF1R fusion polynucleotide;
(f) a nucleotide sequence encoding a polypeptide comprising at least six contiguous amino acids encompassing the fusion junction (residues 36-37 of SEQ ID NO: 1) of a RBM6-CSF1R fusion polypeptide;
(g) a nucleotide sequence encoding a truncated active CSF1R kinase polypeptide comprising residues 574-972 of SEQ ID NO: 5, but not comprising the extracellular or transmembrane domains of wild type CSF1R;
(h) a nucleotide sequence encoding a truncated active CSF1R kinase polypeptide, said nucleotide sequence comprising nucleotides 1722-2916 of SEQ ID NO: 6, but not encoding the extracellular or transmembrane domains of wild type CSF1R;
(i) a nucleotide sequence encoding a truncated active CSF1R kinase polypeptide comprising the split kinase domain of CSF1R (residues 582-910 of SEQ ID NO: 5) but not comprising the extracellular or transmembrane domains of wild type CSF1R;
(j) a nucleotide sequence comprising up to thirty contiguous nucleotides encompassing the truncation point (residue 1722 of SEQ ID NO: 6) of wild type CSF1R kinase polynucleotide;
(k) a nucleotide sequence complementary to any of the nucleotide sequences of (a)-(j).

2. The isolated polynucleotide of claim 1, wherein said nucleotide sequence of (b) comprises the coding nucleotide sequence of the cDNA clone contained in ATCC Deposit No. PTA-7309.

3. The isolated polynucleotide of claim 1, wherein said nucleotide sequence of (h) comprises nucleotides 109-1305 of the coding sequence of the cDNA clone contained in ATCC Deposit No. PTA-7309.

4. An isolated polynucleotide that hybridizes under stringent hybridization conditions to a polynucleotide of claim 1, wherein said isolated polynucleotide that hybridizes does not hybridize under stringent hybridization conditions to a polynucleotide having a nucleotide sequence consisting of only A residues or of only T residues.

5. The isolated polynucleotide of claim 4, wherein said polynucleotide further comprises a detectable label.

6. A method for producing a recombinant vector comprising inserting an isolated nucleic acid molecule of claim 1 into a vector.

7. A recombinant vector produced by the method of claim 6.

8. A method for making a recombinant host cell comprising introducing the recombinant vector of claim 7 into a host cell.

9. A recombinant host cell produced by the method of claim 8.

10. A method for producing a recombinant RBM6-CSF1R fusion polypeptide or truncated active CSF1R polypeptide, said method comprising culturing the recombinant host cell of claim 9 under conditions suitable for the expression of said fusion polypeptide and recovering said polypeptide.

11. An isolated polypeptide comprising an amino acid sequence at least 95% identical to a sequence selected from the group consisting of:
(a) an amino acid sequence encoding a RBM6-CSF1R fusion polypeptide comprising the amino acid sequence of SEQ ID NO: 1;
(b) an amino acid sequence encoding a RBM6-CSF1R fusion polypeptide comprising the N-terminal amino acid sequence of RBM-6 (residues 1-36 of SEQ ID NO: 3) and the split kinase domain of CSF1R (residues 582-910 of SEQ ID NO: 5);
(c) an amino acid sequence encoding a polypeptide comprising at least six contiguous amino acids encompassing the fusion junction (residues 36-37 of SEQ ID NO: 1) of a RBM6-CSF1R fusion polypeptide;
(d) an amino acid sequence encoding a truncated active CSF1R kinase polypeptide comprising the amino acid sequence of residues 574-972 of SEQ ID NO: 5, but not comprising the extracellular or transmembrane domains of wild type CSF1R; and
(e) an amino acid sequence encoding a truncated active CSF1R kinase polypeptide comprising the split kinase domain of CSF1R (residues 582-910 of SEQ ID NO: 5), but not comprising the extracellular or transmembrane domains of wild type CSF1R.

12. The isolated polypeptide of claim 10, wherein said amino acid sequence of (a) comprises the RBM6-CSF1R fusion polypeptide sequence encoded by the cDNA in ATCC Deposit No. PTA-7309.

13. The isolated polypeptide of claim 10, wherein said amino acid sequence of (d) comprises the truncated active CSF1R polypeptide sequence (residues 574-972 of SEQ ID NO: 1) encoded by the cDNA in ATCC Deposit No. PTA-7309.

14. A recombinant RBM6-CSF1R fusion polypeptide or truncated active CSF1R polypeptide produced using the recombinant vector of claim 7 or the recombinant host cell of claim 9.

15. An isolated reagent that specifically binds to or detects a RBM6-CSF1R fusion polypeptide or truncated active CSF1R polypeptide of claim 10, but does not bind to or detect either wild type RBM-6 or wild type CSF1R.

16. The isolated reagent of claim 15, wherein said reagent is an antibody or a heavy-isotope labeled (AQUA) peptide.

17. The heavy isotope labeled (AQUA) peptide of claim 16, wherein said peptide comprises the amino acid sequence of the fusion junction of RBM6-CSF1R fusion polypeptide or truncation point within the juxtamembrane domain of CSF1R.

18. A method for detecting the presence of a mutant CSF1R polynucleotide and/or polypeptide in a cancer, said method comprising the steps of:
(a) obtaining a biological sample from a patient having or at risk of cancer; and
(b) utilizing at least one reagent that detects a polynucleotide of claim 1 and/or at least one reagent of claim 15 to determine whether a mutant CSF1R polynucleotide, RBM6-CSF1R fusion polypeptide, and/or truncated active CSF1R polypeptide is present in said biological sample.

19. The method of claim 18, wherein said mutant CSF1R polynucleotide comprises a translocation polynucleotide.

20. The method of claim 19, wherein said translocation polynucleotide comprises a RBM6-CSF1R fusion polynucleotide.

21. The method of claim 18, wherein said cancer is leukemia.

22. The method of claim 21, wherein said leukemia is acute myelogenous leukemia (AML).

23. The method of claim 18, wherein the presence of a mutant CSF1R polynucleotide or polypeptide identifies a cancer that is likely to respond to a composition comprising at least one CSF1R kinase-inhibiting therapeutic.

24. The method of claim 23, wherein said CSF1R kinase-inhibiting therapeutic is Imatinib mesylate (STI-571) or its analogues.

25. The method of claim 20, wherein said Imatinib mesylate (STI-571) analogue is SU11248 or GW2580.

26. The method of claim 20, wherein the method is implemented in a flow-cytometry (FC), immuno-histochemistry (IHC), or immuno-fluorescence (IF) assay format.

27. The method of claim 20, wherein the method is implemented in a fluorescence in situ hybridization (FISH) or polymerase chain reaction (PCR) assay format.

28. The method of claim 18, wherein the activity of said RBM6-CSF1R fusion polypeptide and/or said truncated active CSF1R polypeptide is detected.

29. A method for determining whether a compound inhibits the progression of a cancer characterized by a RBM6-CSF1R fusion polynucleotide, a truncated CSF1R polynucleotide, a RBM6-CSF1R fusion polypeptide, and/or a truncated active CSF1R kinase polypeptide, said method comprising the step of determining whether said compound inhibits the expression and/or activity of said RBM6-CSF1R fusion polypeptide or said truncated active CSF1R kinase polypeptide in said cancer.

30. The method of claim 29, wherein inhibition of expression and/or activity of said RBM6-CSF1R fusion polypeptide or said truncated active CSF1R kinase polypeptide is determined using at least one reagent that detects a polynucleotide of claim 1 and/or at least one reagent of claim 15.

31. A method for inhibiting the progression of a cancer that expresses a RBM6-CSF1R fusion polypeptide and/or a truncated active CSF1R kinase polypeptide, said method comprising the step of inhibiting the expression and/or activity of said RBM6-CSF1R fusion polypeptide and/or said truncated active CSF1R kinase polypeptide in said cancer.

32. The method of claim 31, wherein said cancer is leukemia.

33. The method of claim 32, wherein said leukemia is acute myelogenous leukemia (AML).

34. The method of claim 31, wherein expression and/or activity of RBM6-CSF1R fusion polypeptide or truncated active CSF1R polypeptide is inhibited with a composition comprising Imatinib mesylate (STI-571) and/or its analogues.

35. The method of claim 34, wherein said Imatinib mesylate (STI-571) analogue is SU11248 or GW2580.


SEQUENCE LISTING (22 sequences)

SEQ ID NO: 1 - protein, 435 aa, Homo sapiens
MWGDSRPANRTGPFRGSQEERFAPGWNRDYPPPPLKKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 2 - DNA, 1308 bp, Homo sapiens
atgtggggggattctcgacctgctaacagaactggaccttttcgtgggagccaagaagaaaggtttgctcccgggtggaacagggattatcctcctcctccccttaagaagtgggagttcccccggaacaacctgcagtttggtaagaccctcggagctggagcctttgggaaggtggtggaggccacggcctttggtctgggcaaggaggatgctgtcctgaaggtggctgtgaagatgctgaagtccacggcccatgctgatgagaaggaggccctcatgtccgagctgaagatcatgagccacctgggccagcacgagaacatcgtcaaccttctgggagcctgtacccatggaggccctgtactggtcatcacggagtactgttgctatggcgacctgctcaactttctgcgaaggaaggctgaggccatgctgggacccagcctgagccccggccaggaccccgagggaggcgtcgactataagaacatccacctcgagaagaaatatgtccgcagggacagtggcttctccagccagggtgtggacacctatgtggagatgaggcctgtctccacttcttcaaatgactccttctctgagcaagacctggacaaggaggatggacggcccctggagctccgggacctgcttcacttctccagccaagtagcccagggcatggccttcctcgcttccaagaattgcatccaccgggacgtggcagcgcgtaacgtgctgttgaccaatggtcatgtggccaagattggggacttcgggctggctagggacatcatgaatgactccaactacattgtcaagggcaatgcccgcctgcctgtgaagtggatggccccagagagcatctttgactgtgtctacacggttcagagcgacgtctggtcctatggcatcctcctctgggagatcttctcacttgggctgaatccctaccctggcatcctggtgaacagcaagttctataaactggtgaaggatggataccaaatggcccagcctgcatttgccccaaagaatatatacagcatcatgcaggcctgctgggccttggagcccacccacagacccaccttccagcagatctgctccttccttcaggagcaggcccaagaggacaggagagagcgggactataccaatctgccgagcagcagcagaagcggtggcagcggcagcagcagcagtgagctggaggaggagagctctagtgagcacctgacctgctgcgagcaaggggatatcgcccagcccttgctgcagcccaacaactatcagttctgctga

SEQ ID NO: 3 - protein, 1123 aa, Homo sapiens
MWGDSRPANRTGPFRGSQEERFAPGWNRDYPPPPLKSHAQERHSGNFPGRDSLPFDFQGHSGPPFANVEEHSFSYGARDGPHGDYRGGEGPGHDFRGGDFSSSDFQSRDSSQLDFRGRDIHSGDFRDREGPPMDYRGGDGTSMDYRGREAPHMNYRDRDAHAVDFRGRDAPPSDFRGRGTYDLDFRGRDGSHADFRGRDLSDLDFRAREQSRSDFRNRDVSDLDFRDKDGTQVDFRGRGSGTTDLDFRDRDTPHSDFRGRHRSRTDQDFRGREMGSCMEFKDREMPPVDPNILDYIQPSTQDREHSGMNVNRREESTHDHTIERPAFGIQKGEFEHSETREGETQGVAFEHESPADFQNSQSPVQDQDKSQLSGREEQSSDAGLFKEEGGLDFLGRQDTDYRSMEYRDVDHRLPGSQMFGYGQSKSFPEGKTARDAQRDLQDQDYRTGPSEEKPSRLIRLSGVPEDATKEEILNAFRTPDGMPVKNLQLKEYNTGYDYGYVCVEFSLLEDAIGCMEANQGTLMIQDKEVTLEYVSSLDFWYCKRCKANIGGHRSSCSFCKNPREVTEAKQELITYPQPQKTSIPAPLEKQPNQPLRPADKEPEPRKREEGQESRLGHQKREAERYLPPSRREGPTFRRDRERESWSGETRQDGESKTIMLKRIYRSTPPEVIVEVLEPYVRLTTANVRIIKNRTGPMGHTYGFIDLDSHAEALRVVKILQNLDPPFSIDGKMVAVNLATGKRRNDSGDHSDHMHYYQGKKYFRDRRGGGRNSDWSSDTNRQGQQSSSDCYIYDSATGYYYDPLAGTYYDPNTQQEVYVPQDPGLPEEEEIKEKKPTSQGKSSSKKEMSKRDGKEKKDRGVTRFQENASEGKAPAEDVFKKPLPPTVKKEESPPPPKVVNPLIGLLGEYGGDSDYEEEEEEEQTPPPQPRTAQPQKREEQTKKENEEDKLTDWNKLACLLCRRQFPNKEVLIKHQQLSDLHKQNLEIHRKIKQSEQELAYLERREREGKFKGRGNDRREKLQSFDSPERKRIKYSRETDSDRKLVDKEDIDTSSKGGCVQQATGWRKGTGLGYGHPGLASSEEAEGRMRGPSVGASGRTSKRQSNETYRDAVRRVMFARYKELD

SEQ ID NO: 4 - DNA, 3372 bp, Homo sapiens
atgtggggggattctcgacctgctaacagaactggaccttttcgtgggagccaagaagaaaggtttgctcccgggtggaacagggattatcctcctcctccccttaagagtcatgctcaagagagacactctggcaactttcctggcagagattcacttccctttgatttccaggggcattcggggcctccttttgcaaatgtagaggagcattctttcagctatggagctagagacggaccgcatggtgactatcgaggaggggagggacctggacatgatttcagggggggagatttttcgtcttctgatttccagagcagagattcatcacagttggacttcaggggtagggacatacattctggggattttcgggatagagaaggaccacctatggactataggggtggagatggtacttctatggattatagaggtagggaggcacctcatatgaactacagagacagggatgctcacgctgttgacttcagaggtagggatgctcctccatctgacttcaggggccggggcacttatgatttagattttagaggccgggatggatcccatgcagattttaggggaagggatttatcagatttggattttagggccagagaacagtcccgttctgattttaggaatagagatgtatctgatttggactttagagacaaagacggaacacaagtagactttagaggccgaggttcaggtactactgatctagactttagggacagggatacgccacattcagatttcagaggtagacaccgatctaggactgatcaggattttaggggcagagagatgggatcttgtatggaatttaaagatagggagatgccccctgtggatccaaatattttggattacattcagccctctacacaagatagagaacattctggtatgaatgtgaacaggagagaagaatccacacatgaccatacgatagaaaggcctgcttttggcattcagaagggagaatttgagcattcagaaacaagagaaggagaaacacaaggtgtagcctttgaacatgagtctccagcagactttcagaacagccaaagtccagttcaagaccaagataagtcacagctttctggacgtgaagagcagagttcagatgctggtctgtttaaagaagaaggcggtctggactttcttgggcggcaagacaccgattacagaagcatggagtaccgtgatgtggatcataggctgccaggaagccagatgtttggctatggccagagcaagtcttttccagagggcaaaactgcccgagatgcccaacgggaccttcaggatcaagattataggaccggcccaagtgaggagaaacccagcaggcttattcgattaagtggggtacctgaagatgccacaaaagaagagattcttaatgcttttcggactcctgatggcatgcctgtaaagaacttgcagttgaaggagtataacacaggttacgactatggctatgtctgcgtggagttttcactcttggaagatgccatcggatgcatggaggccaaccagggaactctaatgatccaggacaaagaagttaccctggagtatgtatcaagcctggatttttggtactgcaaacgatgtaaggcaaacattggtgggcaccgatcttcctgttcattctgcaagaacccaagagaagtgacagaggccaagcaagaattaataacctaccctcagcctcagaaaacatccataccagcaccattggaaaaacagcccaaccagcccctaagaccagctgataaggaacctgaacccaggaagagggaagaaggccaagagtcacgcttaggacatcaaaagagagaagcagaaaggtatctgcctccttctcgaagggaagggccaactttccgaagagaccgagagagggagtcatggtctggagagacacgccaggatggagagagcaaaactatcatgctaaagcgtatctatcgttccacaccacctgaggtgatagtggaagtgctggagccctatgtccgccttactactgccaacgtccgtatcatcaagaacagaacaggccctatggggcatacctatggctttattgacctcgactcccatgcggaagctcttcgtgtggtgaagatcttacagaaccttgatccgccatttagcattgatgggaagatggtagctgtaaacctggccactggaaaacgaagaaatgattctggggaccattctgaccacatgcattactatcagggtaaaaaatatttccgagataggaggggaggtggcagaaattcagactggtcttcagatacaaatcgacaaggacaacagtcatcatctgactgctacatatatgattctgctactggctactattatgaccccttggcaggaacttattatgaccccaatacccagcaagaagtctatgtgccccaggatcctggattacctgaggaagaagagatcaaggaaaaaaaacccaccagtcaaggaaagtcaagtagcaagaaggaaatgtctaaaagagatggcaaggagaaaaaagacagaggagtgacgaggtttcaggaaaatgccagtgaagggaaggcccctgcagaagacgtctttaagaagcccctgcctcctactgtgaagaaggaagagagtccccctccacctaaagtggtaaacccactgatcggcctcttgggtgaatatggaggagacagtgactatgaggaggaagaagaggaggaacagacccctcccccacagccccgcacagcacagccccagaagcgagaggagcaaaccaagaaggagaatgaagaagacaaactcactgactggaataaactggcttgtctgctttgcagaaggcagtttcccaataaagaagttctgatcaaacaccagcagctgtcagacctgcacaagcaaaacctggaaatccaccggaagataaaacagtctgagcaggagctagcctatctggaaaggagagaacgagagggaaagtttaaaggaagaggaaatgatcgcagggaaaagctccagtcttttgactctccagaaaggaaacggattaagtactccagggaaactgacagtgatcgtaaacttgttgataaagaagatatcgacactagcagcaaaggaggctgtgtccaacaggctactggctggaggaaagggacaggcctgggatatggccatcctggattggcttcatcagaggaggctgaaggccggatgaggggccccagtgttggagcctcaggaagaaccagcaaaagacagtccaacgagacttatcgagatgctgttcgaagagtcatgtttgctcgatataaagaactcgattaa

SEQ ID NO: 5 - protein, 972 aa, Homo sapiens
MGPGVLLLLLVATAWHGQGIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 6 - DNA, 2919 bp, Homo sapiens
atgggcccaggagttctgctgctcctgctggtggccacagcttggcatggtcagggaatcccagtgatagagcccagtgtccctgagctggtcgtgaagccaggagcaacggtgaccttgcgatgtgtgggcaatggcagcgtggaatgggatggccccccatcacctcactggaccctgtactctgatggctccagcagcatcctcagcaccaacaacgctaccttccaaaacacggggacctatcgctgcactgagcctggagaccccctgggaggcagcgccgccatccacctctatgtcaaagaccctgcccggccctggaacgtgctagcacaggaggtggtcgtgttcgaggaccaggacgcactactgccctgtctgctcacagacccggtgctggaagcaggcgtctcgctggtgcgtgtgcgtggccggcccctcatgcgccacaccaactactccttctcgccctggcatggcttcaccatccacagggccaagttcattcagagccaggactatcaatgcagtgccctgatgggtggcaggaaggtgatgtccatcagcatccggctgaaagtgcagaaagtcatcccagggcccccagccttgacactggtgcctgcagagctggtgcggattcgaggggaggctgcccagatcgtgtgctcagccagcagcgttgatgttaactttgatgtcttcctccaacacaacaacaccaagctcgcaatccctcaacaatctgactttcataataaccgttaccaaaaagtcctgaccctcaacctcgatcaagtagatttccaacatgccggcaactactcctgcgtggccagcaacgtgcagggcaagcactccacctccatgttcttccgggtggtagagagtgcctacttgaacttgagctctgagcagaacctcatccaggaggtgaccgtgggggaggggctcaacctcaaagtcatggtggaggcctacccaggcctgcaaggttttaactggacctacctgggacccttttctgaccaccagcctgagcccaagcttgctaatgctaccaccaaggacacatacaggcacaccttcaccctctctctgccccgcctgaagccctctgaggctggccgctactccttcctggccagaaacccaggaggctggagagctctgacgtttgagctcacccttcgataccccccagaggtaagcgtcatatggacattcatcaacggctctggcacccttttgtgtgctgcctctgggtacccccagcccaacgtgacatggctgcagtgcagtggccacactgataggtgtgatgaggcccaagtgctgcaggtctgggatgacccataccctgaggtcctgagccaggagcccttccacaaggtgacggtgcagagcctgctgactgttgagaccttagagcacaaccaaacctacgagtgcagggcccacaacagcgtggggagtggctcctgggccttcatacccatctctgcaggagcccacacgcatcccccggatgagttcctcttcacaccagtggtggtcgcctgcatgtccatcatggccttgctgctgctgctgctcctgctgctattgtacaagtataagcagaagcccaagtaccaggtccgctggaagatcatcgagagctatgagggcaacagttatactttcatcgaccccacgcagctgccttacaacgagaagtgggagttcccccggaacaacctgcagtttggtaagaccctcggagctggagcctttgggaaggtggtggaggccacggcctttggtctgggcaaggaggatgctgtcctgaaggtggctgtgaagatgctgaagtccacggcccatgctgatgagaaggaggccctcatgtccgagctgaagatcatgagccacctgggccagcacgagaacatcgtcaaccttctgggagcctgtacccatggaggccctgtactggtcatcacggagtactgttgctatggcgacctgctcaactttctgcgaaggaaggctgaggccatgctgggacccagcctgagccccggccaggaccccgagggaggcgtcgactataagaacatccacctcgagaagaaatatgtccgcagggacagtggcttctccagccagggtgtggacacctatgtggagatgaggcctgtctccacttcttcaaatgactccttctctgagcaagacctggacaaggaggatggacggcccctggagctccgggacctgcttcacttctccagccaagtagcccagggcatggccttcctcgcttccaagaattgcatccaccgggacgtggcagcgcgtaacgtgctgttgaccaatggtcatgtggccaagattggggacttcgggctggctagggacatcatgaatgactccaactacattgtcaagggcaatgcccgcctgcctgtgaagtggatggccccagagagcatctttgactgtgtctacacggttcagagcgacgtctggtcctatggcatcctcctctgggagatcttctcacttgggctgaatccctaccctggcatcctggtgaacagcaagttctataaactggtgaaggatggataccaaatggcccagcctgcatttgccccaaagaatatatacagcatcatgcaggcctgctgggccttggagcccacccacagacccaccttccagcagatctgctccttccttcaggagcaggcccaagaggacaggagagagcgggactataccaatctgccgagcagcagcagaagcggtggcagcggcagcagcagcagtgagctggaggaggagagctctagtgagcacctgacctgctgcgagcaaggggatatcgcccagcccttgctgcagcccaacaactatcagttctgctga

SEQ ID NO: 7 - DNA, 48 bp, Homo sapiens
gattatcctcctcctccccttaagaagtgggagttcccccggaacaac

SEQ ID NO: 8 - protein, 16 aa, Homo sapiens
DYPPPPLKKWEFPRNN

SEQ ID NO: 9 - DNA, 24 bp, Artificial Sequence
  Description of Artificial Sequence Synthetic oligonucleotide
gacctgctaacagaactggacctt

SEQ ID NO: 10 - DNA, 24 bp, Artificial Sequence
  Description of Artificial Sequence Synthetic oligonucleotide
ctctgaagtcaacagcgtgagcat

SEQ ID NO: 11 - DNA, 24 bp, Artificial Sequence
  Description of Artificial Sequence Synthetic oligonucleotide
atcgagagctatgagggcaacagt

SEQ ID NO: 12 - DNA, 24 bp, Artificial Sequence
  Description of Artificial Sequence Synthetic oligonucleotide
ccttccttcgcagaaagttgagca

SEQ ID NO: 13 - DNA, 24 bp, Artificial Sequence
  Description of Artificial Sequence Synthetic oligonucleotide
aaagttgagcaggtcgccatagca

SEQ ID NO: 14 - DNA, 24 bp, Artificial Sequence
  Description of Artificial Sequence Synthetic oligonucleotide
agcaacagtactccgtgatgacca

SEQ ID NO: 15 - DNA, 42 bp, Artificial Sequence
  Description of Artificial Sequence Synthetic oligonucleotide
tgtaaaaggagggccagttcctcgtgttctgaaatgggagca

SEQ ID NO: 16 - DNA, 34 bp, Artificial Sequence
  Description of Artificial Sequence Synthetic oligonucleotide
aacagctatgaccatgcaaactgcaggttgttcc

SEQ ID NO: 17 - DNA, 40 bp, Artificial Sequence
  Description of Artificial Sequence Synthetic oligonucleotide
aacagctatgaccatgatcttcacagccaccttcaggaca

SEQ ID NO: 18 - DNA, 20 bp, Artificial Sequence
  Description of Artificial Sequence Synthetic oligonucleotide
tggaaatcccatcaccatct

SEQ ID NO: 19 - DNA, 20 bp, Artificial Sequence
  Description of Artificial Sequence Synthetic oligonucleotide
gtcttctgggtggcagtgat

SEQ ID NO: 20 - DNA, 31 bp, Artificial Sequence
  Description of Artificial Sequence Synthetic oligonucleotide
cggaattcgataaaaagagatgtggggggat

SEQ ID NO: 21 - DNA, 33 bp, Artificial Sequence
  Description of Artificial Sequence Synthetic oligonucleotide
cggaattcaccatgaagtgggagttcccccgga

SEQ ID NO: 22 - DNA, 29 bp, Artificial Sequence
  Description of Artificial Sequence Synthetic oligonucleotide
cggaattccgtcaactcctcagcagaact